SEQUENCE LISTING (331 sequences)

SEQ ID NO: 1 - DNA, 10 bp, Artificial Sequence
  Description of Artificial Sequence Synthetic oligonucleotide
aaaaaactgg

SEQ ID NO: 2 - DNA, 10 bp, Artificial Sequence
  Description of Artificial Sequence Synthetic oligonucleotide
aaaaaagcat

SEQ ID NO: 3 - DNA, 10 bp, Artificial Sequence
  Description of Artificial Sequence Synthetic oligonucleotide
aaaaaatatc

SEQ ID NO: 4 - DNA, 10 bp, Artificial Sequence
  Description of Artificial Sequence Synthetic oligonucleotide
aaaaacactc

SEQ ID NO: 5 - DNA, 10 bp, Artificial Sequence
  Description of Artificial Sequence Synthetic oligonucleotide
aaaaactttg

SEQ ID NO: 6 - DNA, 10 bp, Artificial Sequence
  Description of Artificial Sequence Synthetic oligonucleotide
aaaaagggtt

SEQ ID NO: 7 - DNA, 50 bp, Artificial Sequence
  Description of Artificial Sequence Synthetic oligonucleotide
agatcggaagagcgtcgtgtagggaaagctgagcaaatgttatcgaggtc

SEQ ID NO: 8 - DNA, 25 bp, Artificial Sequence
  Description of Artificial Sequence Synthetic probe
tatggaggaccaggccttggtaaga

SEQ ID NO: 9 - DNA, 25 bp, Artificial Sequence
  Description of Artificial Sequence Synthetic probe
gcgcgtgttaaatatatccctgccg

SEQ ID NO: 10 - DNA, 25 bp, Artificial Sequence
  Description of Artificial Sequence Synthetic probe
ggtgttgcgcaacctgtttctgttc

SEQ ID NO: 11 - DNA, 25 bp, Artificial Sequence
  Description of Artificial Sequence Synthetic probe
gcggctcgtcaaatctttgaccttc

SEQ ID NO: 12 - DNA, 25 bp, Artificial Sequence
  Description of Artificial Sequence Synthetic probe
cagcctggttacccagttcttactg

SEQ ID NO: 13 - DNA, 25 bp, Artificial Sequence
  Description of Artificial Sequence Synthetic probe
ggtgagaacgaagacaagaaccgtc

SEQ ID NO: 14 - DNA, 25 bp, Artificial Sequence
  Description of Artificial Sequence Synthetic probe
aattcatcgggtgaccctgtggaag

SEQ ID NO: 15 - DNA, 25 bp, Artificial Sequence
  Description of Artificial Sequence Synthetic probe
attgtggatcgtgttccagccttgg

SEQ ID NO: 16 - DNA, 25 bp, Artificial Sequence
  Description of Artificial Sequence Synthetic probe
aggtgtcaatgccatgcgtggtgaa

SEQ ID NO: 17 - DNA, 25 bp, Artificial Sequence
  Description of Artificial Sequence Synthetic probe
cacacctgatgtggtacacgtgatg

SEQ ID NO: 18 - DNA, 25 bp, Artificial Sequence
  Description of Artificial Sequence Synthetic probe
cgacgggatttatcggtggctttac

SEQ ID NO: 19 - DNA, 25 bp, Artificial Sequence
  Description of Artificial Sequence Synthetic probe
ttgtccaggtggcagaagatactcg

SEQ ID NO: 20 - DNA, 25 bp, Artificial Sequence
  Description of Artificial Sequence Synthetic probe
cttcagcgttgtctgtcgccagtaa

SEQ ID NO: 21 - DNA, 25 bp, Artificial Sequence
  Description of Artificial Sequence Synthetic probe
caacacgacgaatcagttcactggc

SEQ ID NO: 22 - DNA, 25 bp, Artificial Sequence
  Description of Artificial Sequence Synthetic probe
cctagtgagattgtccgtgacttgc

SEQ ID NO: 23 - DNA, 25 bp, Artificial Sequence
  Description of Artificial Sequence Synthetic probe
gaattagccaagtttgagcgtccgg

SEQ ID NO: 24 - DNA, 25 bp, Artificial Sequence
  Description of Artificial Sequence Synthetic probe
gcccaccttacccatagaaatggtc

SEQ ID NO: 25 - DNA, 25 bp, Artificial Sequence
  Description of Artificial Sequence Synthetic probe
caagtctaagacatcctgctccgtg

SEQ ID NO: 26 - DNA, 25 bp, Artificial Sequence
  Description of Artificial Sequence Synthetic probe
ggcccacatactcatcaaggttgac

SEQ ID NO: 27 - DNA, 25 bp, Artificial Sequence
  Description of Artificial Sequence Synthetic probe
attcaagtgggctacttcctgtcgc

SEQ ID NO: 28 - DNA, 25 bp, Artificial Sequence
  Description of Artificial Sequence Synthetic probe
catcctcgctagcaattgcagctag

SEQ ID NO: 29 - DNA, 25 bp, Artificial Sequence
  Description of Artificial Sequence Synthetic probe
tggcctgaggatagaaaccaatccc

SEQ ID NO: 30 - DNA, 25 bp, Artificial Sequence
  Description of Artificial Sequence Synthetic probe
gattcttctgtcgcagaagccaagc

SEQ ID NO: 31 - DNA, 25 bp, Artificial Sequence
  Description of Artificial Sequence Synthetic probe
ttactctcatccgcattagccgacg

SEQ ID NO: 32 - DNA, 25 bp, Artificial Sequence
  Description of Artificial Sequence Synthetic probe
aatgccacactacggtgttgtccac

SEQ ID NO: 33 - DNA, 25 bp, Artificial Sequence
  Description of Artificial Sequence Synthetic probe
cttggcagaatcggctcaatcaagg

SEQ ID NO: 34 - DNA, 25 bp, Artificial Sequence
  Description of Artificial Sequence Synthetic probe
gccgcaaagaagacaccagcatcta

SEQ ID NO: 35 - DNA, 25 bp, Artificial Sequence
  Description of Artificial Sequence Synthetic probe
accacagaaagggcggttaataggg

SEQ ID NO: 36 - DNA, 25 bp, Artificial Sequence
  Description of Artificial Sequence Synthetic probe
cgtgccctgttggaaaggcaattga

SEQ ID NO: 37 - DNA, 25 bp, Artificial Sequence
  Description of Artificial Sequence Synthetic probe
cgataccttgtcccatagctccact

SEQ ID NO: 38 - DNA, 25 bp, Artificial Sequence
  Description of Artificial Sequence Synthetic probe
ttgacctcagcgattacctgcaagc

SEQ ID NO: 39 - DNA, 25 bp, Artificial Sequence
  Description of Artificial Sequence Synthetic probe
ggctggatttgctccagcttcatct

SEQ ID NO: 40 - DNA, 25 bp, Artificial Sequence
  Description of Artificial Sequence Synthetic probe
agagcttctttcatgagtggagccc

SEQ ID NO: 41 - DNA, 25 bp, Artificial Sequence
  Description of Artificial Sequence Synthetic probe
taacgctccaattccgcatcagtcg

SEQ ID NO: 42 - DNA, 25 bp, Artificial Sequence
  Description of Artificial Sequence Synthetic probe
gccgcccttgagcctgatttgatta

SEQ ID NO: 43 - DNA, 25 bp, Artificial Sequence
  Description of Artificial Sequence Synthetic probe
ccaaccgttctcttccaagcaagca

SEQ ID NO: 44 - DNA, 25 bp, Artificial Sequence
  Description of Artificial Sequence Synthetic probe
cttggctcaagtcatgctccatctg

SEQ ID NO: 45 - DNA, 25 bp, Artificial Sequence
  Description of Artificial Sequence Synthetic probe
ctgtcacaacgggaacacgggtata

SEQ ID NO: 46 - DNA, 25 bp, Artificial Sequence
  Description of Artificial Sequence Synthetic probe
ccgcttcgagcaattgctcaaagac

SEQ ID NO: 47 - DNA, 25 bp, Artificial Sequence
  Description of Artificial Sequence Synthetic probe
ggtaagaaacagaacctgaagcgcc

SEQ ID NO: 48 - DNA, 25 bp, Artificial Sequence
  Description of Artificial Sequence Synthetic probe
atagctggacgcatgaggttgactg

SEQ ID NO: 49 - DNA, 25 bp, Artificial Sequence
  Description of Artificial Sequence Synthetic probe
actcttgtgactagagcaccgtgag

SEQ ID NO: 50 - DNA, 25 bp, Artificial Sequence
  Description of Artificial Sequence Synthetic probe
ggacgggtaaagcgtgagatttgtg

SEQ ID NO: 51 - DNA, 25 bp, Artificial Sequence
  Description of Artificial Sequence Synthetic probe
tcagccaaaccgttcaagactcctg

SEQ ID NO: 52 - DNA, 25 bp, Artificial Sequence
  Description of Artificial Sequence Synthetic probe
cgtggacgagtcagatagacacgat

SEQ ID NO: 53 - DNA, 25 bp, Artificial Sequence
  Description of Artificial Sequence Synthetic probe
acgttctaaccaagcttgacagccc

SEQ ID NO: 54 - DNA, 25 bp, Artificial Sequence
  Description of Artificial Sequence Synthetic probe
ctacttctgcagccagttctggatg

SEQ ID NO: 55 - DNA, 25 bp, Artificial Sequence
  Description of Artificial Sequence Synthetic probe
cgccacggtctgcaacatgttcttt

SEQ ID NO: 56 - DNA, 25 bp, Artificial Sequence
  Description of Artificial Sequence Synthetic probe
cacccgggtctctcatataagttgg

SEQ ID NO: 57 - DNA, 25 bp, Artificial Sequence
  Description of Artificial Sequence Synthetic probe
tcccacgaatcttagcacctgttgc

SEQ ID NO: 58 - DNA, 25 bp, Artificial Sequence
  Description of Artificial Sequence Synthetic probe
gctgcgcgctccatttcaaatagag

SEQ ID NO: 59 - DNA, 25 bp, Artificial Sequence
  Description of Artificial Sequence Synthetic probe
agaatggcacgttggagaacgatgg

SEQ ID NO: 60 - DNA, 25 bp, Artificial Sequence
  Description of Artificial Sequence Synthetic probe
cctgaagaaggtaagagtctcaccc

SEQ ID NO: 61 - DNA, 25 bp, Artificial Sequence
  Description of Artificial Sequence Synthetic probe
aaggcaagccaagtcagtatggctg

SEQ ID NO: 62 - DNA, 25 bp, Artificial Sequence
  Description of Artificial Sequence Synthetic probe
agtcaactgactggcatctacaccg

SEQ ID NO: 63 - DNA, 25 bp, Artificial Sequence
  Description of Artificial Sequence Synthetic probe
atttcggccaaagggagccacattg

SEQ ID NO: 64 - DNA, 25 bp, Artificial Sequence
  Description of Artificial Sequence Synthetic probe
gtgcggttcggagatacgcaagtaa

SEQ ID NO: 65 - DNA, 25 bp, Artificial Sequence
  Description of Artificial Sequence Synthetic probe
gacactattgaacgacgtgctgacg

SEQ ID NO: 66 - DNA, 25 bp, Artificial Sequence
  Description of Artificial Sequence Synthetic probe
catcgttggcgtattcgtcagtacc

SEQ ID NO: 67 - DNA, 25 bp, Artificial Sequence
  Description of Artificial Sequence Synthetic probe
ttccatggcaaccagcatagcatcc

SEQ ID NO: 68 - DNA, 25 bp, Artificial Sequence
  Description of Artificial Sequence Synthetic probe
ctggtgctgaggacaagtacaagga

SEQ ID NO: 69 - DNA, 25 bp, Artificial Sequence
  Description of Artificial Sequence Synthetic probe
tttctcaagtttcttcggcggaggc

SEQ ID NO: 70 - DNA, 25 bp, Artificial Sequence
  Description of Artificial Sequence Synthetic probe
gattggtccaatagtgcccgatacg

SEQ ID NO: 71 - DNA, 25 bp, Artificial Sequence
  Description of Artificial Sequence Synthetic probe
ttcctcttctgccagtctatgctgg

SEQ ID NO: 72 - DNA, 25 bp, Artificial Sequence
  Description of Artificial Sequence Synthetic probe
ccttgcagttggttcgaaaccaagg

SEQ ID NO: 73 - DNA, 25 bp, Artificial Sequence
  Description of Artificial Sequence Synthetic probe
ggcatacggttggatttcggttgca

SEQ ID NO: 74 - DNA, 25 bp, Artificial Sequence
  Description of Artificial Sequence Synthetic probe
gaggtccaaacgattctcaacctgc

SEQ ID NO: 75 - DNA, 25 bp, Artificial Sequence
  Description of Artificial Sequence Synthetic probe
gctgaacgaacattggccagacttg

SEQ ID NO: 76 - DNA, 25 bp, Artificial Sequence
  Description of Artificial Sequence Synthetic probe
cttggcctgctctctcgtttcaaac

SEQ ID NO: 77 - DNA, 25 bp, Artificial Sequence
  Description of Artificial Sequence Synthetic probe
aaaggcaatggactcttccaagccc

SEQ ID NO: 78 - DNA, 25 bp, Artificial Sequence
  Description of Artificial Sequence Synthetic probe
tatcggttgggtacgttcaggtgct

SEQ ID NO: 79 - DNA, 25 bp, Artificial Sequence
  Description of Artificial Sequence Synthetic probe
caattccctgtctcagctagatccg

SEQ ID NO: 80 - DNA, 25 bp, Artificial Sequence
  Description of Artificial Sequence Synthetic probe
ctcctgaatagcagacagataggcg

SEQ ID NO: 81 - DNA, 25 bp, Artificial Sequence
  Description of Artificial Sequence Synthetic probe
aagaccagagccgaaattccgtgtg

SEQ ID NO: 82 - DNA, 25 bp, Artificial Sequence
  Description of Artificial Sequence Synthetic probe
catccatgagacgagtcatggtgtc

SEQ ID NO: 83 - DNA, 25 bp, Artificial Sequence
  Description of Artificial Sequence Synthetic probe
agtttgacggttctcaggtacacgg

SEQ ID NO: 84 - DNA, 25 bp, Artificial Sequence
  Description of Artificial Sequence Synthetic probe
tgaagggcttgattagccgtgaacg

SEQ ID NO: 85 - DNA, 25 bp, Artificial Sequence
  Description of Artificial Sequence Synthetic probe
tccactctggtggtttatccgcatc

SEQ ID NO: 86 - DNA, 25 bp, Artificial Sequence
  Description of Artificial Sequence Synthetic probe
ctgccatgccactagtagcaccaaa

SEQ ID NO: 87 - DNA, 25 bp, Artificial Sequence
  Description of Artificial Sequence Synthetic probe
gccatctccacgatcattgaggcta

SEQ ID NO: 88 - DNA, 25 bp, Artificial Sequence
  Description of Artificial Sequence Synthetic probe
agtcgctcaaactgttaacgccacc

SEQ ID NO: 89 - DNA, 25 bp, Artificial Sequence
  Description of Artificial Sequence Synthetic probe
aaacggtgatggagtggtccagcat

SEQ ID NO: 90 - DNA, 25 bp, Artificial Sequence
  Description of Artificial Sequence Synthetic probe
gtgcccactctatcgcttcttctag

SEQ ID NO: 91 - DNA, 25 bp, Artificial Sequence
  Description of Artificial Sequence Synthetic probe
gtccgaactagcttgcttgttgagg

SEQ ID NO: 92 - DNA, 25 bp, Artificial Sequence
  Description of Artificial Sequence Synthetic probe
tcgtactgggcaggtgtcatgatgt

SEQ ID NO: 93 - DNA, 25 bp, Artificial Sequence
  Description of Artificial Sequence Synthetic probe
caaaggaagcctgtaagcgtgtctg

SEQ ID NO: 94 - DNA, 25 bp, Artificial Sequence
  Description of Artificial Sequence Synthetic probe
accaaaccttcaagaagcggagcca

SEQ ID NO: 95 - DNA, 25 bp, Artificial Sequence
  Description of Artificial Sequence Synthetic probe
tagcagtcataggtgcctcctggtt

SEQ ID NO: 96 - DNA, 25 bp, Artificial Sequence
  Description of Artificial Sequence Synthetic probe
ttccagcgagctgcgtcaaattgac

SEQ ID NO: 97 - DNA, 25 bp, Artificial Sequence
  Description of Artificial Sequence Synthetic probe
tgatggcttggatgactttgcgagc

SEQ ID NO: 98 - DNA, 25 bp, Artificial Sequence
  Description of Artificial Sequence Synthetic probe
ccaccagataattgacgggcaaagc

SEQ ID NO: 99 - DNA, 25 bp, Artificial Sequence
  Description of Artificial Sequence Synthetic probe
gttgaggcaacgaaggagggtactt

SEQ ID NO: 100 - DNA, 25 bp, Artificial Sequence
  Description of Artificial Sequence Synthetic probe
caacctgacgtccacctgcataaga

SEQ ID NO: 101 - DNA, 25 bp, Artificial Sequence
  Description of Artificial Sequence Synthetic probe
ccgtgagtacgaattcctccatcag

SEQ ID NO: 102 - DNA, 25 bp, Artificial Sequence
  Description of Artificial Sequence Synthetic probe
gtatcctctatcgtttggcggagga

SEQ ID NO: 103 - DNA, 25 bp, Artificial Sequence
  Description of Artificial Sequence Synthetic probe
gttcacttgcgactggtcaaacacc

SEQ ID NO: 104 - DNA, 25 bp, Artificial Sequence
  Description of Artificial Sequence Synthetic probe
tagaccgcgactgagttcgtttgca

SEQ ID NO: 105 - DNA, 25 bp, Artificial Sequence
  Description of Artificial Sequence Synthetic probe
ctatccacaccaccacgcttatgga

SEQ ID NO: 106 - DNA, 25 bp, Artificial Sequence
  Description of Artificial Sequence Synthetic probe
gttcttgcggttcatctgttccacc

SEQ ID NO: 107 - DNA, 25 bp, Artificial Sequence
  Description of Artificial Sequence Synthetic probe
aagtaaccacctgctgagagcaagg

SEQ ID NO: 108 - DNA, 25 bp, Artificial Sequence
  Description of Artificial Sequence Synthetic probe
ggagcaggtgctgacacttcttcat

SEQ ID NO: 109 - DNA, 25 bp, Artificial Sequence
  Description of Artificial Sequence Synthetic probe
cacctccgcatagctctttccttct

SEQ ID NO: 110 - DNA, 25 bp, Artificial Sequence
  Description of Artificial Sequence Synthetic probe
cgtccctcttaaagaagcaagccgt

SEQ ID NO: 111 - DNA, 25 bp, Artificial Sequence
  Description of Artificial Sequence Synthetic probe
gatttcaccaccaaacttcctcggg

SEQ ID NO: 112 - DNA, 25 bp, Artificial Sequence
  Description of Artificial Sequence Synthetic probe
tcagctgcatttggatctgctccac

SEQ ID NO: 113 - DNA, 25 bp, Artificial Sequence
  Description of Artificial Sequence Synthetic probe
tcattcacaccttcatctggccgag

SEQ ID NO: 114 - DNA, 25 bp, Artificial Sequence
  Description of Artificial Sequence Synthetic probe
ctgtatcgagtcacatggtccagca

SEQ ID NO: 115 - DNA, 25 bp, Artificial Sequence
  Description of Artificial Sequence Synthetic probe
aaggacgagcatatcctctatgccc

SEQ ID NO: 116 - DNA, 25 bp, Artificial Sequence
  Description of Artificial Sequence Synthetic probe
ccattaggattccaggtcccattgc

SEQ ID NO: 117 - DNA, 25 bp, Artificial Sequence
  Description of Artificial Sequence Synthetic probe
cgcaaactcgataatgagctggagg

SEQ ID NO: 118 - DNA, 25 bp, Artificial Sequence
  Description of Artificial Sequence Synthetic probe
gagtacactccagatgtaacggctc

SEQ ID NO: 119 - DNA, 25 bp, Artificial Sequence
  Description of Artificial Sequence Synthetic probe
tcggtggtggagattcaagctcaag

SEQ ID NO: 120 - DNA, 25 bp, Artificial Sequence
  Description of Artificial Sequence Synthetic probe
acctgcaggttgatgaacgagatcg

SEQ ID NO: 121 - DNA, 25 bp, Artificial Sequence
  Description of Artificial Sequence Synthetic probe
caatctcttggtcttggacgagcca

SEQ ID NO: 122 - DNA, 25 bp, Artificial Sequence
  Description of Artificial Sequence Synthetic probe
cacggagactcttgacactagactc

SEQ ID NO: 123 - DNA, 25 bp, Artificial Sequence
  Description of Artificial Sequence Synthetic probe
agggcaccaagaaaggcttcaaagg

SEQ ID NO: 124 - DNA, 100 bp, Artificial Sequence
  Description of Artificial Sequence Synthetic probe
gcgcgtgttaaatatatccctgccgagatcggaagagcgtcgtgtagggaaagctgagcaaatgttatcgaggtctatggaggaccaggccttggtaaga

SEQ ID NO: 125 - DNA, 100 bp, Artificial Sequence
  Description of Artificial Sequence Synthetic probe
gcggctcgtcaaatctttgaccttcagatcggaagagcgtcgtgtagggaaagctgagcaaatgttatcgaggtcggtgttgcgcaacctgtttctgttc

SEQ ID NO: 126 - DNA, 100 bp, Artificial Sequence
  Description of Artificial Sequence Synthetic probe
ggtgagaacgaagacaagaaccgtcagatcggaagagcgtcgtgtagggaaagctgagcaaatgttatcgaggtccagcctggttacccagttcttactg

SEQ ID NO: 127 - DNA, 100 bp, Artificial Sequence
  Description of Artificial Sequence Synthetic probe
attgtggatcgtgttccagccttggagatcggaagagcgtcgtgtagggaaagctgagcaaatgttatcgaggtcaattcatcgggtgaccctgtggaag

SEQ ID NO: 128 - DNA, 100 bp, Artificial Sequence
  Description of Artificial Sequence Synthetic probe
cacacctgatgtggtacacgtgatgagatcggaagagcgtcgtgtagggaaagctgagcaaatgttatcgaggtcaggtgtcaatgccatgcgtggtgaa

SEQ ID NO: 129 - DNA, 100 bp, Artificial Sequence
  Description of Artificial Sequence Synthetic probe
ttgtccaggtggcagaagatactcgagatcggaagagcgtcgtgtagggaaagctgagcaaatgttatcgaggtccgacgggatttatcggtggctttac

SEQ ID NO: 130 - DNA, 100 bp, Artificial Sequence
  Description of Artificial Sequence Synthetic probe
caacacgacgaatcagttcactggcagatcggaagagcgtcgtgtagggaaagctgagcaaatgttatcgaggtccttcagcgttgtctgtcgccagtaa

SEQ ID NO: 131 - DNA, 100 bp, Artificial Sequence
  Description of Artificial Sequence Synthetic probe
gaattagccaagtttgagcgtccggagatcggaagagcgtcgtgtagggaaagctgagcaaatgttatcgaggtccctagtgagattgtccgtgacttgc

SEQ ID NO: 132 - DNA, 100 bp, Artificial Sequence
  Description of Artificial Sequence Synthetic probe
caagtctaagacatcctgctccgtgagatcggaagagcgtcgtgtagggaaagctgagcaaatgttatcgaggtcgcccaccttacccatagaaatggtc

SEQ ID NO: 133 - DNA, 100 bp, Artificial Sequence
  Description of Artificial Sequence Synthetic probe
attcaagtgggctacttcctgtcgcagatcggaagagcgtcgtgtagggaaagctgagcaaatgttatcgaggtcggcccacatactcatcaaggttgac

SEQ ID NO: 134 - DNA, 100 bp, Artificial Sequence
  Description of Artificial Sequence Synthetic probe
tggcctgaggatagaaaccaatcccagatcggaagagcgtcgtgtagggaaagctgagcaaatgttatcgaggtccatcctcgctagcaattgcagctag

SEQ ID NO: 135 - DNA, 100 bp, Artificial Sequence
  Description of Artificial Sequence Synthetic probe
ttactctcatccgcattagccgacgagatcggaagagcgtcgtgtagggaaagctgagcaaatgttatcgaggtcgattcttctgtcgcagaagccaagc

SEQ ID NO: 136 - DNA, 100 bp, Artificial Sequence
  Description of Artificial Sequence Synthetic probe
cttggcagaatcggctcaatcaaggagatcggaagagcgtcgtgtagggaaagctgagcaaatgttatcgaggtcaatgccacactacggtgttgtccac

SEQ ID NO: 137 - DNA, 100 bp, Artificial Sequence
  Description of Artificial Sequence Synthetic probe
accacagaaagggcggttaatagggagatcggaagagcgtcgtgtagggaaagctgagcaaatgttatcgaggtcgccgcaaagaagacaccagcatcta

SEQ ID NO: 138 - DNA, 100 bp, Artificial Sequence
  Description of Artificial Sequence Synthetic probe
cgataccttgtcccatagctccactagatcggaagagcgtcgtgtagggaaagctgagcaaatgttatcgaggtccgtgccctgttggaaaggcaattga

SEQ ID NO: 139 - DNA, 100 bp, Artificial Sequence
  Description of Artificial Sequence Synthetic probe
ggctggatttgctccagcttcatctagatcggaagagcgtcgtgtagggaaagctgagcaaatgttatcgaggtcttgacctcagcgattacctgcaagc

SEQ ID NO: 140 - DNA, 100 bp, Artificial Sequence
  Description of Artificial Sequence Synthetic probe
taacgctccaattccgcatcagtcgagatcggaagagcgtcgtgtagggaaagctgagcaaatgttatcgaggtcagagcttctttcatgagtggagccc

SEQ ID NO: 141 - DNA, 100 bp, Artificial Sequence
  Description of Artificial Sequence Synthetic probe
ccaaccgttctcttccaagcaagcaagatcggaagagcgtcgtgtagggaaagctgagcaaatgttatcgaggtcgccgcccttgagcctgatttgatta

SEQ ID NO: 142 - DNA, 100 bp, Artificial Sequence
  Description of Artificial Sequence Synthetic probe
ctgtcacaacgggaacacgggtataagatcggaagagcgtcgtgtagggaaagctgagcaaatgttatcgaggtccttggctcaagtcatgctccatctg

SEQ ID NO: 143 - DNA, 100 bp, Artificial Sequence
  Description of Artificial Sequence Synthetic probe
ggtaagaaacagaacctgaagcgccagatcggaagagcgtcgtgtagggaaagctgagcaaatgttatcgaggtcccgcttcgagcaattgctcaaagac

SEQ ID NO: 144 - DNA, 100 bp, Artificial Sequence
  Description of Artificial Sequence Synthetic probe
actcttgtgactagagcaccgtgagagatcggaagagcgtcgtgtagggaaagctgagcaaatgttatcgaggtcatagctggacgcatgaggttgactg

SEQ ID NO: 145 - DNA, 100 bp, Artificial Sequence
  Description of Artificial Sequence Synthetic probe
tcagccaaaccgttcaagactcctgagatcggaagagcgtcgtgtagggaaagctgagcaaatgttatcgaggtcggacgggtaaagcgtgagatttgtg

SEQ ID NO: 146 - DNA, 100 bp, Artificial Sequence
  Description of Artificial Sequence Synthetic probe
acgttctaaccaagcttgacagcccagatcggaagagcgtcgtgtagggaaagctgagcaaatgttatcgaggtccgtggacgagtcagatagacacgat

SEQ ID NO: 147 - DNA, 100 bp, Artificial Sequence
  Description of Artificial Sequence Synthetic probe
cgccacggtctgcaacatgttctttagatcggaagagcgtcgtgtagggaaagctgagcaaatgttatcgaggtcctacttctgcagccagttctggatg

SEQ ID NO: 148 - DNA, 100 bp, Artificial Sequence
  Description of Artificial Sequence Synthetic probe
tcccacgaatcttagcacctgttgcagatcggaagagcgtcgtgtagggaaagctgagcaaatgttatcgaggtccacccgggtctctcatataagttgg

SEQ ID NO: 149 - DNA, 100 bp, Artificial Sequence
  Description of Artificial Sequence Synthetic probe
agaatggcacgttggagaacgatggagatcggaagagcgtcgtgtagggaaagctgagcaaatgttatcgaggtcgctgcgcgctccatttcaaatagag

SEQ ID NO: 150 - DNA, 100 bp, Artificial Sequence
  Description of Artificial Sequence Synthetic probe
aaggcaagccaagtcagtatggctgagatcggaagagcgtcgtgtagggaaagctgagcaaatgttatcgaggtccctgaagaaggtaagagtctcaccc

SEQ ID NO: 151 - DNA, 100 bp, Artificial Sequence
  Description of Artificial Sequence Synthetic probe
atttcggccaaagggagccacattgagatcggaagagcgtcgtgtagggaaagctgagcaaatgttatcgaggtcagtcaactgactggcatctacaccg

SEQ ID NO: 152 - DNA, 100 bp, Artificial Sequence
  Description of Artificial Sequence Synthetic probe
gacactattgaacgacgtgctgacgagatcggaagagcgtcgtgtagggaaagctgagcaaatgttatcgaggtcgtgcggttcggagatacgcaagtaa

SEQ ID NO: 153 - DNA, 100 bp, Artificial Sequence
  Description of Artificial Sequence Synthetic probe
ttccatggcaaccagcatagcatccagatcggaagagcgtcgtgtagggaaagctgagcaaatgttatcgaggtccatcgttggcgtattcgtcagtacc

SEQ ID NO: 154 - DNA, 100 bp, Artificial Sequence
  Description of Artificial Sequence Synthetic probe
tttctcaagtttcttcggcggaggcagatcggaagagcgtcgtgtagggaaagctgagcaaatgttatcgaggtcctggtgctgaggacaagtacaagga

SEQ ID NO: 155 - DNA, 100 bp, Artificial Sequence
  Description of Artificial Sequence Synthetic probe
ttcctcttctgccagtctatgctggagatcggaagagcgtcgtgtagggaaagctgagcaaatgttatcgaggtcgattggtccaatagtgcccgatacg

SEQ ID NO: 156 - DNA, 100 bp, Artificial Sequence
  Description of Artificial Sequence Synthetic probe
ggcatacggttggatttcggttgcaagatcggaagagcgtcgtgtagggaaagctgagcaaatgttatcgaggtcccttgcagttggttcgaaaccaagg

SEQ ID NO: 157 - DNA, 100 bp, Artificial Sequence
  Description of Artificial Sequence Synthetic probe
gctgaacgaacattggccagacttgagatcggaagagcgtcgtgtagggaaagctgagcaaatgttatcgaggtcgaggtccaaacgattctcaacctgc

SEQ ID NO: 158 - DNA, 100 bp, Artificial Sequence
  Description of Artificial Sequence Synthetic probe
aaaggcaatggactcttccaagcccagatcggaagagcgtcgtgtagggaaagctgagcaaatgttatcgaggtccttggcctgctctctcgtttcaaac

SEQ ID NO: 159 - DNA, 100 bp, Artificial Sequence
  Description of Artificial Sequence Synthetic probe
caattccctgtctcagctagatccgagatcggaagagcgtcgtgtagggaaagctgagcaaatgttatcgaggtctatcggttgggtacgttcaggtgct

SEQ ID NO: 160 - DNA, 100 bp, Artificial Sequence
  Description of Artificial Sequence Synthetic probe
aagaccagagccgaaattccgtgtgagatcggaagagcgtcgtgtagggaaagctgagcaaatgttatcgaggtcctcctgaatagcagacagataggcg

SEQ ID NO: 161 - DNA, 100 bp, Artificial Sequence
  Description of Artificial Sequence Synthetic probe
agtttgacggttctcaggtacacggagatcggaagagcgtcgtgtagggaaagctgagcaaatgttatcgaggtccatccatgagacgagtcatggtgtc

SEQ ID NO: 162 - DNA, 100 bp, Artificial Sequence
  Description of Artificial Sequence Synthetic probe
tccactctggtggtttatccgcatcagatcggaagagcgtcgtgtagggaaagctgagcaaatgttatcgaggtctgaagggcttgattagccgtgaacg

SEQ ID NO: 163 - DNA, 100 bp, Artificial Sequence
  Description of Artificial Sequence Synthetic probe
gccatctccacgatcattgaggctaagatcggaagagcgtcgtgtagggaaagctgagcaaatgttatcgaggtcctgccatgccactagtagcaccaaa

SEQ ID NO: 164 - DNA, 100 bp, Artificial Sequence
  Description of Artificial Sequence Synthetic probe
aaacggtgatggagtggtccagcatagatcggaagagcgtcgtgtagggaaagctgagcaaatgttatcgaggtcagtcgctcaaactgttaacgccacc

SEQ ID NO: 165 - DNA, 100 bp, Artificial Sequence
  Description of Artificial Sequence Synthetic probe
gtccgaactagcttgcttgttgaggagatcggaagagcgtcgtgtagggaaagctgagcaaatgttatcgaggtcgtgcccactctatcgcttcttctag

SEQ ID NO: 166 - DNA, 100 bp, Artificial Sequence
  Description of Artificial Sequence Synthetic probe
caaaggaagcctgtaagcgtgtctgagatcggaagagcgtcgtgtagggaaagctgagcaaatgttatcgaggtctcgtactgggcaggtgtcatgatgt

SEQ ID NO: 167 - DNA, 100 bp, Artificial Sequence
  Description of Artificial Sequence Synthetic probe
tagcagtcataggtgcctcctggttagatcggaagagcgtcgtgtagggaaagctgagcaaatgttatcgaggtcaccaaaccttcaagaagcggagcca

SEQ ID NO: 168 - DNA, 100 bp, Artificial Sequence
  Description of Artificial Sequence Synthetic probe
tgatggcttggatgactttgcgagcagatcggaagagcgtcgtgtagggaaagctgagcaaatgttatcgaggtcttccagcgagctgcgtcaaattgac

SEQ ID NO: 169 - DNA, 100 bp, Artificial Sequence
  Description of Artificial Sequence Synthetic probe
gttgaggcaacgaaggagggtacttagatcggaagagcgtcgtgtagggaaagctgagcaaatgttatcgaggtcccaccagataattgacgggcaaagc

SEQ ID NO: 170 - DNA, 100 bp, Artificial Sequence
  Description of Artificial Sequence Synthetic probe
ccgtgagtacgaattcctccatcagagatcggaagagcgtcgtgtagggaaagctgagcaaatgttatcgaggtccaacctgacgtccacctgcataaga

SEQ ID NO: 171 - DNA, 100 bp, Artificial Sequence
  Description of Artificial Sequence Synthetic probe
gttcacttgcgactggtcaaacaccagatcggaagagcgtcgtgtagggaaagctgagcaaatgttatcgaggtcgtatcctctatcgtttggcggagga

SEQ ID NO: 172 - DNA, 100 bp, Artificial Sequence
  Description of Artificial Sequence Synthetic probe
ctatccacaccaccacgcttatggaagatcggaagagcgtcgtgtagggaaagctgagcaaatgttatcgaggtctagaccgcgactgagttcgtttgca

SEQ ID NO: 173 - DNA, 100 bp, Artificial Sequence
  Description of Artificial Sequence Synthetic probe
aagtaaccacctgctgagagcaaggagatcggaagagcgtcgtgtagggaaagctgagcaaatgttatcgaggtcgttcttgcggttcatctgttccacc

SEQ ID NO: 174 - DNA, 100 bp, Artificial Sequence
  Description of Artificial Sequence Synthetic probe
cacctccgcatagctctttccttctagatcggaagagcgtcgtgtagggaaagctgagcaaatgttatcgaggtcggagcaggtgctgacacttcttcat

SEQ ID NO: 175 - DNA, 100 bp, Artificial Sequence
  Description of Artificial Sequence Synthetic probe
gatttcaccaccaaacttcctcgggagatcggaagagcgtcgtgtagggaaagctgagcaaatgttatcgaggtccgtccctcttaaagaagcaagccgt

SEQ ID NO: 176 - DNA, 100 bp, Artificial Sequence
  Description of Artificial Sequence Synthetic probe
tcattcacaccttcatctggccgagagatcggaagagcgtcgtgtagggaaagctgagcaaatgttatcgaggtctcagctgcatttggatctgctccac

SEQ ID NO: 177 - DNA, 100 bp, Artificial Sequence
  Description of Artificial Sequence Synthetic probe
aaggacgagcatatcctctatgcccagatcggaagagcgtcgtgtagggaaagctgagcaaatgttatcgaggtcctgtatcgagtcacatggtccagca

SEQ ID NO: 178 - DNA, 100 bp, Artificial Sequence
  Description of Artificial Sequence Synthetic probe
cgcaaactcgataatgagctggaggagatcggaagagcgtcgtgtagggaaagctgagcaaatgttatcgaggtcccattaggattccaggtcccattgc

SEQ ID NO: 179 - DNA, 100 bp, Artificial Sequence
  Description of Artificial Sequence Synthetic probe
tcggtggtggagattcaagctcaagagatcggaagagcgtcgtgtagggaaagctgagcaaatgttatcgaggtcgagtacactccagatgtaacggctc

SEQ ID NO: 180 - DNA, 100 bp, Artificial Sequence
  Description of Artificial Sequence Synthetic probe
caatctcttggtcttggacgagccaagatcggaagagcgtcgtgtagggaaagctgagcaaatgttatcgaggtcacctgcaggttgatgaacgagatcg

SEQ ID NO: 181 - DNA, 100 bp, Artificial Sequence
  Description of Artificial Sequence Synthetic probe
agggcaccaagaaaggcttcaaaggagatcggaagagcgtcgtgtagggaaagctgagcaaatgttatcgaggtccacggagactcttgacactagactc

SEQ ID NO: 182 - DNA, 25 bp, Artificial Sequence
  Description of Artificial Sequence Synthetic probe
gcggtaatagggtgaacgttatggg

SEQ ID NO: 183 - DNA, 25 bp, Artificial Sequence
  Description of Artificial Sequence Synthetic probe
tacccaaccgttcacaggtggaaag

SEQ ID NO: 184 - DNA, 25 bp, Artificial Sequence
  Description of Artificial Sequence Synthetic probe
cggtaatagggtgaacgttatgggc

SEQ ID NO: 185 - DNA, 25 bp, Artificial Sequence
  Description of Artificial Sequence Synthetic probe
acccaaccgttcacaggtggaaagt

SEQ ID NO: 186 - DNA, 25 bp, Artificial Sequence
  Description of Artificial Sequence Synthetic probe
gttccagcgtttgcgttgatgcttc

SEQ ID NO: 187 - DNA, 25 bp, Artificial Sequence
  Description of Artificial Sequence Synthetic probe
tgaaatttccgcttcgcgggaccaa

SEQ ID NO: 188 - DNA, 25 bp, Artificial Sequence
  Description of Artificial Sequence Synthetic probe
ttccagcgtttgcgttgatgcttcg

SEQ ID NO: 189 - DNA, 25 bp, Artificial Sequence
  Description of Artificial Sequence Synthetic probe
gaaatttccgcttcgcgggaccaaa

SEQ ID NO: 190 - DNA, 25 bp, Artificial Sequence
  Description of Artificial Sequence Synthetic probe
tgatgccggtattcgctttggcgat

SEQ ID NO: 191 - DNA, 25 bp, Artificial Sequence
  Description of Artificial Sequence Synthetic probe
atgcgcgattatcccgatattcggc

SEQ ID NO: 192 - DNA, 25 bp, Artificial Sequence
  Description of Artificial Sequence Synthetic probe
tcgctttggcgatgcggtacaactt

SEQ ID NO: 193 - DNA, 25 bp, Artificial Sequence
  Description of Artificial Sequence Synthetic probe
cccgatattcggctggatatcgatg

SEQ ID NO: 194 - DNA, 25 bp, Artificial Sequence
  Description of Artificial Sequence Synthetic probe
gcctttgccatcgttttacgcgtga

SEQ ID NO: 195 - DNA, 25 bp, Artificial Sequence
  Description of Artificial Sequence Synthetic probe
gatcgtagcatcccctgcatacctt

SEQ ID NO: 196 - DNA, 25 bp, Artificial Sequence
  Description of Artificial Sequence Synthetic probe
cctttgccatcgttttacgcgtgag

SEQ ID NO: 197 - DNA, 25 bp, Artificial Sequence
  Description of Artificial Sequence Synthetic probe
atcgtagcatcccctgcataccttg

SEQ ID NO: 198 - DNA, 25 bp, Artificial Sequence
  Description of Artificial Sequence Synthetic probe
aattacgccggaagccgcgttaatg

SEQ ID NO: 199 - DNA, 25 bp, Artificial Sequence
  Description of Artificial Sequence Synthetic probe
tgcctttgccatcgttttacgcgtg

SEQ ID NO: 200 - DNA, 25 bp, Artificial Sequence
  Description of Artificial Sequence Synthetic probe
atgataaagcgttgcgtctctccgc

SEQ ID NO: 201 - DNA, 25 bp, Artificial Sequence
  Description of Artificial Sequence Synthetic probe
cgggctatatcggtgggagtttgtt

SEQ ID NO: 202 - DNA, 25 bp, Artificial Sequence
  Description of Artificial Sequence Synthetic probe
gaagacttacaggatgggcggttgt

SEQ ID NO: 203 - DNA, 25 bp, Artificial Sequence
  Description of Artificial Sequence Synthetic probe
atgataaagcgttgcgtctctccgc

SEQ ID NO: 204 - DNA, 25 bp, Artificial Sequence
  Description of Artificial Sequence Synthetic probe
cgaatggcaggactcgcttactgaa

SEQ ID NO: 205 - DNA, 25 bp, Artificial Sequence
  Description of Artificial Sequence Synthetic probe
acgggcaatgcacaaatcaaagcgg

SEQ ID NO: 206 - DNA, 25 bp, Artificial Sequence
  Description of Artificial Sequence Synthetic probe
tggcaggactcgcttactgaagatg

SEQ ID NO: 207 - DNA, 25 bp, Artificial Sequence
  Description of Artificial Sequence Synthetic probe
gcaatgcacaaatcaaagcggcggt

SEQ ID NO: 208 - DNA, 25 bp, Artificial Sequence
  Description of Artificial Sequence Synthetic probe
aaacttcggtgcaggggttaggcat

SEQ ID NO: 209 - DNA, 25 bp, Artificial Sequence
  Description of Artificial Sequence Synthetic probe
tggcagagcgagtgactatctgaag

SEQ ID NO: 210 - DNA, 25 bp, Artificial Sequence
  Description of Artificial Sequence Synthetic probe
ggctttggcaacgtaggcttcttca

SEQ ID NO: 211 - DNA, 25 bp, Artificial Sequence
  Description of Artificial Sequence Synthetic probe
atgcacatcacaccgtcctgaccaa

SEQ ID NO: 212 - DNA, 25 bp, Artificial Sequence
  Description of Artificial Sequence Synthetic probe
gcaggcgatctttaatcatctgcgg

SEQ ID NO: 213 - DNA, 25 bp, Artificial Sequence
  Description of Artificial Sequence Synthetic probe
aaacgctttcgcgttggcgaggtta

SEQ ID NO: 214 - DNA, 25 bp, Artificial Sequence
  Description of Artificial Sequence Synthetic probe
caggcgatctttaatcatctgcggg

SEQ ID NO: 215 - DNA, 25 bp, Artificial Sequence
  Description of Artificial Sequence Synthetic probe
aacgctttcgcgttggcgaggttaa

SEQ ID NO: 216 - DNA, 25 bp, Artificial Sequence
  Description of Artificial Sequence Synthetic probe
ctgcagatcgtcccagtcggattta

SEQ ID NO: 217 - DNA, 25 bp, Artificial Sequence
  Description of Artificial Sequence Synthetic probe
gtatgcagatcgtcaggatggccaa

SEQ ID NO: 218 - DNA, 25 bp, Artificial Sequence
  Description of Artificial Sequence Synthetic probe
atcagcgtctcacgtatcttttgat

SEQ ID NO: 219 - DNA, 25 bp, Artificial Sequence
  Description of Artificial Sequence Synthetic probe
gctcgttttgatccgatttctgttt

SEQ ID NO: 220 - DNA, 25 bp, Artificial Sequence
  Description of Artificial Sequence Synthetic probe
cgtctcacgtatcttttgatggaaa

SEQ ID NO: 221 - DNA, 25 bp, Artificial Sequence
  Description of Artificial Sequence Synthetic probe
ttttgatccgatttctgtttcgcca

SEQ ID NO: 222 - DNA, 25 bp, Artificial Sequence
  Description of Artificial Sequence Synthetic probe
gtctcacgtatcttttgatggaaac

SEQ ID NO: 223 - DNA, 25 bp, Artificial Sequence
  Description of Artificial Sequence Synthetic probe
tttgatccgatttctgtttcgccaa

SEQ ID NO: 224 - DNA, 25 bp, Artificial Sequence
  Description of Artificial Sequence Synthetic probe
tctcacgtatcttttgatggaaacg

SEQ ID NO: 225 - DNA, 25 bp, Artificial Sequence
  Description of Artificial Sequence Synthetic probe
ttgatccgatttctgtttcgccaat

SEQ ID NO: 226 - DNA, 25 bp, Artificial Sequence
  Description of Artificial Sequence Synthetic probe
gctcgttttgatccgatttctgttt

SEQ ID NO: 227 - DNA, 25 bp, Artificial Sequence
  Description of Artificial Sequence Synthetic probe
cgacgaatgcaatcaggtcaaaata

SEQ ID NO: 228 - DNA, 25 bp, Artificial Sequence
  Description of Artificial Sequence Synthetic probe
atcgacgaatgcaatcaggtcaaaa

SEQ ID NO: 229 - DNA, 25 bp, Artificial Sequence
  Description of Artificial Sequence Synthetic probe
acgcggtgtctccaatttagaataa

SEQ ID NO: 230 - DNA, 25 bp, Artificial Sequence
  Description of Artificial Sequence Synthetic probe
cgacgaatgcaatcaggtcaaaata

SEQ ID NO: 231 - DNA, 25 bp, Artificial Sequence
  Description of Artificial Sequence Synthetic probe
gcggtgtctccaatttagaataaca

SEQ ID NO: 232 - DNA, 25 bp, Artificial Sequence
  Description of Artificial Sequence Synthetic probe
aacgcggtgtctccaatttagaata

SEQ ID NO: 233 - DNA, 25 bp, Artificial Sequence
  Description of Artificial Sequence Synthetic probe
tctgcgacatattcaatatggtgct

SEQ ID NO: 234 - DNA, 25 bp, Artificial Sequence
  Description of Artificial Sequence Synthetic probe
acatattcaatatggtgctcgggaa

SEQ ID NO: 235 - DNA, 25 bp, Artificial Sequence
  Description of Artificial Sequence Synthetic probe
atcgtctcctgtgagataattgcat

SEQ ID NO: 236 - DNA, 25 bp, Artificial Sequence
  Description of Artificial Sequence Synthetic probe
ctgtgagataattgcatccgatcat

SEQ ID NO: 237 - DNA, 25 bp, Artificial Sequence
  Description of Artificial Sequence Synthetic probe
ccgtttctggttttgtcttgatgat

SEQ ID NO: 238 - DNA, 25 bp, Artificial Sequence
  Description of Artificial Sequence Synthetic probe
taattgcatccgatcatatagggct

SEQ ID NO: 239 - DNA, 25 bp, Artificial Sequence
  Description of Artificial Sequence Synthetic probe
ggttttgtcttgatgatcaaatccg

SEQ ID NO: 240 - DNA, 25 bp, Artificial Sequence
  Description of Artificial Sequence Synthetic probe
caaaacttgatatgaccgatctcac

SEQ ID NO: 241 - DNA, 25 bp, Artificial Sequence
  Description of Artificial Sequence Synthetic probe
gatatcgcgctatcggtaaactaat

SEQ ID NO: 242 - DNA, 25 bp, Artificial Sequence
  Description of Artificial Sequence Synthetic probe
catttacctctatcacttcggctaa

SEQ ID NO: 243 - DNA, 25 bp, Artificial Sequence
  Description of Artificial Sequence Synthetic probe
aatccgaacgaacacatagcatttg

SEQ ID NO: 244 - DNA, 25 bp, Artificial Sequence
  Description of Artificial Sequence Synthetic probe
tcatgtttgataaggcgacgaaaac

SEQ ID NO: 245 - DNA, 25 bp, Artificial Sequence
  Description of Artificial Sequence Synthetic probe
ggccttatctaaaccactgaagttt

SEQ ID NO: 246 - DNA, 25 bp, Artificial Sequence
  Description of Artificial Sequence Synthetic probe
catccttataggaacatcacagact

SEQ ID NO: 247 - DNA, 25 bp, Artificial Sequence
  Description of Artificial Sequence Synthetic probe
ggcatttccgtagcttttgaaattc

SEQ ID NO: 248 - DNA, 25 bp, Artificial Sequence
  Description of Artificial Sequence Synthetic probe
tccttataggaacatcacagacttc

SEQ ID NO: 249 - DNA, 25 bp, Artificial Sequence
  Description of Artificial Sequence Synthetic probe
catttccgtagcttttgaaattccc

SEQ ID NO: 250 - DNA, 25 bp, Artificial Sequence
  Description of Artificial Sequence Synthetic probe
ccttataggaacatcacagacttca

SEQ ID NO: 251 - DNA, 25 bp, Artificial Sequence
  Description of Artificial Sequence Synthetic probe
atttccgtagcttttgaaattcccc

SEQ ID NO: 252 - DNA, 25 bp, Artificial Sequence
  Description of Artificial Sequence Synthetic probe
cttataggaacatcacagacttcac

SEQ ID NO: 253 - DNA, 25 bp, Artificial Sequence
  Description of Artificial Sequence Synthetic probe
tttccgtagcttttgaaattcccct

SEQ ID NO: 254 - DNA, 25 bp, Artificial Sequence
  Description of Artificial Sequence Synthetic probe
ggtcgccgcgatcaaggagttcttc

SEQ ID NO: 255 - DNA, 25 bp, Artificial Sequence
  Description of Artificial Sequence Synthetic probe
catcgaaacgccgtaccgcaaggtg

SEQ ID NO: 256 - DNA, 25 bp, Artificial Sequence
  Description of Artificial Sequence Synthetic probe
gttcatcgaaacgccgtaccgcaag

SEQ ID NO: 257 - DNA, 25 bp, Artificial Sequence
  Description of Artificial Sequence Synthetic probe
acccaggacgtggaggcgatcacac

SEQ ID NO: 258 - DNA, 25 bp, Artificial Sequence
  Description of Artificial Sequence Synthetic probe
tcgaactcgacgtgcaaaacgagga

SEQ ID NO: 259 - DNA, 25 bp, Artificial Sequence
  Description of Artificial Sequence Synthetic probe
ggcgtattcgtatgcttcgatggcc

SEQ ID NO: 260 - DNA, 25 bp, Artificial Sequence
  Description of Artificial Sequence Synthetic probe
ctcgctccacaataagtttaagtgg

SEQ ID NO: 261 - DNA, 25 bp, Artificial Sequence
  Description of Artificial Sequence Synthetic probe
attcagctaccgcagaaaactctat

SEQ ID NO: 262 - DNA, 25 bp, Artificial Sequence
  Description of Artificial Sequence Synthetic probe
ctcgctccacaataagtttaagtgg

SEQ ID NO: 263 - DNA, 25 bp, Artificial Sequence
  Description of Artificial Sequence Synthetic probe
attcagctaccgcagaaaactctat

SEQ ID NO: 264 - DNA, 25 bp, Artificial Sequence
  Description of Artificial Sequence Synthetic probe
tgatggagtaaaagcccctgggata

SEQ ID NO: 265 - DNA, 25 bp, Artificial Sequence
  Description of Artificial Sequence Synthetic probe
ctttatgcctgatactgctatggct

SEQ ID NO: 266 - DNA, 25 bp, Artificial Sequence
  Description of Artificial Sequence Synthetic probe
tgatggagtaaaagcccctgggata

SEQ ID NO: 267 - DNA, 25 bp, Artificial Sequence
  Description of Artificial Sequence Synthetic probe
ctttatgcctgatactgctatggct

SEQ ID NO: 268 - DNA, 25 bp, Artificial Sequence
  Description of Artificial Sequence Synthetic probe
ataacagaggggatacctattgtat

SEQ ID NO: 269 - DNA, 25 bp, Artificial Sequence
  Description of Artificial Sequence Synthetic probe
cctcagttaaggttcaactttatgc

SEQ ID NO: 270 - DNA, 25 bp, Artificial Sequence
  Description of Artificial Sequence Synthetic probe
ataacagaggggatacctattgtat

SEQ ID NO: 271 - DNA, 25 bp, Artificial Sequence
  Description of Artificial Sequence Synthetic probe
cctcagttaaggttcaactttatgc

SEQ ID NO: 272 - DNA, 25 bp, Artificial Sequence
  Description of Artificial Sequence Synthetic probe
ataaatagtctatggagctttgatc

SEQ ID NO: 273 - DNA, 25 bp, Artificial Sequence
  Description of Artificial Sequence Synthetic probe
ttttatgccagaagctcgctccaca

SEQ ID NO: 274 - DNA, 25 bp, Artificial Sequence
  Description of Artificial Sequence Synthetic probe
ataaatagtctatggagctttgatc

SEQ ID NO: 275 - DNA, 25 bp, Artificial Sequence
  Description of Artificial Sequence Synthetic probe
ttttatgccagaagctcgctccaca

SEQ ID NO: 276 - DNA, 36 bp, Human papillomavirus
agttgttgcaacagcattgcgactatatctgggtta

SEQ ID NO: 277 - DNA, 36 bp, Human papillomavirus
agctgttgcaacagcattgtgactatatatgggtcc

SEQ ID NO: 278 - DNA, 36 bp, Human papillomavirus
agttattgcaacagcattgtgactatatttggatta

SEQ ID NO: 279 - DNA, 36 bp, Human papillomavirus
agctgttgcaacagcattgtgactatatctgggtcc

SEQ ID NO: 280 - DNA, 36 bp, Human papillomavirus
agctattgcaacagcattgtgactatatctgggtcc

SEQ ID NO: 281 - DNA, 36 bp, Human papillomavirus; identical to SEQ ID NO: 280

SEQ ID NO: 282 - DNA, 100 bp, Artificial Sequence
  Description of Artificial Sequence Synthetic polynucleotide
gtgaacatctcccggatctgatccgccgcttttaacaccagcgcgagccggaggatcgtggagggtgggagaaaaacgcaccggttcgcaccggggccgg

SEQ ID NO: 283 - DNA, 100 bp, Artificial Sequence
  Description of Artificial Sequence Synthetic polynucleotide
gtgaacatctcccggatctgatccgccgcttttcacaccagcgcgacccggcggatcgttgagggcgcgaggaaagcgaaccgggtaggaccggggccgg

SEQ ID NO: 284 - DNA, 100 bp, Artificial Sequence
  Description of Artificial Sequence Synthetic polynucleotide
gtgaacatctcccggatctgatccgccgcttttcccaccagcgcgaaccggcggagcgttgagggggcgaggcaagcgaaagggttagcaccggggccgg

SEQ ID NO: 285 - DNA, 100 bp, Artificial Sequence
  Description of Artificial Sequence Synthetic polynucleotide
gtgaacatctcccggatctgatccgccgcttttaccaccagggcgaccaggcggatcgtggagggtgcgagaaaagcgcaccggttaggaccggggccgg

SEQ ID NO: 286 - DNA, 100 bp, Artificial Sequence
  Description of Artificial Sequence Synthetic polynucleotide
gtgaacatctcccggatctgatacgccgcttttcccaccagcgcgatccggcggatcgttgagggagcgagcaaagcctaccggttagcaacggggccgg

SEQ ID NO: 287 - DNA, 100 bp, Artificial Sequence
  Description of Artificial Sequence Synthetic polynucleotide
gtgaacatctcccggatctgatccgccgcttttccaaccagcgcgaaccggcggatcgttgagggggcgagaaaagcagaccggttagcaccggagccgg

SEQ ID NO: 288 - DNA, 100 bp, Artificial Sequence
  Description of Artificial Sequence Synthetic polynucleotide
gtgaacatctcccggatctgatccgccgcttttcccaccagcgcgagccggcggatcgtggagggagggagcaaggcagcccggttagcaccggggccgg

SEQ ID NO: 289 - DNA, 100 bp, Artificial Sequence
  Description of Artificial Sequence Synthetic polynucleotide
gtgaacatctcccggatcagatccgccgcttttcacaacagcgcgagccgcaggaccgtggagggggcggcaaaaacagaacggttagcaccggggccgg

SEQ ID NO: 290 - DNA, 100 bp, Artificial Sequence
  Description of Artificial Sequence Synthetic polynucleotide
gtgaacatctcccggatctgatccgccgcttttcccaccagcgcgagccggcggatcgtggagggagggagaaaagccgcccggttagcaccggggccgg

SEQ ID NO: 291 - DNA, 100 bp, Artificial Sequence
  Description of Artificial Sequence Synthetic polynucleotide
gtgaacatctcccggatctgatccgccgcttttcccaccagcgcgaccaggcggcgcgtggagggggcgaggcaaccagaccggttagcaacggggccgg

SEQ ID NO: 292 - DNA, 100 bp, Artificial Sequence
  Description of Artificial Sequence Synthetic polynucleotide
gtgaacatctcccggatctgatccgccgcttttcacaccagcgagaaccggcggacagttgagggtgcgaggaaaggggaccgggtagcacgggtgccgg

SEQ ID NO: 293 - DNA, 100 bp, Artificial Sequence
  Description of Artificial Sequence Synthetic polynucleotide
gtgaacatctcccggatctgatccgccgcttttcccaccagcgagatccggcggatcgttgagggggcgagaacagcagacccgttagcaccggggccgg

SEQ ID NO: 294 - DNA, 100 bp, Artificial Sequence
  Description of Artificial Sequence Synthetic polynucleotide
gtgaacatctcccggatctgatccgccgcttttcccaccagcgcgagccggcggatcgttgagggggcgagcaaagcggaccgggtagaaccggggccgg

SEQ ID NO: 295 - DNA, 100 bp, Artificial Sequence
  Description of Artificial Sequence Synthetic polynucleotide
gtgaacatctcccggatctgatccgccgcttttcccaccagcgcgaaccggcggatcgtggagggcgcgaggaaaaccacccgggtagcaccggagccgg

SEQ ID NO: 296 - DNA, 75 bp, Unknown
  Description of Unknown Target genomic polynucleotide
gtgaacatctcccggatctgatccgccgctttacacaccagcgcgaggcggcggatcgtggagggagagagaaaa

SEQ ID NO: 297 - DNA, 75 bp, Artificial Sequence
  Description of Artificial Sequence Synthetic oligonucleotide
gtgaacatctcccggatctgatccgccgcttaccacaccagcgcgagccggcggatcgttgagggcgcgaggaca

SEQ ID NO: 298 - DNA, 75 bp, Artificial Sequence
  Description of Artificial Sequence Synthetic oligonucleotide
gtgaacatctcccggatctgatccgccgcttttaccaccagggcgaccaggcggatcgtggagggtgcgagaaaa

SEQ ID NO: 299 - DNA, 75 bp, Artificial Sequence
  Description of Artificial Sequence Synthetic oligonucleotide
gtgaacatctcccggatctgatccgccgcttttcccaccagcgcgatccggcggatcgttgagggagcgagcaaa

SEQ ID NO: 300 - DNA, 75 bp, Artificial Sequence
  Description of Artificial Sequence Synthetic oligonucleotide
gtgaacatctcccggatctgatccgccgcttttccaaccagcgcgaaccggcggatcgttgagggggcgagaaaa

SEQ ID NO: 301 - DNA, 75 bp, Artificial Sequence
  Description of Artificial Sequence Synthetic oligonucleotide
gtgaacatctcccggatctgatccgccgcttttcccaccagcgagatccggcggatcgttgagggggcgagaaca

SEQ ID NO: 302 - DNA, 75 bp, Artificial Sequence
  Description of Artificial Sequence Synthetic oligonucleotide
gtgaacatctcccggatctgatccgccgcttttcccaccagcgcgagccggcggatcgttgagggggcgagcaaa

SEQ ID NO: 303 - DNA, 75 bp, Artificial Sequence
  Description of Artificial Sequence Synthetic oligonucleotide
gtgaacatctcccggatctgatccgccgcttttcccaccagggcgagccggcggatcgttgagggggcgagcaaa

SEQ ID NO: 304 - DNA, 75 bp, Artificial Sequence
  Description of Artificial Sequence Synthetic oligonucleotide
gtgaacatctcccggatctgatccgccgcttttcccaccagcgcgagcagggggatcgttgaggggacgagcaaa

SEQ ID NO: 305 - DNA, 75 bp, Artificial Sequence
  Description of Artificial Sequence Synthetic oligonucleotide
gtgaacatctcccggatctgatccgccgcttttcccaccagcgcgagccggcggatcgtggagggagggagcaag

SEQ ID NO: 306 - DNA, 75 bp, Artificial Sequence
  Description of Artificial Sequence Synthetic oligonucleotide
gtgaacatctcccggatctgatccgccgcttttcccaccagcgcgagccggcggatcgtggagggagggagaaaa

SEQ ID NO: 307 - DNA, 75 bp, Artificial Sequence
  Description of Artificial Sequence Synthetic oligonucleotide
gtgaacatctcccggatctgatccgccgcttttcccaccagcgcgaaccggcggatcgtggagggcgcgaggaaa

SEQ ID NO: 308 - DNA, 75 bp, Artificial Sequence
  Description of Artificial Sequence Synthetic oligonucleotide
gtgaacatctcccggatctgatccgccgcttttcccagcagcgcgaaccggcggatcgttgagggtgggagaaaa

SEQ ID NO: 309 - DNA, 75 bp, Artificial Sequence
  Description of Artificial Sequence Synthetic oligonucleotide
gtgaacatctcccggatctgatccgccgattttcccaccagcgcgaccgggcggatcgttgagggagcgaggcaa

SEQ ID NO: 310 - DNA, 75 bp, Artificial Sequence
  Description of Artificial Sequence Synthetic oligonucleotide
gtgaacatctcccggatctgatccgccgcttttcccaccagcgcgaaccggaggatcgtggagggagcgggacaa

SEQ ID NO: 311 - DNA, 75 bp, Artificial Sequence
  Description of Artificial Sequence Synthetic oligonucleotide
gtgaacatctcccggatctgatccgccgcttttcccaccagcgcgaggcggcggctcgtggggggcgcgaggcaa

SEQ ID NO: 312 - DNA, 75 bp, Artificial Sequence
  Description of Artificial Sequence Synthetic oligonucleotide
gtgaacatctcccggatctgatccgccgcttttcccaccagcgcgaccaggcggcgcgtggagggggcgaggcaa

SEQ ID NO: 313 - DNA, 75 bp, Artificial Sequence
  Description of Artificial Sequence Synthetic oligonucleotide
gtgaacatctcccggatctgatccgccgctttacccaacagcgcgatccggcggaccggtgagggggcgagaaca

SEQ ID NO: 314 - DNA, 75 bp, Artificial Sequence
  Description of Artificial Sequence Synthetic oligonucleotide
gtgaacatctcccggatctgatccgccgcttttcccacgagcgcgggccggcgcatagttgggggtgggaggaaa

SEQ ID NO: 315 - DNA, 75 bp, Artificial Sequence
  Description of Artificial Sequence Synthetic oligonucleotide
gtgaacatctcccggatctgatccgccgctttacccaccagcgcgacgcggcggagcgtggggggcgcgaggaaa

SEQ ID NO: 316 - DNA, 75 bp, Artificial Sequence
  Description of Artificial Sequence Synthetic oligonucleotide
gtgaacatctcccggatctgatccgccgcttttcacaccagcgagaaccggcggacagttgagggtgcgaggaaa

SEQ ID NO: 317 - DNA, 75 bp, Artificial Sequence
  Description of Artificial Sequence Synthetic oligonucleotide
gtgaacatctcccggatctgatccgccgcttttcacaacagcgcgagccgcaggaccgtggagggggcggcaaaa

SEQ ID NO: 318 - DNA, 28 bp, Artificial Sequence
  Description of Artificial Sequence Synthetic probe
gtgaacatctcccggatctgatccgccg

SEQ ID NO: 319 - DNA, 64 bp, Artificial Sequence
  Description of Artificial Sequence Synthetic oligonucleotide
gtgaacatctcccggatctgatccgccgcttttcccaccagcgcgaccggcggatcgttgaggg

SEQ ID NO: 320 - DNA, 125 bp, Human papillomavirus
  modified_base (40)..(40): a, c, t, g, unknown or other
acaattacattatgtacatatacattatgtaattaaaaancnngcatgtgtatgtattaaaaataatggtatataaacaaatacaatatatacaataaaacacctaaacgcagaggctgctgtta

SEQ ID NO: 321 - DNA, 152 bp, Artificial Sequence
  Description of Artificial Sequence Synthetic polynucleotide
agttatggtatacaacaattatatgtaacaattacattatgtacatatacattatgtaattaaaaagcgtgcatgtgtatgtattaaaaataatggtatataaacaaatataatatatacaataaaacacctaaacgcagaggctgctgtta

SEQ ID NO: 322 - DNA, 106 bp, Human papillomavirus
agttatggtatacaacaattatatgtaacaattacattatgtacatatacattatgtaattaaaaagcgtgcatgtgtatgtattaaaaataatggtatataaaca

SEQ ID NO: 323 - DNA, 110 bp, Artificial Sequence
  Description of Artificial Sequence Synthetic polynucleotide
agttatggtatacaacaattatatgtaacaattacattatgtacatatacattatgtaattaaaaagcgtgcatgtgtatgtattaaaaataatggtatataaacaaata

SEQ ID NO: 324 - DNA, 128 bp, Human papillomavirus
tctttaaatactgctaacatagctccttgtttattgtttacttttaacaagtcttttaattgtgctatggtacattgtggatttacattttctatattgctattgtcacttgtaccgtctacactgct

SEQ ID NO: 325 - DNA, 152 bp, Artificial Sequence
  Description of Artificial Sequence Synthetic polynucleotide
tctgtaaatgatagcccatatgtgtctttaaatactgctaacatagctccttgtttattgtttacttttaacaagtcttttaattgtgctatggtacattgtggatttacattttctatattgctattgtcacttgtaccgtctacactgct

SEQ ID NO: 326 - DNA, 109 bp, Human papillomavirus
tctgtaaatgatagcccatatgtgtctttaaatactgctaacatagctccttgtttattgtttacttttaacaagtcttttaattgtgctatggtacattgtggattta

SEQ ID NO: 327 - DNA, 152 bp, Artificial Sequence; identical to SEQ ID NO: 325
  Description of Artificial Sequence Synthetic polynucleotide

SEQ ID NO: 328 - DNA, 106 bp, Artificial Sequence
  Description of Artificial Sequence Synthetic probe
gggttaaataacaaaacattagacgataaaacaagttataaaatcgatggtaaaggttggcaaaaagataaatcttggggtggttacaacgttacaagatatgaag

SEQ ID NO: 329 - DNA, 106 bp, Staphylococcus aureus
gaagtatagaacattgcaacattggtggggttctaaatagaaaaacggttggaaatggtagctaaaatattgaacaaaatagcagattacaaaacaataaattggg

SEQ ID NO: 330 - DNA, 121 bp, Artificial Sequence
  Description of Artificial Sequence Synthetic probe
atttatctttttgccaacctttaccatcgattttataacttgttttatcgtctaatgttttgttatttaacccaatcattgctgttaatattttttgagttgaacctggtgaagttgtaat

SEQ ID NO: 331 - DNA, 121 bp, Staphylococcus aureus; identical to SEQ ID NO: 330

(19) United States
(12) Patent Application Publication
(10) Pub. No.: US 2013/0261196 A1
(43) Pub. Date: Oct. 3, 2013

(54) Nucleic Acids For Multiplex Organism Detection and Methods Of Use And Making The Same

(71) Applicants: Lisa Diamond, Alameda, CA (US); Jochen Kumm, Redwood City, CA (US); Philip Alexander Rolfe, Newton, MA (US)

(72) Inventors: Lisa Diamond, Alameda, CA (US); Jochen Kumm, Redwood City, CA (US); Philip Alexander Rolfe, Newton, MA (US)

(21) Appl. No.: 13/703,489

(22) PCT Filed: Jun. 10, 2011
(86) PCT No.: PCT/US11/40106

§ 371 Date: May 16, 2013

Related U.S. Application Data
(60) Provisional application No. 61/354,011, filed on Jun. 11, 2010.
(60) Provisional application No. 61/374,041, filed on Aug. 16, 2010.
(60) Provisional application No. 61/439,167, filed on Feb. 3, 2011.

(51) Int. Cl. C12Q 1/70; C12Q 1/68; C12N 15/10

(52) U.S. Cl. CPC C12Q 1/708; C12N 15/1089; C12Q 1/701; C12Q 1/689
USPC 514/789; 506/16; 506/9; 506/26

(57) ABSTRACT

The invention provides mixtures of linear nucleic acid probes, including circularizing “capture” probes, capable of massively multiplex capture of one or more sequences of interest from a plurality of target organisms. The methods provided by the invention enable rapid, precise, and economical detection of one or more organisms of interest, such as common pathogens.

The invention is directed to sets of nucleic acid probes for multiplex detection of organisms of interest, including pathogens, and methods of making and using the probes.

Advances in sequencing technology have continued to drive a precipitous decline in per base sequencing costs. The s1,000 personal genome benchmark proposed by the U.S. National Human Genome Research Institute (NHGRI), however, remains elusive. Moreover, even a patient's complete genome provides little or no insight into a patient's current disease state, such as an ongoing infection. Infectious diseases, in turn, can be caused by a wide variety of pathogens, including viruses, bacteria, archaea, fungi, and other eukaryotes (both single cellular and multicellular), many of which can be cultured only with great difficulty or not at all, hindering detection and selection of proper clinical intervention.

A patient's microbiome—the collection of all the microbes present in and on the patient (see, for example, Friedrich MJ, JAMA 300(7):777-8 (2008)—can reveal a patient's current disease state as well as help a caregiver to predict their future risk of disease, infection, or clinical complications. The microbiome, however, is extremely complex, as evidenced by the microbial diversity that can be observed in even a single microenviroment of the human body. See, e.g., Hyman et al., PNAS 102(22):7952-7 (2005) (studying the microbial diversity on the human vaginal epithelium). Existing modalities for organism detection are poorly suited to detecting organisms in complex samples, such as a patient sample, because they are generally limited to single pathogen assays that are expensive and time consuming.

Moreover, existing platforms to design nucleic acid probes for pathogen detection require a single short region of DNA (a few hundred or few thousand bases long) as the input. Accordingly, these platforms offer very limited choices of genomic regions, such as the 16S ribosomal DNA region, to detect and differentiate between organisms and thus fail to identify optimal primer candidates from the widest possible range of sequences. In addition, since existing tests are often based on interrogating only a single target locus of a single target pathogen, these tests often fail to differentiate between closely related species or strain variants of a particular organism, which can vary considerably in their pathogenicity, sensitivity to antibiotics, or production of toxins—factors that will dramatically influence the decisions of a caregiver.

In view of the difficulties of existing assays in detecting organisms of interest in complex sample mixtures and the failure of existing platforms for primer design to identify optimal primer candidates from the widest possible range of sequences, a need exists for rapid, multiplex assays that detect a plurality of organisms in complex mixtures without the need for culturing.

Embodiments of the present invention include optimized nucleic acid probes, and methods of making and using them, that enable the skilled artisan to simultaneously detect a plurality of organisms in a complex mixture, without the need for culturing. The invention is based, at least in part, on the discovery of a process that can rapidly identify sequences from sets of large query sequences, such as whole genomes. The sequences can be used in multiplex diagnostic assays that dramatically reduce assay time and cost, compared to conventional diagnostics. The nucleic acids and methods of the invention enable the skilled artisan to identify the species of an infectious agent(s) and even differentiate between closely related strains based on the sequence of regions associated with, for example, antibiotic resistance.

A further advantage of the methods of the invention is the ability to interrogate specific host loci in parallel with detecting infectious agents, e.g., for host genotyping. Advantageously, the methods of the invention may be further multiplexed and used in automated systems, such as microplates, for high throughput processing of large numbers of samples by centralized laboratory, hospital, and/or diagnostic facilities. Additionally, the mixtures and methods of the invention can be used in a wide variety of additional applications, such as monitoring water supplies, foodstuffs, and agricultural samples.

Accordingly, aspects of the invention provides mixtures comprising a plurality of nucleic acid probes capable of circularizing capture of a region of interest. In some embodiments, the probes in the mixture each comprise a first and second homologous probe sequence—separated by a backbone sequence—that specifically hybridize to a first and second target sequence, respectively, in the genome of at least one target organism. In some embodiments the first and second homologous probe sequences are not complementary to the target sequence, but ligate to the 5′ and 3′ termini of a target nucleic acid, e.g. a microRNA, and possess appropriate chemical groups for compatibility with a nucleic acid-ligating enzyme, such as phosphorylated or adenylated 5′ termini, and free 3′ hydroxyl groups. In some embodiments, the first and second target sequences are separated by a region of interest of at least two nucleotides. In particular embodiments, they are separated by at least 5, 6, 7, 8, 9, 10, 12, 14, 18, 20, 25, 30, 50, 75, 100, 150, 200, 300, 400, 600, 1200, 1500, 2500, or more nucleotides. In some embodiments, the first and second target sequences are separated by no more than 5, 6, 7, 8, 9, 10, 12, 14, 18, 20, 25, 30, 50, 75, 100, 150, 200, 300, 400, 600, 1200, 1500, or 2500 nucleotides.

In some embodiments, the homologous probe sequences in the mixture specifically hybridize to target sequences in the genome of their respective target organism, but do not specifically hybridize to any sequence in the genome of a predetermined set of sequenced organisms—the exclusion set. In embodiments related to probes that do not hybridize directly to the capture target, the ‘homologous probe sequences’ are designed specifically to not substantially hybridize to any sequence within a defined set of genomes, i.e., an exclusion set. In the case of biological samples from a subject, the exclusion set includes the host's genome. In particular embodiments, the exclusion set also includes a plurality of viral, eukaryotic, prokaryotic, and archaeal genomes. In more particular embodiments, the plurality of viral, eukaryotic, prokaryotic, and archaeal genomes in the exclusion set may comprise sequenced genomes from commensal, non-virulent, or non-pathogenic organisms. In still more particular embodiments, the exclusion set for all probes in a mixture share a common subset of sequenced genomes comprising, for example, a host genome and commensal, non-virulent, or non-pathogenic organisms. In general, the exclusion set varies between probes in the mixture so that each probe in the mixture does not specifically hybridize with the target sequence of any other probe in the mixture.

In one aspect, the invention encompasses a plurality of nucleic acid probes each comprising homologous probe sequences which are substantially free of secondary structure, do not contain long strings of a single nucleotide (e.g., they have fewer than 7, 6, 5, 4, 3, or 2 consecutive identical bases), are at least about 8 bases (e.g., 8, 10, 12, 14, 16, 18, 20, 22, 24, 25, 26, 27, 28, 30, or 32 bases in length), and have a Tₘ in the range of 50-72° C. (e.g., about 53, 54, 55, 56, 57, 58, 59, 60, 61, or 62° C.). In some embodiments the first and second homologous probe sequences are about the same length and have the same Tₘ. In other embodiments, length and Tₘ of the first and second homologous probe sequences differ. The homologous probe sequences in each probe may also be selected to occur below a certain threshold number of times in the target organism's genome (e.g., fewer than 20, 10, 5, 4, 3, or 2 times).

The target organism for a particular probe may be any organism. In particular embodiments it may be viral, bacterial, fungal, archaeal, or eukaryotic, including single cellular and multicellular eukaryotes. In particular embodiments the target organism is a pathogen.

The mixtures of the invention can include large number of probes, e.g., 10, 20, 30, 40, 50, 100, 200, 400, 500, 1000, 2000, 3000, 4000, 5000, 10000, 20000, 40000, 80000, or more. The mixture can include one or more probes directed to a large number of different target organisms, e.g., at least 10, 20, 40, 60, 80, 100, 150, 200, 250, or more different target organisms. In some embodiments, a mixture including one or more probes to a plurality of target organisms contains only one probe to a target organism. In other embodiments, the mixture contains more than one probe to a target organism, e.g., about 2, 3, 4, 5, 6, 7, 8, 9, or 10 probes for a target organism. In certain embodiments, such as embodiments designed for use with patient test samples, the mixture further includes probes with homologous probe sequences that specifically hybridize to the host genome for applications such as host genotyping. In some embodiments, the mixtures of the invention further comprise sample internal calibration standards.

The backbone sequence of the probes in the mixtures provided by the invention may include a detectable moiety and a primer-binding sequence. In some embodiments, the backbone sequence of the probes comprises a second primer. In particular embodiments, the detectable moiety is a barcode. In certain embodiments the backbone further comprises a cleavage site, such as a restriction endonuclease recognition sequence. In certain embodiments, the backbone contains non-Watson-Crick nucleotides, including, for example, abasic furan moieties, and the like.

In another aspect, the invention provides a kit comprising a mixture of probes provided by the invention and instructions for use. In particular embodiments, the kit may also comprise reagents for obtaining a sample (e.g., swabs), and/or reagents for extracting DNA, and/or enzymes, such as polymerase and/or ligase to capture a region of interest.

In another aspect, the invention provides a method for detecting the presence of one or more target organisms by contacting a sample suspected of containing at least one target organism with any of the mixtures of probes of the invention, capturing a region of interest of the at least one target organism (e.g., by polymerization and/or ligation) to form a circularized probe, and detecting the captured region of interest, thereby detecting the presence of the one or more target organisms. In certain embodiments, the captured region of interest may be amplified to form a plurality of amplicons (e.g., by PCR). In particular embodiments the sample is treated with nucleases to remove the linear nucleic acids after probe-circularizing capture of the region of interest. In some embodiments, the circularized probe is linearized, e.g., by nuclease treatment. In other embodiments the circularized probe molecule is sequenced directly by any means known in the art, without amplification. In certain embodiments, the circularized probe is contacted by an oligonucleotide that primes polymerase-mediated extension of the molecules to generate sequences complementary to that of the circularized probe, including from at least one to as many as 1 million or more concatemerized copies of the original circular probe. In particular embodiments, the circularized probe molecule is enriched from the reaction solution by means of a secondary-capture oligonucleotide capture probe. A secondary-capture oligonucleotide capture probe may comprise a moiety designed to be captured, such as a biotin molecule, and a nucleic acid sequence designed to hybridize to at least 6 nucleotides of the circularized probe. The nucleic acid sequence designed to hybridize to at least 6 nucleotides of the circularized probe may include 1, 2, 4, 8, 16, 32 or more nucleotides of the polymerase-extended capture product. In certain embodiments, the probe and/or captured region of interest is sequenced by any means known in the art, such as polymerase-dependent sequencing (including, dideoxy sequencing, pyrosequencing, and sequencing by synthesis) or ligase based sequencing (e.g., polony sequencing). In particular embodiments, the sample is a biological sample. In more particular embodiments the biological sample is from a mammal, such as a human.

In some embodiments the methods of detecting the presence of one or more target organisms further comprise the step of formatting the results to facilitate physician decision making by, for example, providing one or more graphical displays.

Accordingly, in another aspect, the invention provides a method of treating a subject suspected of being infected with a pathogen, comprising detecting at least one target organism (e.g., a pathogen) by the methods of the invention and administering a suitable therapeutic treatment based on the at least one organism detected.

A further aspect of the invention provides methods of making the mixtures of probes provided by the invention. The methods comprise providing a reference genome and an exclusion set of genomes. The sequence of the reference genome is sliced (in silico) into n-mer strings of about 18-50 nucleotides. The sliced n-mer strings are screened to eliminate redundant sequences, sequences with secondary structure, repetitive sequences (e.g., strings with more than 4 consecutive identical nucleotides), and sequences with a Tₘ outside of a predetermined range (e.g., outside of 50-72° C.). The screened n-mers are further screened to identify homologous probe sequences by eliminating n-mers that specifically hybridize to a sequence in the genome in the exclusion set of genomes (e.g., if a pairwise alignment contains 19 of 20 matches in an n-mer, such as a 25-mer) or occurs in the genome of the target organism more than a specified number of times. In particular embodiments, a homologous probe sequence occurs only once in the genome of the target organism. For target organisms with a single-stranded genome, the homologous probe sequence may occur only once in the complement of the genome of the target organism. In one embodiment, where a sequenced variant of the target organism is available (e.g., the same species, genus, or serovar), the homologous probe sequences are filtered so as to specifically hybridize to the genome of the additional sequenced variant(s) resulting in a probe that groups related organisms. In an alternate embodiment, the homologous probe sequences may be filtered so as to not specifically hybridize to the genome of the sequenced variant (e.g., the sequenced variant is part of the exclusion set), resulting in a probe that discriminates between related organisms. These filter processes are iterated for each target organism to be detected by the particular mixture. In some embodiments, the candidate homologous probe sequences are screened to eliminate those that will specifically hybridize with other probes in the mixture.

For each target organism, homologous probe sequences are combined into probes designed, for example, to capture regions of interest of a particular size, or in certain embodiments, to capture a predetermined region of interest (such as a region associated with drug resistance, virulence, or toxin production), or, for subject genotyping, to capture a locus in the subject's genome. Regions of interest may be defined by, e.g., directed human input, statistical methods, sequence data mining, literature data mining, or combinations thereof.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1 is a schematic diagram of one exemplary probe provided by the invention.

FIGS. 2 A, 2B, and 2C are diagrams of 3 alternative methods of using probes as described herein to capture a region of interest.

FIG. 3 depicts exemplary strategies for small nucleic acid cloning using probes as described herein.

FIG. 4 is an illustration of particular methods of the invention using conventional primer pairs for PCR amplification.

FIG. 5 shows an exemplary flow chart for methods provided by the invention, including treatment and diagnostic methods.

FIG. 6 is an illustrative display of possible assay results, formatted to inform physician decision making.

FIG. 7 is a flow chart of an exemplary embodiment of a method for probe design.

FIG. 8 depicts a plot of the fraction of a population of homologous probe sequences that exists in duplex form as a function of melting temperature (Tₘ).

FIGS. 9 and 10 depict the effect of melting temperature on the probe's efficiency, as determined by read count at particular melting temperatures.

FIG. 11 is a flow chart of an exemplary embodiment of a method for, inter alia, processing, analyzing, and outputting of sequencing results.

FIG. 12 is a diagram of exemplary embodiment of a system architecture for implementing analysis and formatting of sequencing data.

FIG. 13, including parts A and B, depicts an exemplary workflow for processing of raw FASTQ data from a sequencing machine and quantification against reference genomes.

FIG. 14 depicts an exemplary alignment of sequences obtained from next generation sequencing reads.

FIG. 15 is a schematic illustration of the use of sequence read alignment against a database of reference strains to identify strains in a sample.

FIG. 16 depicts a method of accurate polymorphism modeling and detection by next generation sequencing.

FIG. 17 shows a matrix of which HPV probes (x-axis) detect which HPV strains (y-axis) in a simulation of HPV strain detection using 346 probes and a set of high-risk HPV strains (HPV 16, 18, 31, 33, 35, 39, 45, 51, 52, 56, 58, 59). White areas indicate probes that detect corresponding strains.

FIG. 18 depicts a target matrix for group of 20 HPV probes versus target HPV strain genomes.

FIG. 19 depicts a target matrix expanded to indicate the number and type of SNPs identified by each of 27 specific HPV probes.

FIG. 20 depicts agarose gel-resolved samples of PCR-amplified HPV probe circularizing capture reactions.

FIG. 21 depicts alignments of circularizing capture reaction products and known bacterial genomic sequences.

FIG. 22 depicts agarose gel-resolved samples of PCR-amplified bacteria or bacterial gene-detecting probe circularizing capture reactions.

FIG. 23 depicts an alignment of observed Sanger sequencing reads of PCR-amplified circularized probe with genomic Staphylococcus aureus sequences.

FIG. 24 depicts detection of cDNA reverse transcribed from RNA using five individual molecular inversion probes and amplification for normal Sanger (N) or Next generation sequencing (T, tailed primer) (probes denoted as 198, 256, 292, 293, and 462).

FIG. 25 depicts the proportions of different infectious species detected by probes in four urinary tract infection patient samples.

FIG. 26 depicts comparative circularizing capture protocols performed using a varying number of (i) PCR cycles, (ii) varying lengths of time for gap filling and ligation, and (iii) varying hybridization temperatures.

### DESCRIPTION OF EMBODIMENTS

### 1. Probes

One aspect of the invention provides mixtures of circularizing “capture” probes suitable for sensitive, rapid, and highly specific detection of one or more organisms in complex samples. “Probe” refers to a linear, unbranched polynucleic acid comprising two homologous probe sequences separated by a backbone sequence, where the first homologous probe sequence is at a first terminus of the nucleic acid and the second homologous probe sequence is at the second terminus to the nucleic acid, and where the probe is capable of circularizing capture of a region of interest of at least 2 nucleotides. “Circularizing capture” refers to a probe becoming circularized by incorporating the sequence complementary to a region of interest. Basic design principles for circularizing probes, such as simple molecular inversion probes (MIPs) as well as related capture probes are known in the art and described in, for example, Nilsson et al., Science, 265:2085-88 (1994), Hardenbol et Genome Res., 15:269-75 (2005), Akharas et al., PLOS One, 9:e915 (2007), Porecca et al., Nature Methods, 4:931-36 (2007); Deng et al., Nat. Biotechnol., 27(4):353-60 (2009), U.S. Pat. Nos. 7,700,323 and 6,858,412, and International Publications WO/1999/049079 and WO/1995/022623.

Certain aspects of the invention encompass probes which include two homologous probe sequences, each of which may specifically hybridize to a different target sequence in the genome of a target organism adjacent to a region of interest comprising at least two nucleotides. The probes may further comprise a backbone sequence, which contains a detectable moiety and a primer, between the homologous probe sequences. Typically, the homologous probe sequence at the 3′ end of the probe is termed H1 (or the extension arm) and the homologous probe sequence at the 5′ end of the probe is termed H2 (the ligation or anchor arm). Upon hybridization to the target sites in the genome of interest, the probe/target duplexes are suitable substrates for polymerase-dependent incorporation of at least two nucleotides on the probe (on the extension arm), and/or ligase-dependent circularization of the probes (either by circularizing a polymerase-extended probe or by sequence-dependent ligation of a linking polynucleotide that spans the region of interest).

“Capture reaction” refers to a process where one or more probes contacted with a test sample has undergone circularizing capture of a region of interest, wherein the first and second homologous probe sequences in the probe have specifically hybridized to their respective target sequence in the test sample to capture the region of interest between the first and second target sequences of the probe. “Capture reaction products” refers to the mixture of nucleic acids produced by completing a capture reaction with a test sample. “Amplification reaction” refers to the process of amplifying capture reaction products. An “amplification reaction product” refers to the mixture of nucleic acids produced by completing an amplification reaction with a capture reaction product.

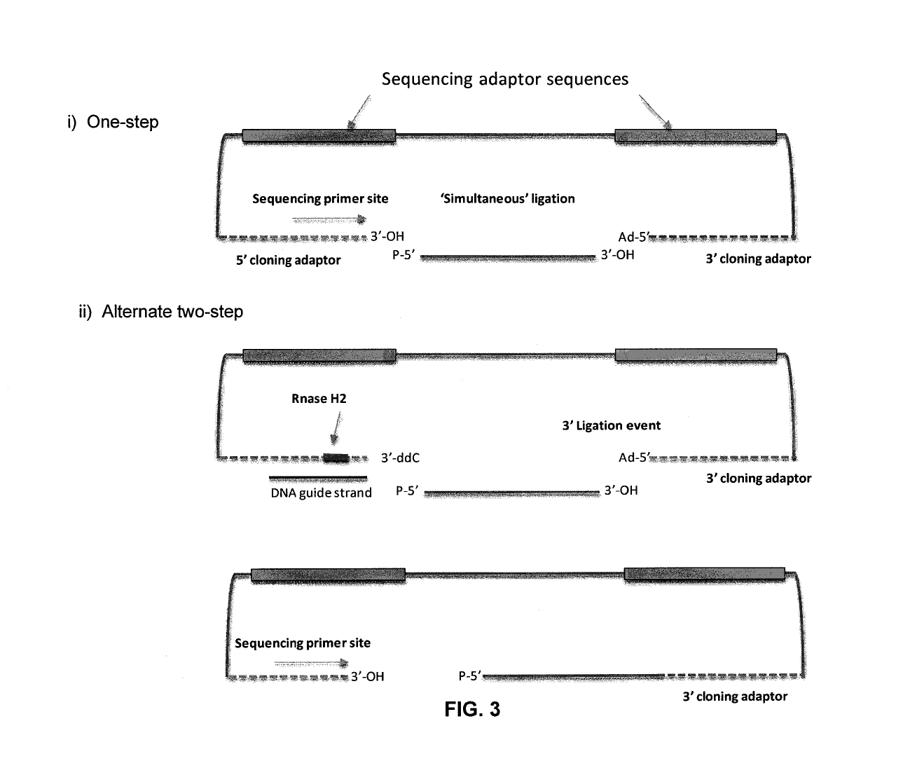

In some embodiments the first and second homologous probe sequences are not complementary to the target sequence, but ligate to the 5′ and 3′ termini of a target nucleic acid, e.g., small RNAs and microRNAs, and possess appropriate chemical groups for compatibility with a nucleic acid-ligating enzyme, such as phosphorylated or adenylated 5′ termini and free 3′ hydroxyl groups. Exemplary strategies for small nucleic acid cloning are shown in FIG. 3. In some embodiments, a probe with an adenylated 5′ end and a free 3′-OH is ligated near-simultaneously to a small RNA fragment containing compatible ligation ends in one step (FIG. 3 (i)). In further embodiments, a probe may capture a small target nucleic acid in a two-step process wherein a probe with an adenylated 5′ end and a blocked 3′ end (e.g., a dideoxy nucleotide-blocked end) may be ligated to the target small RNA (FIG. 3 (ii), first of two probe diagrams in (ii)). This may occur by initial removal of an RNA base within the probe by guided RNase H2 digestion, and subsequent near-simultaneous ligation of the now 3′-OH-terminating probe to the small RNA. In an alternate two-step process, the probe may be ligated to the 5′-adenylated probe site, and then the blocked 3′ end of the probe may be digested by RNase H2 to generate a free 3′-OH for ligation (FIG. 3 (ii), second of two probe diagrams in (ii)).

### 1.1 Homologous Probe Sequences

A “homologous probe sequence” is a portion of a probe provided by the invention that specifically hybridizes to a target sequence present in the genome of an organism of interest. The terms “homologous probe sequence,” “probe arm,” “homer,” and “probe homology region” each refer to homologous probe sequences that may specifically hybridize to target genomic sequences, and are used interchangeably herein. “Target sequence” refers to a nucleic acid sequence on a single strand of nucleic acid in the genome of an organism of interest. In some embodiments, the homologous probe sequences in the probes are each at least 8, 10, 12, 14, 16, 18, 20, 22, 24, 26, 28, 30, 32, 34, 36, 38, 40, 45, 50, 55, 60, 65, 70, 80, 90, 100, 110, 120, or more nucleotides in length. In particular embodiments, the homologous probe sequences are 18-50, 18-36, 20-32, or 22-28 nucleotides in length. In more particular embodiments, the homologous probe sequences are 22-28 nucleotides in length. In certain embodiments, the two homologous probe sequences in a probe are the same length; in other embodiments they are different lengths. In particular embodiments, the homologous probe sequences of a probe differ in length, but by less than 10, 9, 8, 7, 6, 5, 4, 3, or 2 nucleotides.

In some embodiments, homologous probe sequences do not contain long stretches of consecutive identical nucleotides. In some embodiments, homologous probe sequences contain fewer than 10, 9, 8, 7, 6, 5, 4, or 3 consecutive identical nucleotides. In more particular embodiments, they contain fewer than 6 consecutive identical nucleotides, and in more particular embodiments they contain fewer than 4 consecutive identical nucleotides.

Homologous probe sequences may be substantially free of secondary structure, such as hairpins. A homologous probe sequence is “substantially free of secondary structure” when no n-mer of the reverse complement of the homologous probe sequence is perfectly complementary to an n-mer in the homologous probe sequence at least 5 bases away, where n is 7. In some embodiments, n is 15, 14, 13, 12, 11, 10, 9, 8, 6, 5, 4, or 3. In particular embodiments, n is 3-7. In some embodiments, a sequence, e.g., homologous probe sequence, backbone sequence, or probe, is substantially free of secondary structure when less than 30% of the molecules in aqueous solution are in a stable intramolecular hairpin or intermolecular dimer at a concentration of 0.25 μM, with 50 mM Na⁺, and no Mg⁺⁺, at the melting temperature (Tₘ) of the sequence, wherein the solution is free of other sequences. In some embodiments, a sequence is substantially free of secondary structure when less than 30% of the molecules are in a stable intramolecular hairpin or intermolecular dimer at a DNA concentration of 0.25 μM, with 50 mM Na⁺, with no Mg⁺⁺, at 15, 10, 8, 6, 4, or 2° C. below the Tₘ of the sequence, wherein the solution is free of other sequences. In some embodiments, a sequence is substantially free of secondary structure when less than 30% of the molecules are in a stable intramolecular hairpin or intermolecular dimer at a DNA concentration of 0.25 μM, with 50 mM Na⁺ and 0.5 mM Mg⁺⁺, at 15, 10, 8, 6, 4, or 2° C. below the Tₘ of the sequence in the presence of 0.5 mM Mg⁺⁺. Other methods of detecting secondary structure are known in the art, may be used in the present invention, and are described in, for example, Zuker, Nucleic Acids Res., 31:3406-15 (2003); Mathews et al., J. Mol. Biol., 288:911-940 (1999); Hilbers, et al., Anal. Chem. 327:70 (1987); Serra et al., Nucleic Acids Res., 21:3845-3849 (1993); and Vallone et al., Biopolymers., 50: 425-442 (1999).

In some embodiments, the homologous probe sequences are designed to have a melting temperature (Tₘ) of 50-72° C. in the presence of 0.5 mM Mg⁺⁺ e.g., about 50, 52, 54, 56, 57, 58, 59, 60, 61, 62, 63, 64, 65, 66, 67, 68, 69, 70, 71, or 72° C. In particular embodiments, the Tₘ is 50-65° C. in the presence of 0.5 mM Mg⁺⁺. In some embodiments, the Tₘ is 38-72° C. in the absence of Mg⁺⁺. In particular embodiments, the homologous probe sequences in a probe have approximately the same Tₘ, while in other embodiments they have different Tₘs but are within 10, 9, 8, 7, 6, 5, 4, 3, 2, or 1° C. of each other. In certain embodiments the first homologous probe sequence (i.e., the 5′-most in the probe) has a lower Tₘ than the second homologous probe sequence; in other embodiments it has a higher Tₘ than the second homologous probe sequence.

“Melting temperature” (“Tₘ”) refers to the temperature at which 50% of DNA molecules in a solution are hybridized as duplexes with their complementary sequence and half are dissociated. Unless otherwise indicated, Tₘ is determined at a DNA concentration of 0.25 μM and a sodium concentration of 50 mM, with no Mg⁺⁺. Tₘ may be determined by a variety of methods known to the skilled artisan, including empirical measurements or estimation. In certain embodiments, Tₘ is estimated by counting the number or percentage of G and C nucleotides in a sequence. In particular embodiments, the number of G and C nucleotides in a homologous probe sequence is between 30-60% of nucleotides in the sequence, such as about 30, 35, 40, 45, 50, or 55%. In more particular embodiments the number of G and C nucleotides in a homologous probe sequence is 38-44% of nucleotides in the homologous probe sequence.

In particular embodiments, a nearest neighbor estimate of Tₘ, which accounts for base stacking between adjacent nucleotides, is used. Nearest neighbor calculations are described in, for example, Breslauer et al., PNAS, 83: 3746-3750 (1986) and reviewed in SantaLucia, PNAS, 95(4):1460-65 (1998) (reviewing several empirical nearest neighbor studies and providing, inter alia, ΔH and ΔS master table for DNA/DNA duplexes in Table 2), which are incorporated herein by reference.

Homologous probe sequences may be designed to specifically hybridize to target sequences in the genome of the target organism. The term “hybridizes” refers to sequence-specific interactions between nucleic acids by Watson-Crick base-pairing (A with T or U and G with C). “Specifically hybridizes” means a nucleic acid hybridizes to a target sequence with a Tₘ of not more than 8° C. below that of a perfect complement to the target sequence. In certain embodiments, a sequence specifically hybridizes to a target sequence with a Tₘ of not more than 7, 6, 5, 4, 3, 2, or 1° C. below that of a perfect complement to the target sequence. In some embodiments, a sequence specifically hybridizes to a target sequence when it is a perfect complement to a target sequence. In other embodiments a sequence specifically hybridizes to a target sequence when it is about 99, 98, 97, 96, 95, 94, 93, 92, 91, 90, 85, 80, 75, 70, or 65% identical to a perfect complement of a target sequence. In some embodiments, a homologous probe sequence specifically hybridizes to a target sequence but contains mismatches, e.g., about 1, 2, 3, 4, 5, or more mismatches in a window of about 18, 20, 22, 24, 25, 26, 28, 30, 35, 40, or 45 consecutive bases.

In particular embodiments, the probe may hybridize to a nucleic acid sequence that has been appended to a DNA or RNA component or that has been appended to a sequence complementary to a DNA or RNA component of the target genome. Such appended nucleic acid sequences include, for example, an oligonucleotide adapter appended via ligation or a polynucleotide run (for example, “AAAAA” or “CCCCC”) generated by polymerase or nucleotide terminal transferase activity.

In further particular embodiments, a bridge nucleic acid may be employed, wherein at least a first portion of the bridge nucleic acid is capable of hybridizing to the capture probe, and at least a second portion of the bridge nucleic acid (which may overlap with the first portion) is capable of simultaneously or sequentially hybridizing to the target nucleic acid, thereby enhancing the efficiency of ligation of the capture probe to the target.

In particular embodiments, a probe specifically hybridizes when: a) both homologous probe sequences in the probe hybridize to their respective target sequence with at least 60, 65, 70, 75, 80, 85, 90, 95, or 100% correct pairing across the entire length of the homologous probe sequence; b) the first homologous probe sequence hybridizes with 100% correct pairing in the 8, 7, 6, 5, 4, 3, or 2 bases at the 3′ end of the H1 (3′ most second homologous probe sequence); and c) the second homologous probe sequence hybridizes the first 8, 7, 6, 5, 4, 3, or 2 bases of the 5′ end of the H2 (5′ most homologous probe sequence). In still more particular embodiments, a probe specifically hybridizes when: a) both homologous probe sequences in the probe hybridize to their respective target sequence with at least 80% correct pairing across the entire length of the homologous probe sequence, b) the first homologous probe sequence hybridizes with 100% correct pairing of the first 6 bases of the 3′ end of the H1; and c) the second homologous probe sequence hybridizes with 100% correct pairing of the first 6 bases of the 5′ end of the H2.

Homology between two sequences, e.g., a homologous probe sequence and the complement of a target sequence, may be determined by any means known in the art, including pairwise alignment, dot-matrix, and dynamic programming, and in particular embodiments by FASTA (Lipman and Pearson, Science, 227: 1435-41 (1985) and Lipman and Pearson, PNAS, 85: 2444-48 (1998)), BLAST (McGinnis & Madden, Nucleic Acids Res., 32:W20-W25 (2004) (current BLAST reference, describing, inter alia, MegaBlast); Zhang et al., J. Comput. Biol., 7(1-2):203-14 (2000) (describing the “greedy algorithm” implemented in MegaBlast); Altschul et al., J. Mol. Biol., 215:403-410 (1990) (original BLAST publication)), Needleman-Wunsch (Needleman and Wunsch, J. Molec. Bio., 48 (3): 443-53 (1970)), Sellers (Sellers, Bull. Math. Biol., 46:501-14 (1984), and Smith-Waterman (Smith and Waterman, J. Molec. Bio., 147: 195-197 (1981)), and other algorithms (including those described in Gerhard et al., Genome Res., 14(10b):2121-27 (2004)), which are incorporated herein by reference. In particular embodiments, the methods provided by the invention comprise screening candidate sets of sequences by MegaBLAST against one or more annotated genomes.

In some embodiments, a sequence “specifically hybridizes” when it hybridizes to a target sequence under stringent hybridization conditions. “Stringent hybridization conditions” refers to hybridizing nucleic acids in 6×SSC and 1% SDS at 65° C., with a first wash for 10 minutes at about 42° C. with about 20% (v/v) formamide in 0.1×SSC, and a subsequent wash with 0.2×SSC and 0.1% SDS at 65° C. In particular embodiments, alternate hybridization conditions can include different hybridization and/or wash temperatures of about 55, 56, 57, 58, 59, 60, 61, 62, 63, 64, 66, 67, 68, 69, or 70° C. or other hybridization conditions as disclosed in Sambrook and Russell, Molecular Cloning: A Laboratory Manual, Cold Spring Harbor Laboratory Press, 3rd edition (2001), which is incorporated herein by reference. In particular embodiments, the hybridization temperature is greater than 60° C., e.g., 60-65° C.

Homologous probe sequences may be selected to specifically hybridize to a target sequence in the genome of a particular organism or, in particular embodiments, the genomes of a group of closely related organisms. Accordingly, in some embodiments, a homologous probe sequence does not specifically hybridize to a sequence contained in an exclusion set of sequenced genomes. “Exclusion set” refers to a predetermined set of sequenced genomes to which a homologous probe sequence does not specifically hybridize. In embodiments encompassing probes that do not hybridize directly to the capture target, the homologous probe sequences are designed specifically to not substantially hybridize to any sequence within the exclusion set. In some embodiments, a homologous probe sequence contains at least 1, 2, 3, 4, 5, 6, 7, 8, 9, or 10 mismatches in a window of about 15, 20, 21, 22, 23, 24, 25, 26, 27, 28, 29, 30, 35, or 40 consecutive bases to a sequence in the exclusion set. In more particular embodiments the homologous probe sequences in a probe each have at least one mismatch in 20 bases to any sequence in the exclusion set.

An “organism” is any biologic with a genome, including viruses, bacteria, archaea, and eukaryotes including plantae, fungi, protists, and animals.

A “sequenced organism(s)” is an organism where a sufficient portion of its genome has been sequenced to be able to differentiate it from other organisms. A “sequenced genome” or “or “genome of sequenced organism(s)” is the nucleotide sequence of a sequenced organism's genome. In some embodiments, the sequenced organism is fully or partially sequenced (e.g., by shotgun or cDNA sequencing, library sequencing, BAC or YAC sequencing). In particular embodiments, the organism's genome is at least 5, 10, 20, 30, 40, 50, 60, 70, 80, 90, 95, or 99% sequenced. Sequenced genomes may be sequenced at a variety of levels of coverage, such as about 0.1, 0.5, 0.8, 1, 2, 3, 4, 5, 10, 20×, or more, coverage. In some embodiments, genome sizes for organisms of interest, such as pathogens, may be at least 0.01, 0.05, 0.1, 0.5, 1, 2, 3, 4, 5, 6, 7, 8, 9, 10, 20, 50, 100, 200, 500, 1000 million bases, or more. In particular embodiments target genomes are at least 0.01 to 10 million bases.

In particular embodiments, the exclusion set comprises a genome of the subject organism from which a test sample is obtained. In certain embodiments, the exclusion set comprises a human genome. In more particular embodiments the exclusion set further comprises the genomes of common human microflora or commensal organisms. In still more preferred embodiments, the exclusion set further comprises the genomes of the target organism for other probes in a mixture, e.g., a panel (e.g., so that only one probe in a mixture specifically hybridizes to any given target organism). In some embodiments, the exclusion set may also comprise a plurality of viral, eukaryotic, prokaryotic, and archaeal genomes. In more particular embodiments, the plurality of viral, eukaryotic, prokaryotic, and archaeal genomes in the exclusion set may further comprise sequenced genomes from commensal, non-virulent, or non-pathogenic organisms. In still more particular embodiments, the exclusion set further comprises sequenced genomes of organisms other than the target organism, including sequenced pathogens. In some embodiments, the exclusion set for all probes in a mixture share a common subset of sequenced genomes comprising, for example, a host genome and commensal, non-virulent, or non-pathogenic organisms. In further embodiments, the exclusion set varies between probes in a mixture so that each probe in the mixture does not specifically hybridize with either the target regions or homologous probe sequences of any other probe in the mixture.

The probes provided by the invention may include a first and second homologous probe sequence that specifically hybridize to a first and second target sequence in the genome of an organism of interest. The first and second target sequence are separated by a region of interest comprising at least 2, 3, 4, 5, 6, 7, 8, 9, 10, 15, 20, 30, 40, 50, 60, 80, 100, 125, 150, 200, 250, 300, 350, 400, 500, 600, 700, 800, 900, 1000, 1200, 1400, 1600, 1800, or 2000 nucleotides. “Region of interest” refers to the sequence between the nearest termini of the two target sequences of the homologous probe sequences in a probe. In certain embodiments, particular target regions may be selected based on human input or computational data mining, including statistical sequence and/or literature data mining. In certain particular embodiments, one or more regions of interest are polymorphic between closely related organisms (e.g., between species of the same genus; between subspecies of the same species; or between strains of the same species or subspecies). In more particular embodiments, the polymorphisms are associated with drug resistance, toxin production, or other virulence factors. In still more particular embodiments, a region of interest includes one or more of those disclosed in, for example, Arnold, Methods Mol. Biol., 642:217-23 (2010) (discussing the RNA polymerase B gene, associated with rifampicin sensitivity in multidrug-resistant (MDR) strains of M. tuberculosis); Kurt et al., J. Clin Microbiol., 47:577-85 (2009) (genotyping regions of S. aureus associated with methicillin resistance); Akhras et al., PLOS ONE, 2(9) e915 (2007) (describing regions from N. gonorrhoeae associated with resistances to ciprofloxacin), and Pourmand et al., PLoS One., 1(1):e95. (2006) (describing a rapid assay for H5N1 virus; identifying cleavage site, glycosylation sites on hemagglutinin gene; oseltamivir resistance site on neuraminidase).

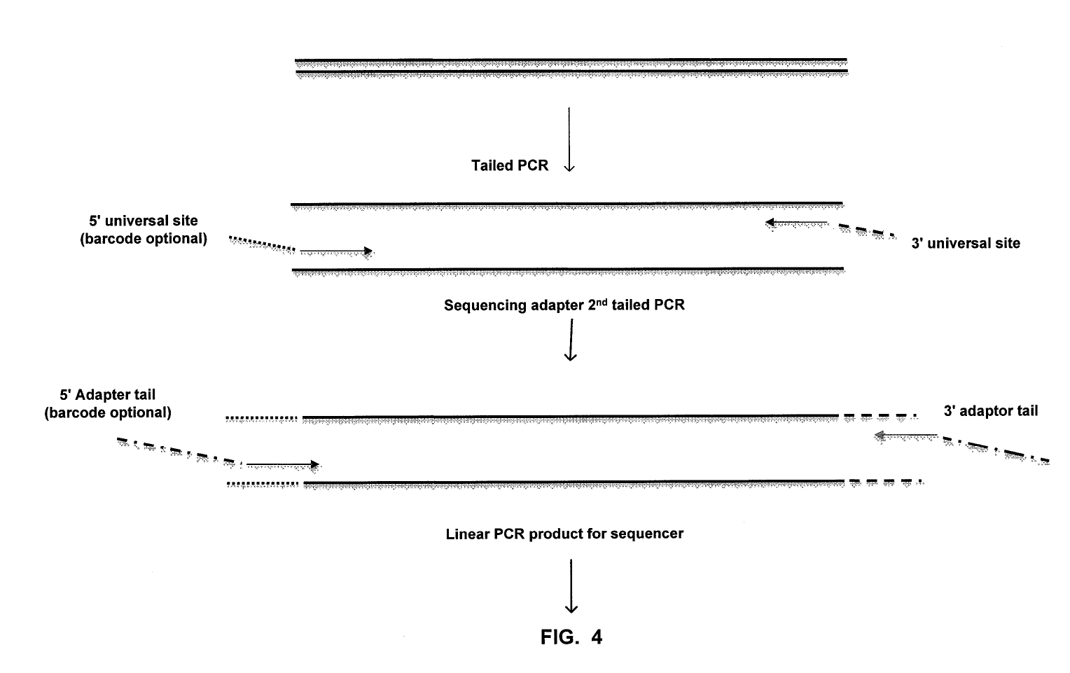

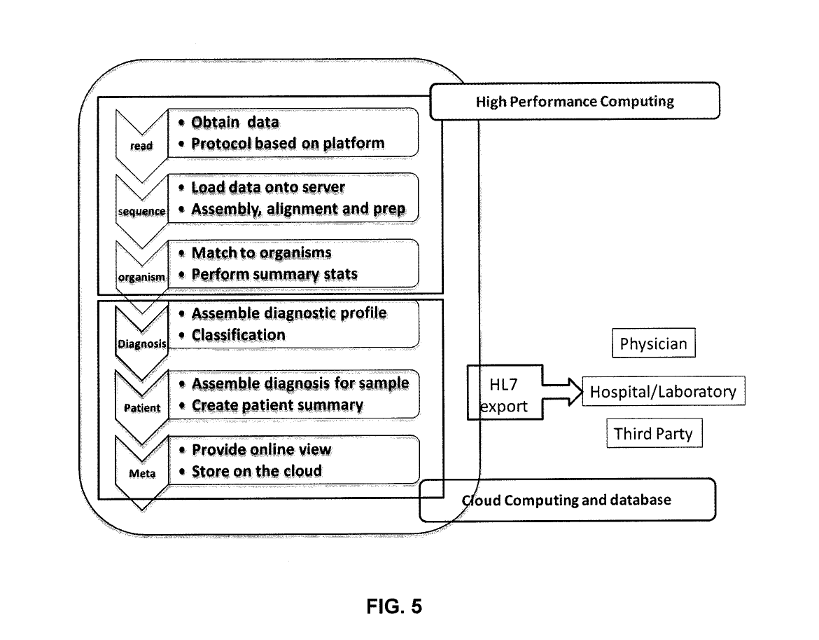

The first and second homologous probe sequences in a probe provided by the invention can readily be adapted for use as a pair of conventional primer pairs for use in a polymerase chain reaction (PCR) to specifically amplify a region of interest from an organism of interest. “Conventional primer pairs” refers to a pair of linear nucleic acid primers each member of which comprises sequences corresponding to one of the two homologous probe sequences in a probe provided by the invention, which are capable of exponential amplification of a region of interest comprising at least two nucleotides. These conventional primer pairs are encompassed by and are a part of the present invention. Accordingly, conventional primer pairs provided by the invention are characterized by the same criteria provided above for homologous probe sequences, including, for example, length, Tₘ, hybridization specificity, and length of the intervening region of interest. In contrast to the probes provided by the invention, which are capable of circularizing capture of a sequence complementary to a region of interest, conventional primer pairs are oriented with their 3′ ends facing each other to facilitate exponential amplification. FIG. 4 is an illustration of particular methods of the invention using conventional primer pairs. In certain embodiments, the conventional primer pairs comprise a barcode sequence. In some embodiments, the conventional primer pairs comprise universal sequences, including, for example, sequences that hybridize to adaptamer primers.

The probes and conventional primer pairs provided by the invention may comprise the naturally occurring conventional nucleotides A, C, G, T, and U (in deoxyriobose and/or ribose forms) as well as modified nucleotides such as 2′O-Methyl-modified nucleotides (Dunlap et al, Biochemistry. 10(13):2581-7 (1971)), artificial base pairs such as IsodC or IsodG, or abasic furans (such as dSpacer) (Chakravorty, et al. Methods Mol. Biol. 634:175-85 (2010)), that do not form canonical Watson-Crick hydrogen bonds), biotinylated nucleotides, adenylated nucleotides, nucleotides comprising blocking groups (including photocleavable blocking groups), and locked nucleic acids (LNAs; modified ribonucleotides, which provide enhanced base stacking interactions in a polynucleic acid; see, e.g., Levin et al. Nucleic Acid Res. 34(20):142 (2006)), as well as a peptide nucleic acid backbone. In particular embodiments, the 5′ or 3′ homologous probe sequences of a probe provided by the invention comprise, at their respective termini, a photocleavable blocking group, such as PC-biotin. In more particular embodiments, a probe provided by the invention comprises a photocleavable blocking group at its 5′ terminus to block ligation until photoactivation. In other particular embodiments, a probe provided by the invention comprises at it's 3′ terminus a photocleavable blocking group to block polymerase-dependent extension or n-mer oligonucleotide ligation until photoactivation.

In other embodiments, the 5′-most nucleotide of a probe provided by the invention comprises an adenylated nucleotide to improve ligation and/or hybridization efficiency. In other embodiments, the homologous probe regions comprise one or more 2′OMethyl, artificial base pairs such as IsodC or IsodG, or abasic furans (such as dSpacer), or 2′OMethyl, abasic furans, or LNA nucleotides, e.g., 1, 2, 3, 4, 5, 6, 7, 8, 9, 10, or more LNAs or 10, 20, 30, 40, 50, 60, 70, 80, 90, or 100% 2′OMethyl, abasic furans, or LNA nucleotides, to improve hybridization and/or ligation efficiency, or provide resistance to enzymatic activities such as polymerase-mediated strand displacement or nuclease cleavage. See, e.g., Hogrefe et al, J. Chem. 265 (10): 5561-5566, (1990). In more particular embodiments, the 5′ end of the 5′ homologous probe region (e.g., H2, the ligation arm) comprises at least one LNA and in still more particular embodiments, the 5′ terminal nucleotide is a LNA.

### 1.2 Backbone Sequences

The probes provided by the invention include a probe backbone sequence between the first and second homologous probe sequences that may include a detectable moiety and one or more primer-binding sequences. The backbone sequence can be at least 15, 20, 25, 30, 35, 40, 45, 50, 70, 90, 100, 12, 140, 150, 160, 180, 200, 400 bases, or more. In more particular embodiments, the backbone includes a second primer. Each backbone primer may comprise one or more universal sequences that, for example, can be used to amplify all circularized probes in a mixture. In some embodiments, the primers may also contain probe-specific sequences, such as barcodes, for identification and/or amplification of a specific probe or set of probes. In some embodiments, the backbone sequence comprises one or more non Watson-Crick nucleotides. In further embodiments, the backbone comprises one or more 2′OMethyl nucleotide residues, artificial base pairs such as IsodC or IsodG, or abasic furans (such as dSpacer), or 2′OMethyl, abasic furans, or LNA nucleotides, e.g., 1, 2, 3, 4, 5, 6, 7, 8, 9, 10, or more LNAs or 10, 20, 30, 40, 50, 60, 70, 80, 90, or 100% 2′OMethyl, abasic furans, or LNA nucleotides, to confer greater reactivity or inertness in the hybridization reaction, provide resistance to enzymatic activities such as polymerase-mediated strand displacement or nuclease cleavage, to serve as inhibitors of spurious amplification events, or to act as target sites for trans-acting nucleic acid oligonucleotides such as PCR primers or biotinylated capture probes.

The term “barcode” is used to refer to a nucleotide sequence that uniquely identifies a molecule or class of related molecules. Suitable barcode sequences for use in the probes of the invention may include, for example, sequences corresponding to customized or prefabricated nucleic acid arrays, such as n-mer arrays as described in U.S. Pat. No. 5,445,934 to Fodor et al. and U.S. Pat. No. 5,635,400 to Brenner. In certain embodiments, the n-mer barcode may be at least 3, 4, 5, 6, 7, 8, 9, 10, 12, 15, 16, 17, 18, 19, 20, 21, 22, 23, 24, 25, 30, 35, 40, 50, 60, 70, 80, 90, 100, 200, 300, 400 or 500 nucleotides, e.g., from 18 to 20, 21, 22, 23, 24, or 25 nucleotides. In particular embodiments the barcodes include sequences that have been designed to require greater than 1, 2, 3, 4 or 5 sequencing errors to allow this barcode to be inadvertently read as another in error.

To generate barcode sequences, for each barcode size K, 4^{K} random barcodes may be generated from the four DNA nucleotides, A,T,G,C, using a pert script. This set of barcodes represents the total number of unique sequence combinations possible for a sequence of K length, using 4 nucleotide variations. Barcodes for which one nucleotide comprises 100% of the length, e.g., TTTTTT, are then optionally removed using a pattern-matching pert script. Further filtering steps may include removal of barcodes which contain runs of nucleotides of >3, e.g., TGGGGT, or runs interrupted by only one nucleotide, for instance, GGGTGG. Barcodes containing palindromes or inverted repeats with a propensity to form secondary structure through self-hybridization may be filtered using a pert script designed to identify such self-complmentarity.

Selection of barcodes that may be utilized in a mixture of probes used to test a sample from a patient may involve selecting a combination of barcodes that will provide >5% and not more than 50% representation of a particular nucleotide at each position in the barcode sequence within the pool. This is achieved by random addition and removal of barcodes to a pooled set until the conditions specified are met using a perl script. Barcodes for which the reverse complement sequence is also present within the barcode pool may also be eliminated.

Suitable barcode sequences include such barcode sequences as set forth in Table 1, which illustrates exemplary 3-mer, 4-mer, 5-mer, 6-mer, 7-mer, 8-mer, 9-mer, and 10-mer barcode sequences. Sequences indicated as “1 nucleotide distance” n-mers in Table 1 are illustrative sequences that have a sequence distance of at least 1 from each other, where “distance” refers to the minimum number of sequencing differences between each of the sequences of the same category. “Two nucleotide distance” sequences have a “distance” from each other of at least 2 nucleotides.

TABLE 1
Exemplary barcode sequences

3-mer barcode-1 nucleotide distance
aaa SEQ ID NO: (add below)
aac
aag
aat
aca
acc
3-mer barcode-2 nucleotide distance
acg
aga
atc
cag
ccc
cgt
4-mer barcode-1 nucleotide distance
aaaa
aaac
aaag
aaat
aaca
aacc
4-mer barcode-2 nucleotide distance
aagg
aatt
acat
accg
acgc
acta
5-mer barcode-1 nucleotide distance
aaaaa
aaaac
aaaag
aaaat
aaaca
aaacc
6-mer barcode-1 nucleotide distance
aaaaaa
aaaaag
aaaaat
aaaaca
aaaact
aaaaga
7-mer barcode-1 nucleotide distance
aaaaaaa
aaaaaac
aaaaaag
aaaaaat
aaaaacg
aaaaagc
8-mer barcode-1 nucleotide distance
aaaaaaaa
aaaaaaat
aaaaaaga
aaaaaatg
aaaaagcg
aaaaatct
9-mer barcode-1 nucleotide
aaaaaaaaa
aaaaaaaac
aaaaacggg
aaaaagagg
aaaaaggac
aaaaattgc
10-mer barcode-1 nucleotide distance
aaaaaactgg
(SEQ ID NO: 1)
aaaaaagcat
(SEQ ID NO: 2)
aaaaaatatc
(SEQ ID NO: 3)
aaaaacactc
(SEQ ID NO: 4)
aaaaactttg
(SEQ ID NO: 5)
aaaaagggtt
(SEQ ID NO: 6)

In particular embodiments, barcodes used in the probes provided by the invention correspond to those on the Tag3 or Tag4 barcode arrays by AFFYMETRIX™. Further discussion of barcode systems can be found in Frank, BMC Bioinformatics, 10:362 (2009; 13 pages), Pierce et al., Nature Methods, 3: 601-03 (2006) (including web supplements), and Pierce et al., Nature Protocols, 2: 2958-74 (2007).

In some embodiments, the backbone comprises one or more sample nucleic acid-specific barcodes, e.g., one or more patient-specific barcodes. In particular embodiments, more than one barcode will be assigned per patient sample, allowing replicate samples for each patient to be performed within the same sequencing reaction. By using sample nucleic acid-specific barcodes it is possible to both multiplex reactions as described in the present application, as well as detect cross-contamination between test samples that did not use a defined repertoire of specific barcodes. In certain embodiments, the backbone may also comprise a temporal barcode, e.g., a barcode that specifies a particular period of time. By using a temporal barcode, it is possible to detect carry-over or contamination on an assay instrument, such as a sequencing instrument, between runs on different days. In more specific embodiments, sample and/or temporal barcodes may be used to automatically detect cross-contamination between samples and/or days and, for example, instruct an instrument operator to clean and/or decontaminate a sample handling system, such as a sequencing instrument.

In certain embodiments, a barcode sequence is also a primer-binding sequence. In some embodiments the backbone primer includes both universal and probe-specific sequences. In some embodiments, the universal sequence is internal (i.e., 3′) to probe-specific regions; in other embodiments, universal sequence(s) is external (i.e., 5′ to probe specific regions). In some embodiments, universal and probe-specific sequences are adjacent. In other embodiments, they are separated by at least 1, 2, 3, 4, 5, 6, 7, 8, 9, 10, 15, 20, 25, 30, 40, or 50 nucleotides, or more.

In certain embodiments, universal primer sequences in a backbone sequence serve as a hybridizing template for longer “adaptamer” primers. An “adaptamer primer” is a primer that hybridizes to universal primer sequences in a capture reaction product to facilitate amplification of the capture reaction product and further comprise a sample-specific barcode sequence, e.g., sequence 5′ to the universal primer hybridizing region of the adaptamer primer. Adaptamer primers can be used, for example, to incorporate sample-specific barcodes on amplification reaction products to allow further multiplexing of samples after completing a capture reaction and an amplification reaction. The addition of sample-specific barcodes allows multiple capture and/or amplification reaction products to be pooled before detection by, for example, sequencing. In more particular embodiments, the adaptamer primers further include universal sequences that hybridize to a sequencing primer.

The detectable moiety may be associated with the backbone sequence. It may be bound to the polynucleotide sequence, as in the case of direct labels, such as fluorescent (e.g., quantum dots, small molecules, or fluorescent proteins), chemical or protein-based labels. Alternatively, the detectable moiety may be incorporated within the polynucleotide sequence, as in the case of nucleic acid labels, such as modified nucleotides or probe-specific sequences, such as barcodes. Quantum dots are known in the art and are described in, e.g., International Publication No. WO 03/003015. Means of coupling quantum dots to biomolecules are known in the art, as reviewed in, e.g., Mednitz et al., Nature Materials 4:235-46 (2005) and U.S. Patent Publication Nos. 2006/0068506 and 2008/0087843, published Mar. 30, 2006 and Apr. 17, 2008, respectively.

### 2 Probe Mixtures

### 2.1 Probes and Calibration Standards

The present invention is based, in part, on providing collections of probes that may specifically hybridize to a target sequence in the genome of a target organism (or group of organisms related by, for example, species, genus, or serovar), and do not specifically hybridize to any sequence in an exclusion set, e.g., at least one non-hybridizing genome (such as the host genome and/or a predetermined set of organisms distinct from the target organism, such as an annotated database of sequenced bacterial, viral, eukaryotic, and archaeal organisms, including pathogenic organisms, but not the target organism or group of target organisms).

Aspects of the invention provides mixtures of probes for multiplex analysis of test samples, such as pathogen detection in a biological sample from a patient. The mixtures provided by the invention comprise at least 2, 3, 4, 5, 6, 7, 8, 9, 10, 20, 30, 40, 60, 80, 100, 200, 250, 500, 1000, 2000, 4000, 8000, 10000, 20000, 30000, 40000, 50000, 60000, 70000, 80000, 90000, or 100000 probes. In some embodiments, the mixtures are designed to capture a plurality of sequences from a particular organism. In certain embodiments the mixtures can capture at least one sequence for each of at least 2, 3, 4, 5, 6, 7, 8, 9, 10, 15, 20, 30, 40, 60, 80, 100, 150, 200, 250, 300, 400, 500, 1000, 2000, 4000, 8000, 10000, 15000, or 20000 different target organisms. In particular embodiments, a mixture comprises at least 1, 2, 3, 4, 5, 6, 7, 8, 9, 10, 15, 20, 25, 30, 35, 40, 45, 50, 60, 65, 70, 75, or 80 homologous probe sequence from any one of Tables 4, 6, 8, 10, 11, or the particular sequences mtb-37rv-inha-pr-01-H1, mtb-H37Rv-rpoB-pr-01-H1, mtb-H37Rv-rpoB-pr-01-H2, mtb-H37Rv-rpoB-pr-02-H1, mtb-H37Rv-rpoB-pr-02-H2, or mtb-37rv-inha-pr-01-H2, and combinations thereof. In particular embodiments, the mixture comprises at least 1, 2, 3, 4, 5, 6, 7, 8, 9, 10, 15, 20, 25, 30, 35, 40, 45, 50, 60, 65, 70, 75, or 80 probes comprising the homologous probe sequence pairs listed in any of Tables 4, 6, 8, 10, and 11.

Probes in a mixture will typically have similar bulk properties (such as, homologous probe sequence length, homologous probe sequence Tₘ, and length of the captured region of interest, and the lack of secondary structure) or fall in ranges of similar values. In some embodiments, the Tₘ of the homologous probe sequences in a mixture of probes will be within 10, 9, 8, 7, 6, 5, 4, 3, 2, or 1° C. of each other, or in particular embodiments have the same Tₘ. In some embodiments, the homologous probe sequences in a mixture of probes will all be within 10, 9, 8, 7, 6, 5, 4, 3, 2, or 1 nucleotide in length of each other, and in particular embodiments they are the same length. The length of the region of interest between the target sequences of a probe may be common to all probes in the mixture, or vary over a range of values, such as 2-20, 20-100, 20-200, 40-300, 100-300 nucleotides. In particular embodiments, the regions of interest are within 100, 90, 80, 70, 60, 50, 40, 30, 20, or 10 nucleotides in length of each other. In more particular embodiments, the regions of interest are the same length. Barcode lengths may also vary, but are generally within 25, 20, 15, 10, or 5 nucleotides of each other. In particular embodiments, the barcodes are the same length.

In some embodiments, mixtures provided by the invention comprise capture reaction products and amplification reaction products from different test samples, as further described below. Briefly, different capture reaction products and/or amplification reaction products can be combined and multiplexed before detection, i.e., for concurrent detection. This is accomplished using barcode sequences that identify the test samples. For example, capture reaction products from test sample A will include a sample A-specific barcode and capture reaction products from sample B will include a sample B-specific barcode. When capture reaction products from sample A and sample B are combined for sequencing, all sequences in the sample A capture reaction products are identified by the presence of the sample A-specific barcode sequence.

In certain embodiments, the mixtures of the invention contain sample internal calibration nucleic acids (SICs). In particular embodiments, known quantities of one or more SICs are included in a mixture provided by the invention. In particular embodiments, at least 1, 2, 3, 4, 5, 6, 7, 8, 10, 15, 20, 25, or 30 different SICs are included in the mixture. In particular embodiments, there are about 4 different SICs in a mixture. In some embodiments, the SICs have a nucleotide composition characteristic of pathogenic DNA targets and are present in specific molar quantities that allow for reconstruction of a calibration curve for quality control, e.g., for the processing and sequencing steps for each individual test sample. In certain embodiments, the SICs makes up approximately 10% (molar quantity) of nucleic acids in a mixture, for example, 2, 4, 6, 8, 10, 12, 14, 16, 18, or 20% (molar) of nucleic acids in the mixture. In particular embodiments different SICs are present in different concentrations, for example, in a dilution series, over a 1, 2, 3, 4, 5, 6, 7, 8, 9, 10, 20, 50, 100, 200, 500, 1000, 5000, 10000, 50000, or 100000-fold concentration range from the most dilute to most concentrated SICs in 2, 3, 4, 5, 6, 7, 8, 9, 10, 20, 30, 40, or 50 steps. In particular embodiments, SICs are present in a sample (e.g., a mixture of probes and a test sample, a capture reaction, a capture reaction product, an amplification reaction, or an amplification reaction product) at concentrations of 5, 25, 100, and 250 copies/ml. By detecting the predetermined concentration of the SICs—for example, by using probes directed to the SICs—the skilled artisan can estimate the concentration of an organism of interest in a test sample. In certain embodiments, this is accomplished by correlating the frequency that a captured sequence is detected to the volume of the sample from which the nucleic acids were obtained. Thus, an organism count per unit volume (e.g., copies/mL for liquid samples such as blood or urine) can be estimated for each organism detected.

In particular embodiments, the concentration of SICs and probes directed to the SICs are adjusted empirically so that sequences of SICs detected in a capture reaction product and/or amplification reaction product make up about 2, 4, 6, 8, 10, 12, 14, 16, 18, 20, 25, or 30% of sequences in the mixture. In particular embodiments, SICs make up 10-20% of sequence reads. In certain embodiments, the number of SICs sequence reads in a sequencing reaction is quantitatively evaluated to ensure that sample processing occurs within pre-defined parameters. In particular embodiments, the pre-defined parameters include one or more of the following: reproducibility within two standard deviations relative to all samples sequenced during a particular run, empirically determined criteria for reliable sequencing data (e.g., base calling reliability, error scores, percentage composition of total sequencing reads for each probe per target organism), no greater than about 15% deviation of GC or AU-rich SICs within a sequencing run. In embodiments in which patient samples are barcoded to allow pooling for multiplex sequencing, the SICs DNA in a sample will also comprise the same barcode(s) corresponding to unique samples, e.g., particular patient samples.

In more particular embodiments, SICs may comprise a region of interest as defined above, where the region of interest is modified to further comprise a sequence heterologous to the region of interest. In more particular embodiments, the sequence heterologous to the region of interest in the SICs is at least 1, 2, 3, 4, 5, 6, 7, 8, 9, 10, 15, 20, 25, 30, 40 contiguous bases, or more. By using SICs comprising a modified region of interest, a single probe can be used both to detect an organism of interest within a sample, as well as the SICs, which provides internal controls for quantification and validation. Thus, SICs sequences and a region of interest from an organism of interest detected in a test sample can be differentiated by detecting the sequence heterologous to the region of interest, e.g., by sequencing or sequence-specific quantitative PCR.

### 2.2 Samples

In some embodiments, the mixtures of the invention contain sample nucleic acids. The nucleic acids may be obtained from any test sample, such as a biological sample. The nucleic acids obtained from the test sample may be of varying degrees of purity, such as at least 1, 2, 3, 4, 5, 10, 20, 30, 40, 50, 60, 70, 80, 85, 90, 95, 96, 97, 98, 99% of organic matter by weight. In particular embodiments, the sample nucleic acids are extracted from a test sample. In some embodiments, the sample nucleic acids may be further processed, for example, to allow detection of methylation state. For an overview detecting genome-wide methylation sites, see Deng (2009) (describing MIP capture of CpG islands and bisulfate sequencing to map methylation sites).

Test samples may be from any source and include samples of foodstuffs (safety testing, tagging, and tracking), agricultural samples (e.g., soil samples, for pathogen detection and/or detecting GM crops), drug lots (e.g., for lot release assays, both of small molecule and biologics, including blood supplies), water samples (including analysis of biodiversity of a water supply, safety testing (e.g., biodefense) of agricultural, commercial, government, hospital, industrial, laboratory, military, residential, or veterinary water supplies, as well as safety testing for swimming or bathing), swabs or extracts of any surface, air quality monitoring, or biological samples, such as patient samples.

Patients can include humans or animals, such as livestock, domestic, and wild animals. In some embodiments, animals are avian, bovine, canine, equine, feline, ovine, pisces/fish, porcine, primate, rodent, or ungulate. Patients may be at any stage of development, including adult, youth, fetal, or embryo. In particular embodiments, the patient is a mammal, and in more particular embodiments, a human.

Biological samples from a subject or patient may include whole cells, tissues, or organs, or biopsies comprising tissues originating from any of the three primordial germ layers—ectoderm, mesoderm or endoderm. Exemplary cell or tissue sources include skin, heart, skeletal muscle, smooth muscle, kidney, liver, lungs, bone, pancreas, central nervous tissue, peripheral nervous tissue, circulatory tissue, lymphoid tissue, intestine, spleen, thyroid, connective tissue, or gonad. Test samples may be obtained and immediately assayed or, alternatively processed by mixing, chemical treatment, fixation/preservation, freezing, or culturing. Biological samples from a subject also include blood, pleural fluid, milk, colostrums, lymph, serum, plasma, urine, cerebrospinal fluid, synovial fluid, saliva, semen, tears, and feces. Other samples include swabs, washes, lavages, discharges, or aspirates (such as, nasal, oral, nasopharyngeal, oropharyngeal, esophagal, gastric, rectal, or vaginal, swabs, washes, ravages, discharges, or aspirates), and combinations thereof, including combinations with any of the preceding biopsy materials.

### 2.3 Panels

In certain embodiments, mixtures of the invention comprise probes designed to detect a panel of organisms, such as common pathogens for a particular affliction (e.g., respiratory, blood, or urinary tract infections) or sample type (e.g., biopsies, water, foodstuff, or agricultural). “Panel” refers to a mixture provided by the invention comprising a plurality of probes directed to one or more pathogens associated with a particular affliction or sample type. In certain embodiments, the mixtures of the invention contain multiple panels. Panels comprising probes directed to particular pathogens can be produced using only routine skill by following the teachings of the present application. In some embodiments, panels provided by the invention are directed to a plurality of pathogens, such as those described in U.S. Patent Application Publication No. 2010/0098680 (particularly paragraph 160, which is incorporated herein by reference). In particular embodiments, a panel contains at least one probe directed to each of at least 1, 2, 3, 4, 5, 10, 15, 20, 25, 30, 35, 40, or 50 of the pathogens described in paragraph 160 of U.S. Patent Application Publication No. 2010/0098680.

In some embodiments, the panel is a cerebral spinal fluid (CSF) panel and comprises probes directed to Neisseria meningitides (for example, genome accession nos. NC_{—}008767, NC_{—}010120, NC_{—}003116, NC_{—}003112, NC_{—}013016, or NC_{—}004758; in particular embodiments, comprising a probe directed to the ctrA gene), HHV6 (human herpesvirus 6; e.g., genome accession nos. NC_{—}001664 or NC_{—}000898; in particular embodiments, comprising a probe directed to the major capsid protein gene), JCV (JC polyomavirus, e.g., genome accession no. NC_{—}001699.1; in particular embodiments, comprising a probe directed to the large T antigen gene), BKV (BK polyomavirus, e.g., genome accession no. NC_{—}001538; in particular embodiments, comprising a probe directed to the regulatory region), HSV1 (human herpesvirus 1, e.g., genome accession nos. NC_{—}001806 or X14112; in particular embodiments, comprising a probe directed to the gD gene (positions 138333-141048 in X14112)), HSV2 (human herpesvirus 2, e.g., genome accession nos. NC_{—}001798 or Z86099; in particular embodiments, comprising a probe directed to the gG gene (positions 137878-139977 in Z86099)), Streptococcus pneumoniae (e.g., genome accession nos. NC_{—}012469, NC_{—}012468, NC_{—}012467, NC_{—}008533, NC_{—}012466, NC_{—}010380, or NC_{—}011072; in particular embodiments, comprising a probe directed to the ply gene), Haemophilus influenza (e.g., genome accession nos. NC_{—}007146, NC_{—}000907, NC_{—}009566, NZ_AAZE00000000, NZ_AAZJ00000000, NC_{—}009567, or DQ115375; in particular embodiments, comprising a probe directed to the bexA gene). In particular embodiments a panel provided by the invention comprises one or more probes to each of 1, 2, 3, 4, 5, 6, 7, or all 8 of these organisms and, in more particular embodiments, the exemplary genes for the organisms.

In some embodiments, the panel is a meningitis panel that comprises one or more probes directed to one or more of group B streptococci, Escherichia coli, Listeria monocytogenes, Neisseria meningitides, Streptococcus pneumoniae (serotypes 6, 9, 14, 18 and 23), Haemophilus influenzae type B, staphylococci, pseudomonas, Mycobacterium tuberculosis, Treponema pallidum, Borrelia burgdorferi, Cryptococcus neoformans, Naegleria fowleri, enteroviruses, herpes simplex virus type 1 and 2, varicella zoster virus, mumps virus, HIV, LCMV, Angiostrongylus cantonensis, Gnathostoma spinigerum, Tuberculosis, syphilis, cryptococcosis, and coccidioidomycosis. In particular embodiments the panel comprises probes directed to 1, 2, 3, 4, 5, 6, 7, 8, 9, 10, 15, 20, 25, 30, or 31 of these organisms.

In some embodiments, the panel is a urinary tract infection (UTI) panel that comprises probes directed to S. saprophyticus (ATCC 15305) (e.g., genome accession nos. AP008934 or AP008935; in particular embodiments, comprising a probe directed to the gyrB gene), Enterococcus faecalis (MMH594) (e.g., genome accession no. AF034779; in particular embodiments, comprising a probe directed to the esp gene; see, e.g.,), E. coli (CFT073) (e.g., genome accession no. NC_{—}004431.1; in particular embodiments, comprising a probe directed to the fimH gene), E. coli. (IAI39) (e.g., genome accession no. NC_{—}011750.1; in particular embodiments, comprising a probe directed to the papG gene), E. coli (CFT073) (e.g., genome accession no. NC_{—}004431.1; in particular embodiments, comprising a probe directed to the papX gene), Ureaplasma urealyticum (serovar 10 str. ATCC 33699) (e.g., genome accession no. UUR10_{—}0078; in particular embodiments, comprising a probe directed to the hly gene), Ureaplasma parvum (serovar 3 str. ATCC 27815) (e.g., genome accession no. CP000942; in particular embodiments, comprising a probe directed to the hly gene), Enterococcus faecium (CV133) (e.g., genome accession no. AF544400; in particular embodiments, comprising a probe directed to the hyl(efm) gene), and Enterococcus faecium (e.g., genome accession no. AF034779; in particular embodiments, comprising a probe directed to the esp gene). In particular embodiments a mixture of nucleic acid probes provided by the invention comprises one or more probes to each of 1, 2, 3, 4, 5, 6, 7, 8, or all 9 of these organisms and, in more particular embodiments, the exemplary genes for the organisms.

In some embodiments, the panel is an alternate UTI panel comprising one or more primers to one or more organisms including Escherichia coli, Staphylococcus saprophyticus, Proteus spp., Klebsiella spp., Enterococcus spp., Candida albicans, Ureaplasma, and Mycoplasma spp. In particular embodiments a mixture of nucleic acid probes provided by the invention comprises one or more probes to each of 1, 2, 3, 4, 5, 6, 7, or all 8 of these organisms.

In still another embodiment, a UTI panel comprises one or more probes directed to E. coli. In more particular embodiments, the panel further comprises one or more probes directed to other Enterobacteriaceae, such as Klebsiella spp., Serratia spp., Citrobacter spp., and Enterobacter spp., non-fermenters such as Pseudomonas aeruginosa, and gram-positive cocci, including coagulase negative staphylococci and Enterococcus spp. In still more particular embodiments, the panel further comprises one or more probes directed to candida, such as Candida albicans. In particular embodiments a mixture of nucleic acid probes provided by the invention comprises one or more probes to each of 1, 2, 3, 4, 5, 6, 7, 8, 9, 10, or 11 of these organisms.

In some embodiments, the panel is a UTI panel comprising one or more probes directed to E. coli, Chlamydia, Mycoplasma, Staphylococcus saprophyticus, and Staphylococcus epidermidis. In particular embodiments a mixture of nucleic acid probes provided by the invention comprises one or more probes to each of 1, 2, 3, 4, or 5 of these organisms.

In certain embodiments, the panel is a respiratory panel that comprises one or more probes directed to Staphylococcus aureus, Pseudomonas aeruginosa, Klebsiella pneumoniae, Haemophilus influenza, Branhamella (Moraxella) catarrhalis, Streptococcus pyogenes (Group A), Corynebacterium diphtheriae, SARS-CoV, Bordatella pertussis, Influenza virus (types A, B, C), Rhinovirus, Coronavirus, Enterovirus, Adenovirus, Respiratory syncytial virus (RSV), Parainfluenza virus, Mumps virus, Legionella pneumophila, Pseudomonas aeruginosa, Burkholderia cepacia, Mycoplasma pneumoniae, Mycobacterium tuberculosis, Chlamydia pneumoniae, Mycobacterium aviumintracellulare complex (MAC), Candida albicans, Coccidioides immitis, Histoplasma capsulatum, Blastomyces dermatitidis, Cryptococcus neoformans, and Aspergillus fumigates. In particular embodiments a panel provided by the invention comprises one or more probes to each of 1, 2, 3, 4, 5, 6, 7, 8, 9, 10, 15, 20, 25, 30 or 33 of these organisms.

In some embodiments, the panel is a respiratory panel that contains one or more probes directed to one or more pathogens including influenza A (including subtypes H1, H3, H5 and H7), influenza B, parainfluenza (type 2), respiratory syncytial virus, and adenovirus.

In particular embodiments, the panel is a respiratory panel that contains one or more probes directed to one or more pathogens including Streptococcus pneumoniae, Mycoplasma pneumoniae, Haemophilus influenzae, Chlamydophila pneumoniae, and Legionella species, Legionella pneumophila, SARS virus, H1N1, H5N1, Gram-negative rods, Moraxella catarrhalis, Staphylococcus aureus, Tuberculosis, and respiratory syncytial virus (RSV). In particular embodiments a panel provided by the invention comprises one or more probes to each of 1, 2, 3, 4, 5, 6, 7, 8, 9, 10, 11, 12, 13, or 14 of these organisms.

In some embodiments, the panel is a blood panel comprising one or more probes directed to one or more of Diphtheria, Epstein-Barr virus (EBV), Chagas, HIV, West Nile Virus, Malaria, Syphilis, Dengue Fever, Babesia, Xenotropic Murine Leukemia Virus-related Virus (XMRV), Hepatitis B, Hepatitis C, Viral Hemorrhagic Fever (Includes Ebola and Marburg viruses). In particular embodiments a panel provided by the invention comprises one or more probes to each of 1, 2, 3, 4, 5, 6, 7, 8, 9, 10, 12, 13, or 14 of these organisms. In more particular embodiments, the blood panel comprises one or more probes to each of HIV, Hepatitis B, Hepatitis C, and Trypanosoma cruzi (Chagas). In further embodiments, the blood panel comprises one or more probes directed to each of HIV, Hepatitis B, Hepatitis C, and Trypanosoma cruzi (Chagas) pathogens, and Human host genomic sequences such as HLA, Kir, ABO and Rhesus blood marker loci.

In some embodiments, the panel is a blood panel that contains one or more probes directed to one or more pathogens including those disclosed in paragraphs 26 and 27 of U.S. Patent Application Publication No. 2009/0291854, which are incorporated herein by reference. In particular embodiments, a panel provided by the invention comprises one or more probes to each of 1, 2, 3, 4, 5, 6, 7, 8, 9, 10, 15, 20, 25, or 30 of these organisms.

In some embodiments, the panel is a sepsis panel and comprises one or more probes directed to one or more pathogens including mostly Gram-negative bacteria, like E. coli, Klebsiella, Proteus, Enterobacter species, Pseudomonas aeruginosa, Neisseria meningitidis and Bacteroides as well as common Gram-positive bacteria like Staphylococcus aureus, Streptococcus pneumoniae and other streptococci. In particular embodiments, a panel provided by the invention comprises one or more probes to each of 1, 2, 3, 4, 5, 6, 7, 8, 9, or 10 of these organisms.

In some embodiments, the panel is a water, soil, or agricultural panel and comprises one or more probes directed to, for example, G. lamblia, Cryptosporidium, Salmonella, Shigella, Campylobacter, Candida, E. coli, Yersinia, Aeromonas, or other small parasitic organisms. In certain embodiments, the panel includes one or more probes to Giardia and/or Cryptosporidium, which are common contaminants in water and/or soil. In particular embodiments a panel provided by the invention comprises one or more probes to each of 1, 2, 3, 4, 5, 6, 7, 8, 9, 10, or 11 of these organisms.

In some embodiments, the panel is a foodstuff or agricultural panel comprise one or more probes directed to one or more of Escherichia coli, Salmonella, Shigella sonnei, Campylobacter, Listeria (e.g., Listeria monocytogenes), Yersinia enterocolitica, Yersinia pseudotuberculosis, Vibrio cholera, and Clostridium (e.g., C. botulinum). In particular embodiments, a foodstuff or agricultural panel includes one or more primers directed to Escherichia coli O157:H7, enterohemorrhagic Escherichia coli (EHEC), enterotoxigenic Escherichia coli (ETEC), enteroinvasive Escherichia coli (EIEC), enteropathogenic Escherichia coli (EPEC), Salmonella, Listeria, Yersinia, Campylobacter, Clostridial species, and Staphylococcus spp. In certain embodiments, an agricultural or foodstuff panel contains one or more probes to common citrus contaminants, such as Xylella fastidiosa and Xanthomonas axonopodis. In particular embodiments, a panel provided by the invention comprises one or more probes to each of 1, 2, 3, 4, 5, 6, 7, 8, 9, 10, 15, 20, or more, of these organisms.

A fungal panel, in some embodiments, includes at least one probe directed to one or more fungi described in paragraphs 162 and 180 and Tables 1 and 2 of U.S. Patent Application Publication No. 2010/0129821, which are incorporation herein by reference. In particular embodiments, a panel provided by the invention comprises one or more probes to each of 1, 2, 3, 4, 5, 6, 7, 8, 9, 10, 15, 20, 25, or 30 of these organisms. In particular embodiments, a fungal panel comprises one or more probes directed to Aspergillus and/or Candida Albicans.

In some embodiments, panels provided by the invention comprise probes directed to plurality of pathogens as described herein, as well as probes directed to specific Human genomic sequence, such as HLA, Kir, ABO and Rhesus blood marker loci, allowing genotyping and pathogen detection in the same sample.

In some embodiments, the panel is a subject panel for genotyping a subject. In particular embodiments, the subject panel comprises probes for at least 1, 2, 3, 4, 5, 6, 7, 8, 9, 10, 20, 40, 80, 100, 200, 400, 800, 1000, 5000, or 10000 subject loci. In particular embodiments, the panel is for a mammalian subject. In more particular embodiments, the mammal is a human. In some embodiments, the panel is a prenatal or neonatal panel for detecting heritable genetic abnormalities and/or genotypes associated with increased risk for disease. In particular embodiments, the panel comprises probes for Killer cell immunoglobulin-like receptors (KIR) locus typing and to detect cytokine SNPs, e.g., one or more of the following SNPs: IL-6: C/G at −174; TNF-α: G/A at −308, G/A at −238; IL-10: G/A at −1082, C/T at −819, C/A at −592. In some embodiments the panel comprises probes to genotype HLA markers, and in particular embodiments at least one probe for each of Class I (A-H) and Class II HLA markers. In other embodiments, the panel comprises probes directed to one or more of the genes described in paragraphs 25, 57, and 58 of U.S. Patent Application Publication No. 2010/0137426, paragraphs 6 and 7 of U.S. Patent Application Publication No. 2009/0305284, paragraph 27 of U.S. Patent Application Publication No. 2010/0144836, any of the markers listed in table 1 of U.S. Patent Application Publication No. 2010/0143949, or any of the genes in paragraph 14 of U.S. Patent Application Publication No. 2010/0093558, all of which are incorporation herein by reference. In some embodiments, a panel comprises probes directed to gain of function “oncogenes” (such as ABL1, BCL1, BCL2, BCL6, CBFA2, CBL, CSF1R, ERBA, ERBB, EBRB2, ETS1, ETS1, ETV6, FGR, FOS, FYN, HCR, HRAS, JUN, KRAS, LCK, LYN, MDM2, MLL, MMTV-PyVT, MMTVneu, MYB, MYC, MYCL1, MYCN, NRAS, PIM1, PML, RET, SRC, TAL1, TCL3, and YES) and/or loss-of-function of a tumor suppressor gene (such as APC, BRCA1, BRCA2, MADH4, MCC, NF1, NF2, RB1, P53, and WTI). In some embodiments, a panel comprises probes directed to HLA, Kir and cytokine gene loci. In particular embodiments, a panel provided by the invention comprises one or more probes to each of 1, 2, 3, 4, 5, 6, 7, 8, 9, 10, 15, 20, 30, or more, of these markers.

Additional panels provided by the invention include probes directed to viral, bacterial, archaeal, protozoan, and eukaryotic organisms, as well as combinations. In particular embodiments, a panel contains at least one probe for each of about 1, 2, 3, 4, 5, 6, 7, 8, 9, 10, 15, 20, 25, 30 or 35 viruses; about 1, 2, 3, 4, 5, 6, 7, 8, 9, 10, 15, 20, 25, 30 or 35 bacteria; and about 1, 2, 3, 4, 5, 6, 7, 8, 9, 10, 15, 20, 25, 30 or 35 eukaryotes. In particular embodiments, the probes in a panel directed to eukaryotes comprise probes to at least 1, 2, 3, 4, 5, 6, 7, 8, 9, or 10 fungi. In certain embodiments, a panel may further comprise at least one probe for each of 1, 2, 3, 4, 5, 6, 7, 8, 9, or 10 archaea.

Exemplary virus taxa that can be detected with a panel of the invention include: Adenoviridae, Alloherpesviridae, Anellovirus, Arenaviridae, Arteriviridae, Ascoviridae, Asfarviridae, Astroviridae, Baculoviridae, Barnaviridae, Benyvirus, Bicaudaviridae, Birnaviridae, Bornaviridae, Bromoviridae, Bunyaviridae, Caliciviridae, Caudovirales, Caulimoviridae, Cheravirus, Chrysoviridae, Circoviridae, Closteroviridae, Comoviridae, Coronaviridae, Corticoviridae, Cystoviridae, Deltavirus, Dicistroviridae, Endornavirus, Filoviridae, Flaviviridae, Flexiviridae, Furovirus, Fuselloviridae, Geminiviridae, Globuloviridae, Hepadnaviridae, Hepeviridae, Herpesvirales, Herpesviridae, Hordeivirus, Hypoviridae, Idaeovirus, Iflavirus, Inoviridae, Iridoviridae, Leviviridae, Lipothrixviridae, Luteoviridae, Malacoherpesviridae, Marnaviridae, Microviridae, Mimiviridae, Mononegavirales, Myoviridae, Nanoviridae, Narnaviridae, Nidovirales, Nimaviridae, Nodaviridae, Ophiovirus, Orthomyxoviridae, Ourmiavirus, Papillomaviridae, Paramyxoviridae, Partitiviridae, Parvoviridae, Pecluvirus, Phycodnaviridae, Picornavirales, Picornaviridae, Plasmaviridae, Podoviridae, Polydnaviridae, Polyomaviridae, Pomovirus, Potyviridae, Poxyiridae, Reoviridae, Retroviridae, Rhabdoviridae, Roniviridae, Rudiviridae, Sadwavirus, Salterprovirus, Sequiviridae, Siphoviridae, Sobemovirus, Tectiviridae, Tenuivirus, Tetraviridae, Tobamovirus, Tobravirus, Togaviridae, Tombusviridae, Totiviridae, Tymoviridae, and Umbravirus. Non-DNA and/or single stranded viruses will readily be adapted for use in the invention by means known to the skilled artisan such as, for example, by reverse transcription. In certain embodiments, the mixtures of the invention comprise one or more probes to detect at least 1, 2, 4, 6, 8, 10, 15, 20, 30, 50, 100, 150, 200, 250, 300, or 400 types of virus.

Exemplary forms of bacteria that can be detected with a panel provided by the invention include Firmicutes (e.g., Bacillales, Lactobacillales, Clostridia), Bacteroidetes/Chlorobi, Actinbacteria, Cyanobacteria, Spirochaetales, Chlamydiae, Alpha proteobacteria (e.g., Rhizobia, Rickettsias), Beta proteobacteria (e.g., Bordetella, Neisseria, Burkholderia), Gamma proteobacteria (e.g., Pasteurella, Xanthmonas, Pseudomonas, Enterobacteria, Vibrio), as well as Epsilon and Delta proteobacteria. In certain embodiments, the mixtures of the invention comprise one or more probes to detect at least 1, 2, 4, 6, 8, 10, 15, 20, 30, 50, 100, 150, 200, 250, 300, or 400 types of bacteria.

Exemplary forms of archaea that can be detected with a panel provided by the invention include Thermococcales, Thermoplasmales, Methanosarcinales, Methanomicrobales, Methanococcales, Methanobacteriales, Methanopyrales, Halobacteriales, Archaeoglobales, Nanoarchaeota, and Crenarchaeota (e.g., Thermoproteales, Sulfolobales, and Desulfurococcales). In certain embodiments, the mixtures of the invention comprise one or more probes to detect at least 1, 2, 4, 6, 8, 10, 15, 20, 30, 50, 100, 150, 200, 250, 300, or 400 types of archaea.

Exemplary eukaryotes that can be detected with a panel provided by the invention include Nematoda, Trematoda, Diplomonadida, Apicomplexa, Entameobidae, Kinetoplastida, Dictyostellida, Stramenopiles, Fungi (e.g., Microsporidia, Basidomycota, Zygomycota, and Ascomycota (e.g., Schizosaccharomycetes, Saccharomycotina, and Pezizomycotina)). In certain embodiments, the mixtures of the invention comprise one or more probes to detect at least 1, 2, 4, 6, 8, 10, 15, 20, 30, 50, 100, 150, 200, 250, 300, or 400 types of eukaryotes.

### 3 Exemplary Methods of the Invention

### 3.1 Probe Design

The probes and mixture provided by the invention can be produced by the skilled artisan by following the examples and the general teachings of the application. The probe design process (also referred to as probe design “pipeline”) may take as input a set of genomic DNA sequences against which probes may be designed and the sets of particular strains of target organisms. The genomic DNA sequences may be entire genomes, particular genes, or genomic coordinates in one or more strains. Alternately, the pipeline may take as input a set of genomes, genes, or coordinates and will select a set of regions to target based on some criteria. The pipeline may use criteria such as regions that vary between the input genomes, genes, or coordinates of the targeted regions in the homologous probe sequence set and a larger set of known genomes.

In particular embodiments, the sequence of a target genome for the organism of interest is provided and all possible strings of consecutive nucleotides of length n (n-mers) within the target genome are enumerated (also referred to herein as “slicing” a target genome), where n is 18, 20, 22, 24, 26, 28, 30, 32, 34, 36, 38, 40, 45, 50, 55, 60, 65, 70, 80, 90, 100, 110, 120, or more. In particular embodiments, n is 18-50, 18-36, 20-32, or 22-28 nucleotides. In further particular embodiments, n is 18-26 nucleotides. In more particular embodiments, n is 22-28, e.g., 25 nucleotides. In some embodiments, the genomic segments of length n are with an offset of about between 1 and n. In particular embodiments, the offset is 1.

In certain embodiments, the enumerated n-mers are annotated to identify their genomic position. In some embodiments, the n-mers are converted to strings without genomic annotation to facilitate more rapid screening.

The pipeline may generate a first score for each n-mer according to the n-mer's suitability as a ligation-side probe homology region (a ligation-side homer) and as an extension-side probe homology region (an extension-side homer). The score for the n-mer may be based upon features such as melting temperature, general sequence composition, sequence composition at specific positions, and the n-mer's propensity to form hairpins with itself or with the backbone sequence.

The pipeline may filter n-mers to remove those of substantially the same or exactly the same sequence (i.e., a “duplicate screen”). To generate a set of candidate ligation-side homers, n-mers with the same suffix of length x, where x is the minimum n used in enumerating genomic segments of length n (as described above), are considered and the ones with the highest scores may be kept, where the scores are based on the n-mer's suitability as a ligation-side homer, as described above. To generate a set of candidate extension-side homers, n-mers with the same prefix of length x are considered and the ones with the highest scores may be kept.

In some embodiments, the scoring of n-mers may be performed as a series of screens to remove n-mers that are not suitable for use as homologous probe sequences. The screens include removing duplicate and substantially duplicate sequences, removing sequences outside of a specified Tm range (“Tₘ screen,” e.g., outside 50-72° C.), removing sequences with strings with too many repeated nucleotides (“repeat screen,” e.g., 4 or more consecutive identical nucleotides), and removing sequences likely to self-hybridize (“hairpin screen,” e.g., self-dimerize or form hairpins). These screens can be adjusted to accommodate any of the parameters described in the application for homologous probe sequences. The screens can be performed in any order, for example, by any of the embodiments in the following table:

| First screen | Second screen | Third Screen | Fourth Screen |
|---|---|---|---|
| duplicate | Tₘ screen | repeat screen | hairpin screen |
| screen | | | |
| duplicate | Tₘ screen | hairpin screen | repeat screen |
| screen | | | |
| duplicate | repeat screen | Tₘ screen | hairpin screen |
| screen | | | |
| duplicate | repeat screen | hairpin screen | Tₘ screen |
| screen | | | |
| duplicate | hairpin screen | Tₘ screen | repeat screen |
| screen | | | |
| duplicate | hairpin screen | repeat screen | Tₘ screen |
| screen | | | |
| Tₘ screen | duplicate | repeat screen | hairpin screen |
| | screen | | |
| Tₘ screen | duplicate | hairpin screen | repeat screen |
| | screen | | |
| Tₘ screen | repeat screen | duplicate | hairpin screen |
| | | screen | |
| Tₘ screen | repeat screen | hairpin screen | duplicate |
| | | | screen |
| Tₘ screen | hairpin screen | repeat screen | duplicate |
| | | | screen |
| Tₘ screen | hairpin screen | duplicate | repeat screen |
| | | screen | |
| repeat screen | hairpin screen | Tₘ screen | duplicate |
| | | | screen |
| repeat screen | hairpin screen | duplicate | Tₘ screen |
| | | screen | |
| repeat screen | Tₘ screen | hairpin screen | duplicate |
| | | | screen |
| repeat screen | Tₘ screen | duplicate | hairpin screen |
| | | screen | |
| repeat screen | duplicate | Tₘ screen | hairpin screen |
| | screen | | |
| repeat screen | duplicate | hairpin screen | Tₘ screen |
| | screen | | |
| hairpin screen | duplicate | Tₘ screen | repeat screen |
| | screen | | |
| hairpin screen | duplicate | repeat screen | Tₘ screen |
| | screen | | |
| hairpin screen | Tₘ screen | duplicate | repeat screen |
| | | screen | |
| hairpin screen | Tₘ screen | repeat screen | duplicate |
| | | | screen |
| hairpin screen | repeat screen | Tₘ screen | duplicate |
| | | | screen |
| hairpin screen | repeat screen | duplicate | Tₘ screen |
| | | screen | |

Candidate homers (or a subset thereof where the subset may be chosen based on scores generated as described above) may be aligned against a set of genomes from various strains of a target organism and against a general database of known genomes. Each homer may be assigned a second score that takes into consideration 1) the number of strains that the homer matches, and 2) the number of single nucleotide polymorphisms (SNPs) between those strains within the expected extension region, adjacent to the homer, that is to be sequenced (i.e., the number of SNPs the homer is expected to reveal given the expected read length of the sequenced extension product).

The scored (or screened) n-mers are filtered to eliminate those that specifically hybridize to a sequence in a genome in the exclusion set of genomes, e.g., comprising the genome of the subject (in the case of a biological sample) and sequenced genomes of organisms other than the organism of interest, including viruses, bacteria, archaea, fungi, and other eukaryotes. In particular embodiments, the exclusion set of genomes includes commensal organisms, non-pathogenic organisms, and pathogenic organisms other than the target organism. In particular embodiments, a screened n-mer is eliminated if it contains less than 1, 2, 3, 4, 5, 6, 7, 8, 9, or 10 mismatches in a window of 18, 19, 20, 21, 22, 23, 24, 25, 26, 27, 28, 29; 30, 35, 40, or 45 nucleotides to any sequence in the exclusion set. In particular embodiments, a screened n-mer is removed if it contains at least 19 or 20 matches in a window of at least 22 nucleotides (e.g., 25 nucleotides). The candidate n-mers can be screened against the exclusion set by any means known in the art for sequence comparison. In particular embodiments, candidate n-mers are screened by MegaBLAST against the exclusion set. In some embodiments, the screened n-mers are formatted to contain genome annotations (such as their position in the genome of the target organism), in other embodiments, they are further screened as strings without genome annotations.

In certain embodiments, screened n-mers are further screened to ensure that they specifically hybridize to a sequence in at least one additional hybridizing genome. In some embodiments, the additional hybridizing genome is an additional sequenced genome of the target organism. In particular embodiments, the additional hybridizing genome is a closely related, but distinct species, for example, belonging to the same genus or serovar. In some embodiments, the screened n-mers are screened to ensure that they specifically hybridize to the additional hybridizing genome before screening to eliminate those that specifically hybridize to the exclusion set of genomes; in other embodiments, they are screened after. In particular embodiments, screened n-mers are first screened to ensure that they specifically hybridize to the at least one additional hybridizing genome before being screened to eliminate sequences that specifically hybridize to a sequence in the exclusion set of genomes.

In some embodiments, screened n-mers are further screened to ensure that they occur in the genome of the target organism below a particular repeat threshold, such as less than 20, 19, 18, 17, 16, 15, 10, 9, 8, 7, 6, 5, 4, 3, or 2 times in the genome of the target organism. In particular embodiments, the screened n-mer occurs exactly once in the genome of the target organism.

Once the screened n-mers are further screened to ensure the desired pattern of specific hybridization (i.e., specifically hybridizing to the genome of the target organism and not specifically hybridizing to the exclusion set), the candidate ligation-side homers and extension-side homers may be assembled into candidate probes. Pairs of candidate homers may be selected to capture a predetermined region of interest, chosen by human preselection or computational methods. In other embodiments, pairs of candidate homologous probe sequences are selected to capture a region of predetermined length, e.g., at least 2, 3, 4, 5, 6, 7, 8, 9, 10, 15, 20, 30, 40, 50, 60, 80, 100, 125, 150, 200, 250, 300, 350, 400, 500, 600, 700, 800, 900, 1000, 1200, 1400, 1600, 1800, or 2000 nucleotides. In some embodiments, the homer pairs are within a maximum extension distance determined for a particular target organism strain.

A score for the candidate probes may be generated by 1) computing the number of SNPs or indels (insertions or deletions or combinations thereof), up to a selected maximum value, which are observed between each pair of strains to which the probe is expected to bind; 2) generating a sum of the values from (1) to yield the total number of SNPs or indels that the probe may reveal; and 3) multiplying the sum from (2) by an estimate of the probability that the probe will work. This product is the probe's final score. The probability that the probe works may take into account any of the following:

i) the sequence of the ligation homer;

ii) the sequence of the extension homer;

iii) the sequence of the probe's backbone;

iv) the sequence of the extension region between the two homers;

v) the two homer Tₘs;

vi) the propensity of the probe to form hairpins with itself;

vii) the sequence composition of the extension region;

viii) the sequence composition of specific parts of the extension region, n-mers, or combinations thereof; and

ix) the length of the extension region.

Alternately, the score for a probe may be generated such that the score is higher for probes that hybridize only to or preferably to a specific set of genomes or a single genome while excluding another particular set of genomes.

In some embodiments, a candidate probe's score does not include a sum of the SNPs observed between all strains of interest but instead includes a sum of the smaller of the number of SNPs observed and a particularly chosen value.

In some embodiments, probes are added to a set of final probes (an “output set”) sequentially. The probe with the highest candidate probe score, computed as described above, may be chosen first. At that point, the scores of all remaining candidate probes may be recomputed such that probes which reveal SNPs between strains that are not distinguished by previously chosen probes are scored higher and probes that reveal SNPs that distinguish between strains that are distinguished by previously chosen probes are scored lower. In some embodiments, the scores of the remaining candidate probes may be updated to reflect their propensity to cross hybridize to those probes already chosen for the output set.

Given a set of scored probes, which may be a subset of all possible probes, probes may be selected for inclusion in a final probe output set by selecting probes in order of decreasing probe score until all pairs of strains A and B, where A is in a set of strains S1, S2, S3, etc., and B is in another set of stratins, are expected to be distinguished by at least some minimum number of SNPs, indels, or both.

In some embodiments, given a set of scored probes, which may be a subset of all possible probes, probes may be selected for inclusion in a final probe output set by 1) choosing the probe with the highest score, and 2) recomputing the scores of the remaining probes by subtracting the number of SNPs or indels revealed by already chosen probes from the number revealed by probes still under consideration. In this way, a probe's score may be updated to reflect how much new information a probe provides given all previously selected probes.

Assembly of homers into probes may include insertion of backbone sequences, such as detectable moieties and primers.

In certain embodiments, mixtures of assembled probes are further screened to eliminate sequences likely to form secondary structures or specifically hybridize with other probes in the mixture.

Given a set of selected probes, the probe selection software may provide an evaluation based on the number of SNPs or indels that the probes reveal among a particular set of target organism strains. The software may display this information as an image of a 2D grid, wherein one axis is the strain or species and the other axis is a position in a particular probe's extension region and the color of that grid entry denotes the genotype of that strain/species at that position. The software may display this information as a tree where each node in the tree corresponds to a probe. The set of edges from the node may correspond to the sets of genomes which are indistinguishable according to the SNPs or indels observed by that probe and all ancestor probes in the tree.

Given a set of selected probes, the software may also provide an evaluation based on the number of strains to which each probe is expected to hybridize. The software may display this information as an image of a 2D grid wherein one axis is the genome and the other axis is a probe and the color at the intersection indicates whether the probe will hybridize to the genome, or the color may indicate the probability or likelihood of the hybridization.

In further embodiments, probes may be chosen not based on how many SNPs they reveal between sets of strains, but rather based on lists of target loci, where each loci is a single nucleotide in a single genome. The set of target loci may be derived from a base set of loci in one or more reference genomes and the complete set of target loci in all relevant genomes may be derived from the base set by aligning the reference genome to each other genome. This method is applicable, for example, to a case where drug resistance mutations have been described in a reference strain of a pathogen and probes are designed that will detect those mutations in a set of strain or isolate genomes of that pathogen.

In such methods of selecting probes based on lists of target loci, n-mers may be generated as described above. In these methods, the probability that a probe works may also be calculated as described above. However, in such methods, the final score by which probes are ranked and or chosen is typically based on the product of the probe's probability of working and the number of target loci the probe's extension region, or the expected sequencing reads of the extension region, will cover. Thus, a probe may be scored highly if it is expected to generate an informative product (meaning that the product contains target loci) against a large number of the strains of interest, and it may be scored poorly if it does not generate a product in many strains or if those products do not contain loci of interest.

In some embodiments, the final probes generated by any of the methods described herein may be modified such that the homologous probe sequences (probe arms) are no longer a perfect match to any of some set of genomes. This set of genomes may or may not be the set of genomes against which the probes were designed and may or may not be the set of genomes against which the probes were scored. In such embodiments, the parameters used to score the probe may be modified to compensate for the imperfect matches. For example, the method may have chosen probes arms with a higher than usual melting temperature and may have chosen which nucleotide or nucleotides in the probe arm to modify such that the melting temperature of the imperfect match between the probe arm and genome is within the normal range.

In particular embodiments, the methods described above take under 16, 14, 12, 10, 8, 6, or 4 days; or 72, 48, 36, 24, 12, 10, 8, 6, or 4 hours using a single core Pentium Xeon 2.5 ghz processor on a target genome of at least 10, 9, 8, 7, 6, 5, 4, 3, or 2 megabases.

Generally, probes are prepared for a particular target organism as described above. In particular embodiments, mixtures comprising probes directed to a plurality of organisms, e.g., a panel, are compiled by screening candidate probes for each target organism to be detected by the panel against each other, e.g., by pairwise comparison, to minimize or eliminate probe cross-hybridization, e.g., to eliminate probes that specifically hybridize with one or more homologous probe sequences or probe backbone sequences in the mixture.

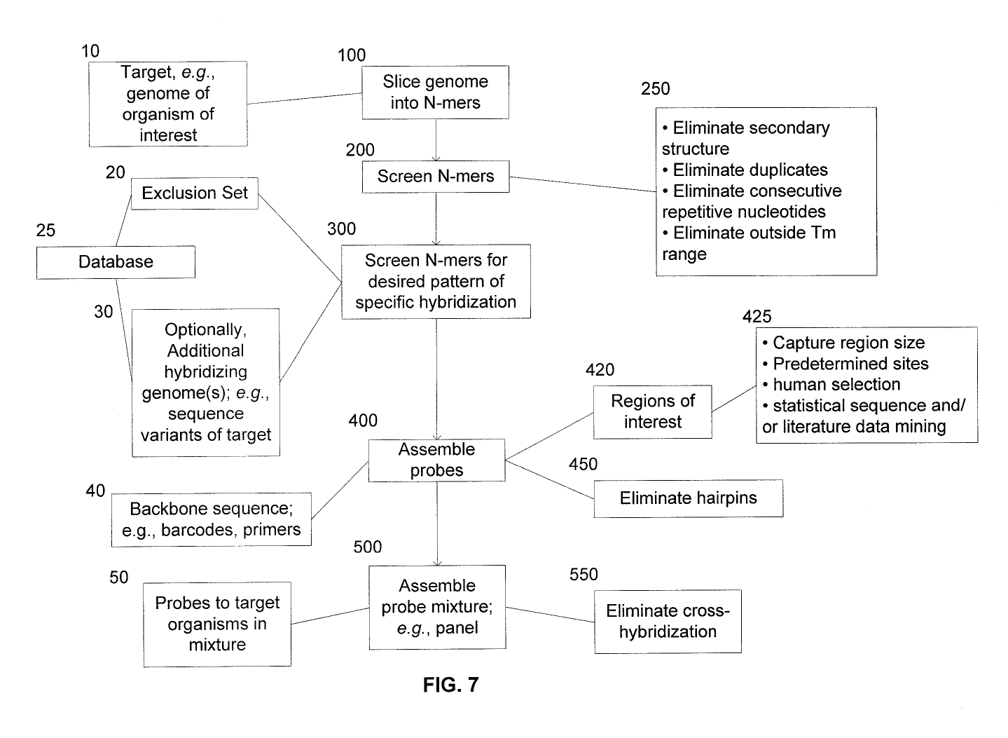

FIG. 7 is a flow chart of exemplary implementations of methods of making the probes and mixtures provided by the invention. FIG. 7, for example, depicts providing, e.g., a target genome 10, and performing a slicing 100 into a set of n-mers. The n-mers are screened by a process 200; that includes a series of screens 250 (e.g., hairpin (253), Tₘ (254), repeat (252) and duplicate (251) screens). The n-mers are then screened by a process 300 for a desired pattern of specific hybridization to an exclusion set 20 and one or more additional hybridizing genomes 30; where the exclusion set 20 and additional hybridizing genome(s) 30 are obtained from a database. For example, the process may include filtering 330 for hybridization to at least one additional hybridizing genome, filtering 340 for a repeat threshold of less than 2 (e.g., one hit per target genome), filtering 350 against a subject (e.g., human) genome, and filtering 360 against an exclusion set. The screened n-mers, if not annotated, may be annotated 370 to the target genome to determine their location in the genome. Probes are assembled in a process 400, by which pairs are filtered 420 to capture a region of interest by a filter 425, e.g., filter 425-1 to have a specified length of region of interest and to include backbone sequence 40. Probes are filtered 450 to eliminate secondary structure. A mixture of probes (e.g., a panel) is prepared by a process 500, filtered 550 to eliminate specific hybridization to other probes 50 in the mixture. Experimental validation 600 may be performed by one of skill in the art following the teaching of the application.

One of skill in the art will appreciate that although only one of each of the components identified above is depicted in the above figures, any number of any of these components may be provided. Furthermore, one of ordinary skill in the art will recognize that one or more components of any of the disclosed systems may be combined or incorporated into another component shown in the figures. One or more of the components depicted in the figures may be implemented in software on one or more computing systems. For example, they may comprise one or more applications, which may comprise one or more computer units of computer-readable instructions which, when executed by a processor, cause a computer to perform steps of a method. Computer-readable instructions may be stored on a computer-readable medium, such as a memory or disk. Such media typically provide non-transitory storage. Alternatively, one or more of the components depicted in the figures may be hardware components or combinations of hardware and software such as, for example, special purpose computers or general purpose computers. A computer or computer system may also comprise an internal or external database. The components of a computer or computer system may connect through a local bus interface.

One of skill in the art will appreciate that the above-described stages may be embodied in distinct software modules. Although the disclosed components have been described above as being separate units, one of ordinary skill in the art will recognize that functionalities provided by one or more units may be combined. As one of ordinary skill in the art will appreciate, one or more of units may be optional and may be omitted from implementations in certain embodiments.

### 3.1.1 Exemplary Algorithm for Scoring Homers and Assembled Probes

Methods of probe design, including methods as described above, may include a method for scoring homers and for scoring complete probes, wherein the score corresponds to the probability that the probe will work.

The core of the homer and probe scoring algorithm may be based on melting temperature. The logistic function is commonly used to describe the fraction of a population of nucleic acid molecules that will exist in duplex form at some temperature. If T is the experiment temperature, Tₘ is the melting temperature of the nucleic acid, and s is a parameter describing the slope of transition from duplex to dissociated, then

p(T,s)=1/(1+ê−(Tₘ−T)/s)

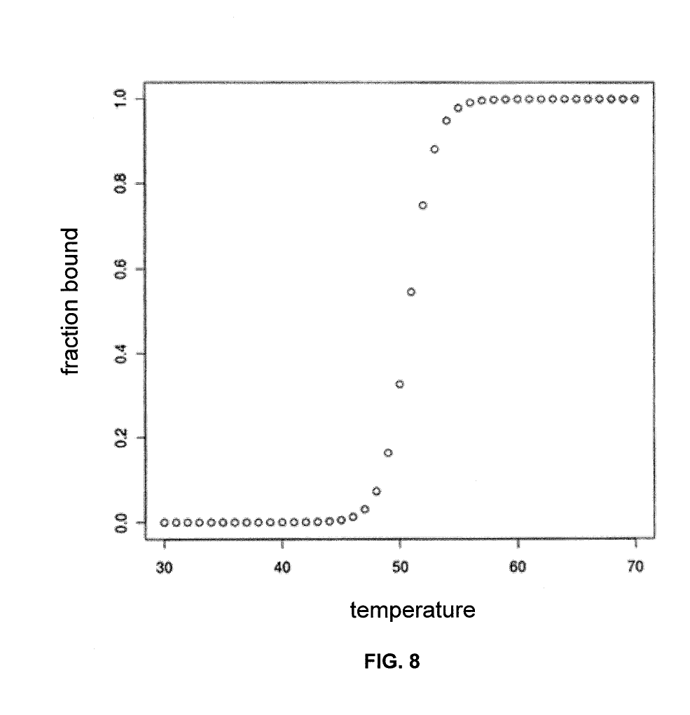

is the fraction of the population that exists in duplex form (shown as a function of Tₘ in FIG. 8). In some embodiments, for a molecular inversion probe to have a score reflecting high likelihood of successfully amplifying a target sequence, several things must happen:

1) the initiation arm of the probe must hybridize to the target nucleic acid;

2) the polymerase must initiate an extension;

3) the ligation arm of the probe must hybridize to the target nucleic acid;

4) the extension must cross the entire template sequence between the extension and ligation arms; and

5) the ligase must ligate the extension product to the ligation arm.

In some embodiments, events (1) and (3) above may be described with the logistic function based on the melting temperatures of the probe arms. Events (2) and (5) may be described in terms of the nucleotides immediately surrounding the initiation and ligation sites (e.g., each may be described by the two nucleic acids at the end of the probe arm and the two nucleic acids at the end of the extension region). Event (4) is described by the dinucleotide composition of the extension region.

Events (1) and (3) may be computed using identical formulas and parameters or may be computed differently. Tₘ may be allowed to be the melting temperature of the probe arm. The probability that the probe arm will hybridize may be described as

P_{hybOnTarget}=(p(T,s)/(p(T,s)+sumₒₜₕₑᵣ₍ₚ_{_{—}}_{other(T,s))}))*p(T,s)

where sumₒₜₕₑᵣ₍ₚ_{_{—}}_{other(T,s))} is the sum of the logistic function over the melting temperatures of the unintended or off-target matches of the probe arm to the genome. Thus, the model may describe the probability that the probe arm hybridizes as the ratio of hybridization to the intended site to the hybridization over all sites, multiplied by the probability that the probe arm hybridizes if it is available at the correct site.

The melting temperature for each match (the on-target match and some number of off-target, i.e., imperfect, matches) of the probe arm to the genome may be computed using a standard melting temperature calculator that may take into account mismatches between the probe arm and the off-target binding site, the concentration of the probe nucleic acid in the hybridization mixture, and the concentration of various ions in the hybridization mixture (e.g., Na⁺, Mg⁺⁺, K⁺, Tris).

The model may be further extended such that the sum of off-target matches includes both off-target matches, determined by inexact alignments of the probe arm sequence to the genome sequence, and a generic set of off-target matches predicted by the probe arm's Tₘ. For example, the sum of a set of predicted off-target matches may be generated, such that, at each value of t (a melting temperature of a probe arm) from 30° C. to Tₘ-k (where k=10° C.), the number of predicted off-target matches is equal to

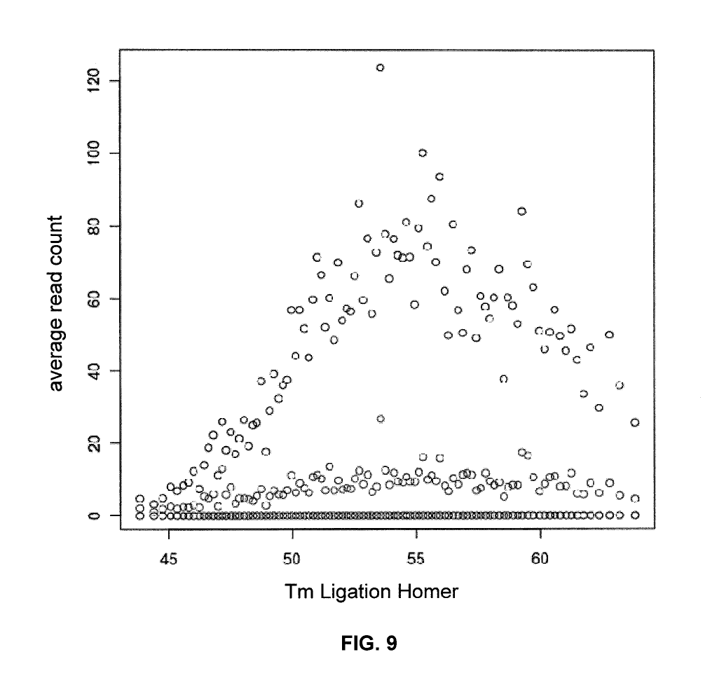

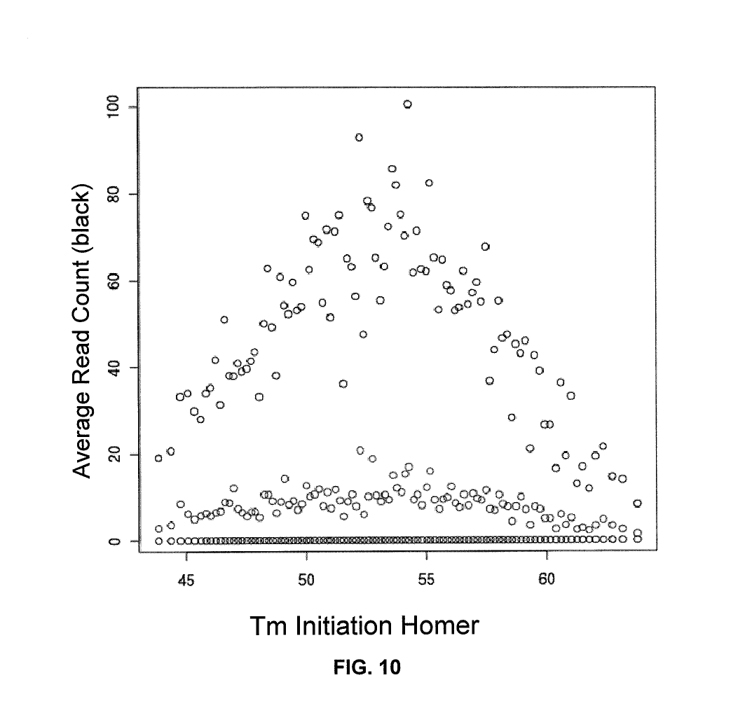

â(Tₘ−t)

where a is constant having a value of 1.4. At each value of t, the number of off-target matches or imperfect matches of the probe arm to a genome or a set of genomes is predicted according to the above formula. It is estimated that the number of off-target matches increases exponentially as t decreases. That is, the number of off-target matches may increase exponentially as the difference in melting temperature between the on-target match and the off-target match (or class of matches) increases. This may be the expected behavior as matches between the probe arm and off-target sites in the genome become shorter. Accordingly, the melting temperature may decrease and the number of such matches may become larger. The effect of melting temperature on the probe's efficiency, as determined by read count at particular melting temperatures, is shown for each of the ligation and extension probe arms (homers) in FIGS. 9 and 10, respectively (“Initiation Homer” in FIG. 10 refers to the extension probe arm; the upper arc of circles in both figures indicates the mean sequence read count for a bin of Tₘs centered around that value; the middle arc of circles in both figures [i.e., not the flat line of circles at bottom] indicates the sample standard deviation).

Event (4), the probability of a successful extension, may be described as the product of extension probabilities across the dinucleotide sequences in the extension region. Each dinucleotide may be assigned a probability that the polymerase successfully incorporates it and the probability of the polymerase crossing the extension region may be the product of these probabilities across the extension region.

Public datasets of MIP (Molecular Inversion Probe) product sequencing reads may be used to learn the parameters of the model described above, including, for example, “Multiplex amplification of large sets of human exons” by Porreca et al. Nat. Methods. November; 4(11):931-6 (2007); and “Targeted bisulfite sequencing reveals changes in DNA methylation associated with nuclear reprogramming by Deng et al., Nat. Biotechnol. 27(4):353-60 (2009).

### 3.2 Probe Capture and Detection

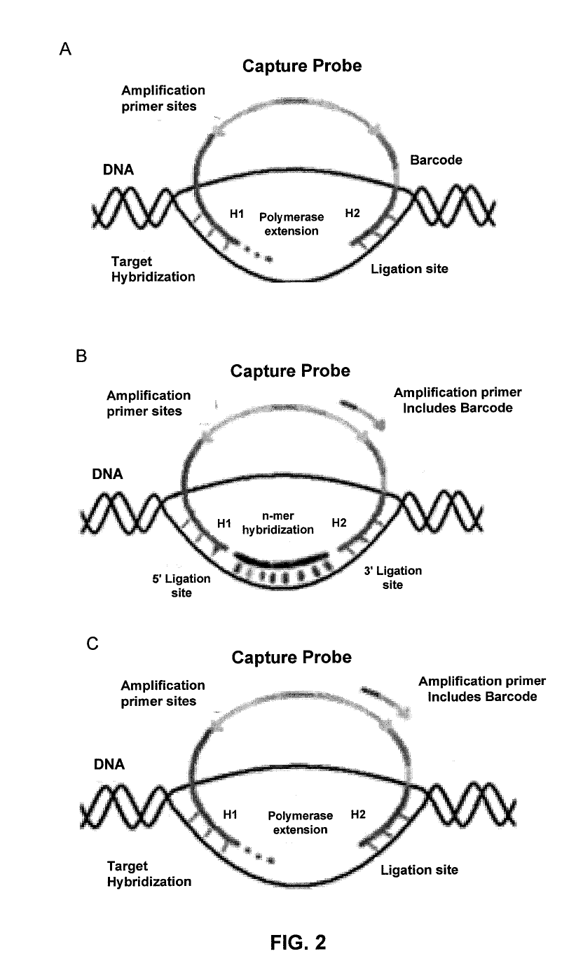

The invention provides methods of detecting the presence of one or more organisms of interest in a test sample. In certain embodiments, the methods comprise the step of contacting a mixture comprising probes described above with any of the test samples described above in a capture reaction, as defined above. In particular embodiments, a mixture comprising probes is contacted with nucleic acids extracted from a test sample, along with a polymerase enzyme and nucleotide triphosphates (NTPs), and capturing at least one region of interest by polymerase-dependent extension of at least one homologous probe sequence in the mixture. In particular embodiments, the polymerase-dependent extension of a homologous probe sequence is followed by a ligation of the end of the extended (i.e., by the polymerase) homologous probe sequence to the end of the other homologous probe sequence to produce a circularized probe containing a region of interest from the genome of an organism of interest. In some embodiments, the ligation reaction occurs while the target arm is hybridized to the target. In other embodiments, the target arm is dissociated from the target and ligated in solution under reaction conditions favoring self-ligation over trans-ligation to other probe molecules, for example a dilute ligation solution. For illustrations, see FIG. 2(A) or FIG. 2(C).

FIG. 2(C) illustrates one particular embodiment of a method provided by the invention. Briefly, hybridization of a probe to the target sequences in the organism of interest is followed by polymerase mediated, target-sequence directed addition of nucleotides to the 3′ homologous probe sequence, terminating due to obstruction at the 5′ homologous probe sequence of the probe. A ligation reaction joins the terminal 3′ nucleotide to the 5′ nucleotide of arm H2.

The sample is treated with endonuclease to digest single stranded DNA. Primers complementary to the probe backbone amplify the MIP into dsDNA for sequencing. For multiplexing of sample reaction products or amplification reaction products, amplification primers at this stage will contain sample specific nucleotide barcode sequences, e.g., they are adaptamer primers. A unique primer:barcode molecule sequence therefore identifies each test sample. For example, a panel of 100 probes is contacted with 50 individual test samples. The homologous probe sequences detected in a sequence read identifies an organism of interest, e.g., a particular pathogen or strain. Each test sample amplification reaction is done with 1 unique probe set. Each barcode within the amplification primer can be used to act as an identifier to patient, e.g., contains a barcode. Therefore 50 pairs of amplification primers (one for each amplification reaction product) and one panel of 100 probes (e.g., for 100 organisms of interest) are required for a 50 sample multiplex assay.

FIG. 2(A) illustrates an alternative embodiment. In some embodiments, each test sample is contacted with a unique set of probes, e.g., a panel. Amplification reaction products for each test sample are pooled. The homologous probe sequences and capture sequence identify both the target organism and test sample, since each test sample is contacted with a unique probe set. In some embodiments, conventional primer pairs (i.e., comprising homologous probe sequences) further comprising probe recognition sequence, are contacted with sample nucleic acids to amplify a region of interest using low cycle numbers (<10) to reduce amplification artifacts. Next, probes directed to the probe recognition sequence of the conventional primer pair amplifications products are applied. Polymerase extension and ligation captures the homologous probe sequences of the conventional primer pair and the intervening region of interest. Unique barcoded probe sequences allow for sample (e.g., patient) multiplexing. Sequence reads will comprise homologous probe sequences (identifying an organism of interest) and barcodes (associated with a sample, e.g., patient). In the example of a 100 probe panel and 50 test samples, each organism of interest has a pair of homologous probe sequences, which identify the organism of interest, e.g., a pathogen. Each test sample will be contacted with a unique probe set. Each barcode within the probe backbone can be used to act as a sample identifier. Therefore, in this illustrative embodiment, 50 sets of probes with 100 probes in each are used.

Polymerases for use in the methods provided by the invention include Taq polymerase (Lawyer et al., J. Biol. Chem., 264:6427-6437 (1989); Genbank accession:P19821), including the 5′→3′ nuclease deficient “Stoffel” fragment described in Lawyer et al., PCR Meth. Appl., 2:275-287 (1993)), PHUSION™ high fidelity recombinant polymerase (NEB), and Pyrococcus furiosus (Pfu) polymerase (see, e.g., U.S. Pat. No. 5,545,552), as well as polymerases comprising a helix-hairpin-helix domain, such as TopoTaq and PfuC2 (Pavlov et al., PNAS, 99:13510-15 (2002)). In more particular embodiments, the polymerase is 5′→3′ nuclease deficient, such as the Stoffel fragment of Taq polymerase, which further lacks 3′→5′ proofreading activity. Polymerases lacking 5′→3′ exonuclease activity may be generated by means known in the art, for example, based on methods of screening or rational design. For example, polymerase variants can be designed based on sequence alignments of one or more polymerases to the Stoffel fragment of Taq and/or by “threading” a sequence through a solved polymerase structure (e.g., MMDB IDs 56530, 81884 and 81885).

In certain embodiments, a polymerase for use in the methods of the invention is a non-displacing polymerase, such as Pfu, T4 DNA polymerase, or T7 DNA polymerase. In other embodiments, a polymerase for use in the methods provided by the invention is a polymerase suitable for isothermal amplification and caputure and/or amplification reactions are performed isothermally, e.g., by controlling metal ion concentration and/or using particular polymerases and/or additional enzymes, such as helicases or nicking enzymes (such as primer generation RCA and EXPAR). See, e.g., U.S. Pat. No. 6,566,103, Murakami et al., Nucl. Acid. Res., 37(3)e19 (2009), Tan et al., Biochemistry, 47:9987-99 (2008), Vincent et al., EMBO Rep., 5(8):795-800 (2004). Polymerases foruse in isothermal amplification include, for example, Bst, Bsu and phi29 DNA polymerases, and E. coli DNA polymerase I.

In other embodiments, a mixture of probes is contacted with nucleic acids extracted from a test sample, a ligase enzyme, and a pool of n-mer oligonucleotides in a capture reaction, as defined above. For an illustration, see FIG. 2(B). In particular embodiments, the n-mer oligonucleotides are at least 2, 3, 4, 5, 6, 7, 8, 9, 10, 12, 14, 16, 18, 20, 22, 24 or 25 nucleotides long. In more particular embodiments, they are random hexamers. In other embodiments, they are polynucleotides the length of the region of interest between the first and second target sequences that hybridize to the homologous probe sequence. In some embodiments, the n-mer oligonucleotide contains 1, 2, 3, 4, 5, 6, 7, 8, 9, or 10 locked nucleic acids (LNAs) or 10, 20, 30, 40, 50, 60, 70, 80, 90, or 100% LNAs.

The ligase enzyme ligates the n-mer oligonucleotides with the probes provided by the invention to produce a circularized probe containing a region of interest from the organism of interest. Primers complementary to the probe backbone amplify the probe into dsDNA for sequencing. In some embodiments, e.g., for multiplexing, amplification primers are adaptamer primers and contain sample-identifying barcode sequences. A unique barcode sequence therefore identifies each sample in a multiplex. Each pathogen is identified by the unique combination of homologous probe sequences and ligated n-mer in a sequence read. In more particular embodiments, the n-mer oligonucleotide is a 7-mer comprising one or more (e.g., 1, 2, 3, 4, 5, 6, or 7) locked nucleic acids and the homologous probe sequences are 10 or 12 bases, and specifically hybridize to target sequences separated by a region of interest of 7 bases.

Ligases for use in the methods of the invention include T4, T7, and thermostable ligases, such a Taq ligase (as disclosed in Takahashi et al., J. Biol. Chem., 259:10041-47 (1984), and international publication WO 91/17239), and AMPLIGASE™.

In certain other embodiments, mixtures comprising pairs of conventional PCR primers (conventional primer pairs) provided by the invention are contacted with sample nucleic acids to amplify a region of interest between two target regions in the organism of interest. In certain embodiments, a limited number of amplification steps are performed. In particular embodiments, fewer than 25, 20, 15, 10, 9, 8, 7, 6, 5, 4, 3, or 2 cycles of amplification are performed. In particular embodiments, the mixture of conventional primer pairs are contacted with nucleic acids extracted from a test sample, a polymerase, and nucleotide triphosphates to amplify the region of interest. An illustration of this methodology is shown in FIG. 3. Multiple combinations of conventional primer pairs may be used to multiplex reactions within the same sample tube, or separately for pooling. In some embodiments, primers binding to universal probe recognition sequence (e.g., a barcode) in the conventional primer pairs introduce nucleotide barcodes, and recognition sites for next-generation DNA sequencing technology primers.

As part of the present invention, conventional primer pairs can be used in a variety of additional methods. For example, in some embodiments, conventional primer pairs may be contacted with a sample nucleic acid suspected of containing at least one target nucleic acid. In particular embodiments, PCR may be used to amplify the region of interest directly from a sample nucleic acid. In other embodiments, the conventional primer pairs may be used to amplify capture reaction products, e.g., one or more circularized probes. In other embodiments a sample nucleic acid suspected of containing a region of interest is amplified using a conventional primer pair and then contacted with a probe provided by the invention for circularizing capture. In some embodiments, conventional primer pairs are contacted with a sample nucleic acid and modified nucleotides, such as biotinylated nucleotides. In some embodiments using modified nucleotides, such as biotinylated nucleotides, the resulting capture or amplification reaction products can then be isolated by affinity capture, for example, with steptavidin substrates, for subsequent processing, e.g., circularizing capture with the probes provided by the invention. In further embodiments, a single conventional primer may be used for linear amplification of a region of interest in a sample nucleic acid in, and then contacted with a probe provided by the invention for circularizing capture. In other embodiments, a single conventional primer containing a 5′ biotin moiety may be used to amplify a target sequence and then be enriched from the sample using streptavidin capture for sequencing by, for example, direct sequencing using either specific conventional primer pairs provided by the invention, or by random hexamer priming, or may be used for circularizing capture using probes provided by the invention

In certain embodiments, methods that comprise a capture reaction further comprise the step of contacting the capture reaction product with one or more exonucleases to remove linear nucleic acids. In particular embodiments, the exonuclease includes at least one of exo I, exo III, exo VII, and exo V. In more particular combinations the exonuclease is up to a 100:1, 50:1, 25:1, 10:1, 5:1, 2:1, 1:1, 1:2, 1:5, 1:10, 1:25, 1:50, or 1:100 (unit to unit) mixture of exonuclease I and exonuclease III.

In certain embodiments, the methods of the invention further comprise the step of amplifying capture reaction products in an amplification reaction. Numerous methods of amplifying nucleic acids are known in the art and include the polymerase chain reaction (see, e.g., U.S. Pat. Nos. 4,683,195 and 4,683,202 and McPherson and Moller, PCR (the baSICs), Taylor & Francis; 2 edition (Mar. 30, 2006)), OLA (oligonucleotide ligation amplification) (see, e.g., U.S. Pat. Nos. 5,185,243, 5,679,524, and 5,573,907), rolling-circle amplification (“RCA,” described in Baner et al., Nuc. Acids Res., 26:5073-78 (1998); Barany, PNAS, 88:189-93 (1991); and Lizardi et al., Nat. Genet. 19:225-32 (1998)), and strand displacement amplification (SDA; described in U.S. Pat. Nos. 5,455,166 and 5,130,238). In particular embodiments, the amplification is linear amplification such as, RCA. In more particular embodiments, capture reaction products (e.g., circularized probes) are used as templates in a RCA to generate long, linear repeating ssDNA products. In some embodiments, the RCA reaction may comprise contacting a sample with modified nucleotides, such as biotinylated nucleotides, LNA nucleotides or artificial base pairs such as IsodC or IsodG, or abasic furans (such as dSpacer), to facilitate affinity enrichment and purification. In certain embodiments, the amplification reaction products comprising linear repeating ssDNA can be contacted with a conventional primer provided by the invention to produce short extensions of double stranded DNA with a length 2, 3, 4, 5, 6, 7, 10, 15, 20, 30, 40, 50, 75, 100, 500 nucleotides. In certain embodiments, the length of extension may be controlled by time of extension step at the optimum temperature of elongation for this polymerase, e.g., 5, 10, 15, 20, 40, 60 seconds, at temperatures including 37, 42, 45, 68, 72, 74° C. In other embodiments, the length of extension is controlled by mixing of nucleotide analogues that prevented further elongation into the reaction, such as dideoxyCytosine, or nucleotides with a 3′ modification such as biotin, or a carbon spacer terminated with an amino group. In additional particular embodiments, a primer is contacted with a linear repeating ssDNA RCA amplification reaction product and extended by a polymerase for a single cycle of PCR, to generate a short single stranded DNA containing the complementary sequence to the repeating unit of the RCA product. In more particular embodiments, the primer contacted with a linear repeating ssDNA RCA amplification reaction product produces a dsDNA region comprising a restriction enzyme cleavage site. Accordingly, in certain embodiments, when the primer hybridizes to the linear repeating ssDNA RCA amplification reaction product to form a double-stranded DNA region, the amplification reaction product is contacted with the restriction enzyme to produce shorter fragments.

In particular embodiments, the amplification reaction uses adaptamer primers. In some embodiments, the amplification reaction uses sample-specific primers, that is, primers that hybridize to sequences present in the probe that identify the sample. In particular embodiments, a low number of amplification cycles are used to avoid amplification artifacts, e.g., fewer than 25, 20, 15, 10, 9, 8, 7, 6, or 5 cycles.

In certain embodiments, the methods provided by the invention may comprise the step of contacting sample nucleic acids, capture reaction products or amplification reaction products with a secondary-capture oligonucleotide capture probe which comprises a moiety designed to be captured, such as a biotin molecule, and a nucleic acid sequence, which is able to hybridize to the sample nucleic acids, capture reaction products, or amplification reaction products. Such an oligonucleotide, such as a biotinylated oligonucleotide, may be used to enrich their target nucleic acids using affinity purification. In some embodiments, a biotinylated oligonucleotide may specifically hybridize to a captured sequence (i.e., it is complementary to a region of interest), a homologous probe sequence, or a backbone sequence, such as a barcode sequence. In certain embodiments, a biotinylated probe may be extended on sample nucleic acids, capture reaction products or amplification reaction prodcts using thermophilic or mesophilic polymerases. In more particular embodiments, the method comprises contacting a capture reaction product with a biotinylated oligonucleotide for enrichment of specific capture reaction products using the biotin:streptavidin interaction.

Sequences captured by the methods of the invention can be detected by any means, including, for example, array hybridization or direct sequencing. In some embodiments, captured sequences may be detected by sequencing without amplification. Numerous sequencing methods are known in the art, can be used in the method of the invention, and are reviewed in, e.g., U.S. Pat. No. 6,946,249 and Metzker, Nat. Reviews, Genetics, 11:31-46 (2010); Ansorge, Nat. Biotechnol., 25(4):195-203 (2009), Shendure and Ji, Nat. Biotechnol., 26(10):1135-45 (2008), Shendure et al., Nat. Rev. Genet. 5:335-44 (2004). In some embodiments, the sequencing methods rely on the specificity of either a DNA polymerase or DNA ligase and include, e.g., pyrosequencing, base extension sequencing (single base stepwise extensions), multi-base sequencing by synthesis (including, e.g., sequencing with terminally-labeled nucleotides) and wobble sequencing, which is ligation-based. Extension sequencing is disclosed in, e.g., U.S. Pat. No. 5,302,509. Exemplary embodiments of terminal-phosphate-labeled nucleotides and methods of using them are described in, e.g., U.S. Pat. No. 7,361,466; U.S. Patent Publication No. 2007/0141598, published Jun. 21, 2007; and Eid et al., Science, 323:133-138 (2009). Ligase-based sequencing methods are disclosed in, for example, U.S. Pat. No. 5,750,341, PCT publication WO 06/073504, and Shendure et al., Science, 309:1728-1732 (2005). In particular embodiments, sequencing technology used in the methods provided by the invention include Sanger sequencing, microelectrophoretic sequencing, nanopore sequencing, sequencing by hybridization (e.g., array-based sequencing), real-time observation of single molecules, and cyclic-array sequencing, including pyrosequencing (e.g., 454 SEQUENCING®, see, e.g., Margulies et al., Nature, 437: 376-380 (2005)), ILLUMINA® or SOLEXA® sequencing (see, e.g., Turcatti et al., Nucleic Acids Res., 36, e25 (2008), see also U.S. Pat. Nos. 7,598,035, 7,282,370, 7,232,656, and 7,115,400), polony sequencing (e.g., SOLiD™, see Shendure et al. 2005), and sequencing by synthesis (e.g., HELICOS®, see, e.g., Harris et al., Science, 320:106-109 (2008)).

In certain embodiments, the capture probes contain sequences that facilitate processing for sequencing by a certain sequencing technology, such as sequences that can serve as anchor sites for sequencing by synthesis, primer sites for sequencing reaction initiation, or restriction enzyme sites that allow cleavage for improved ligation of oligonucleotide adaptors for sequencing of the particular amplicon. In some embodiments, circularized capture probes are contacted by oligonucleotides which prime polymerase-mediated extension of the capture probes to generate sequences complementary to that of the circularized probe, including from at least one to one million or more concatemerized copies of the original circular probe.

The mixtures and methods provided by the invention can be readily adapted to use with any suitable detections means, including, but not limited to, those listed above. In certain embodiments using ILLUMINA® or SOLEXA®sequencing, shorter homologous probe sequences may be used in the probes provided by the invention, as well as conventional primer pairs. In more particular embodiments, the homologous probe sequences will be about 8, 9, 10, 11, 12, 13, 14, 15, 16, 17, 18, 19, or 20 bases. In more particular embodiments, the region of interest between the target sequences of a probe or conventional primer pair is about 2, 3, 4, 5, 6, 7, 8, 9, 10, 11, 12, 13, 14, 15, 20, 25, 30, 35, 40, 45, or 50 bases. In still more particular embodiments, the probes provided by the invention may be circularized by polymerase-dependent synthesis and ligation, or by ligation of n-mer oligonucleotides of about 2, 3, 4, 5, 6, 7, 8, 9, 10, 11, 12, 13, 14, 15, 20, 25, 30, 35, 40, 45, or 50 bases. In yet more particular embodiments, the region of interest is about 7 bases and homologous probe sequences are 10 or 12 bases. In further embodiments a 7-mer oligonucleotide comprising a locked nucleic acid is ligated to a probe provided by the invention, and in still more particular embodiments, the 7-mer oligonucleotide comprises at least 1, 2, 3, 4, 5, 6, or 7 locked nucleic acids (LNAs).

In other embodiments, capture or amplification reaction products may be sequenced by emulsion droplet sequencing by synthesis as disclosed in, for example, Binladen et al, PLoS One. 2(2):e197 (2007). In certain embodiments, capture products may be amplified by RCA to generate higher copy numbers of capture product within a single DNA molecule in order to facilitate emulsion of captured DNA for emulsion PCR and sequencing by synthesis. See, e.g., Drmanac et al, Science 327(5961):78-81 (2010).

In particular embodiments, capture reaction products and/or amplification reaction products containing different samples are combined before detection. In particular embodiments, capture and/or amplification reaction products are combinatorially pooled before detection, e.g., an M×N array of individual capture reaction products and/or amplification reaction products are pooled by row and column, and the pools are detected. Results from row and column pools can then be deconvolved to provide results for individual samples. Higher dimensional arrays and pools may be used analogously. In other embodiments, capture reaction products and/or amplification reaction products contain identifying barcode sequences. In particular embodiments, amplification primers contain sample-specific barcode sequences. Accordingly, the sample source of sequences contained in pools of capture reaction products and/or amplification reaction products are identified by their barcode sequences.

The methods provided by the invention may also include directly detecting a particular nucleic acid in a capture reaction product or amplification reaction product, such as a particular target amplicon or set of amplicons. Accordingly, in some embodiments, the mixtures of the invention comprise specialized probe sets including TAQMAN™, which uses a hydrolyzable probe containing detectable reporter and quencher moieties, which are released by a DNA polymerase with 5′→3′ exonuclease activity (U.S. Pat. No. 5,538,848); molecular beacon, which uses a hairpin probe with reporter and quenching moieties at opposite termini (U.S. Pat. No. 5,925,517); fluorescence resonance energy transfer (FRET) primers, which use a pair of adjacent primers with fluorescent donor and acceptor moieties, respectively (U.S. Pat. No. 6,174,670); and LIGHTUP™, a single short probe which fluoresces only when bound to the target (U.S. Pat. No. 6,329,144). Similarly, SCORPION™ (U.S. Pat. No. 6,326,145) and SIMPLEPROBES™ (U.S. Pat. No. 6,635,427) use single reporter/dye probes. Amplicon-detecting probes are designed according to the particular detection modality used, and as discussed in the above-referenced patents. In particular embodiments, a quantitative, real-time PCR assay to detect a particular capture reaction product or amplification reaction product may be performed on the ILLUMINA® ECO Real-time PCR System™.

In particular embodiments, the methods of the invention comprise using sample internal calibration nucleic acid (SICs) to estimate the concentration of an organism of interest in a test sample. This is done by calibrating the frequency of a sequence from an organism of interest to the known concentration of the SICs to provide an estimated concentration of the organism of interest in the test sample. In more particular embodiments, the estimated concentration of an organism of interest is compared to a database of reference concentrations of organisms of interest associated with a disease state and/or likely clinical diagnoses.

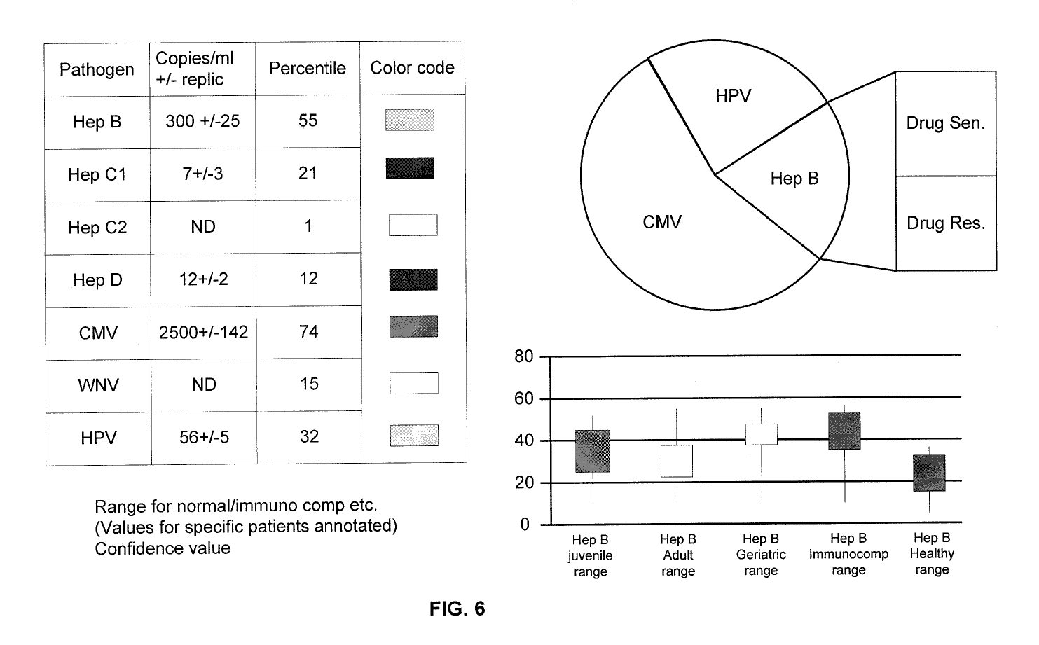

In some embodiments, the methods of the invention further comprise steps of formatting results to inform physician decision making. “Results” refers to the outcome of detecting a target organism and includes, e.g., binary (e.g., +/−) detection as well as estimates of concentration, and may be based on, inter alia the result of sequencing a capture reaction product or amplification reaction product. In particular embodiments, the formatting comprises presenting an estimate of the concentration of an organism in a test sample, optionally including statistical confidence intervals. In more particular embodiments, the formatting further comprises color coding of the results. In certain embodiments, the formatting includes recommendations for therapeutic intervention, including, for example, hospitalization, probiotic treatment, antibiotic treatments, and chemotherapy. In some embodiments, the formatting comprises one or more of the following: references to peer-reviewed medical literature and database statistics of empirically defined sample results. An exemplary format of results is shown in FIG. 6.

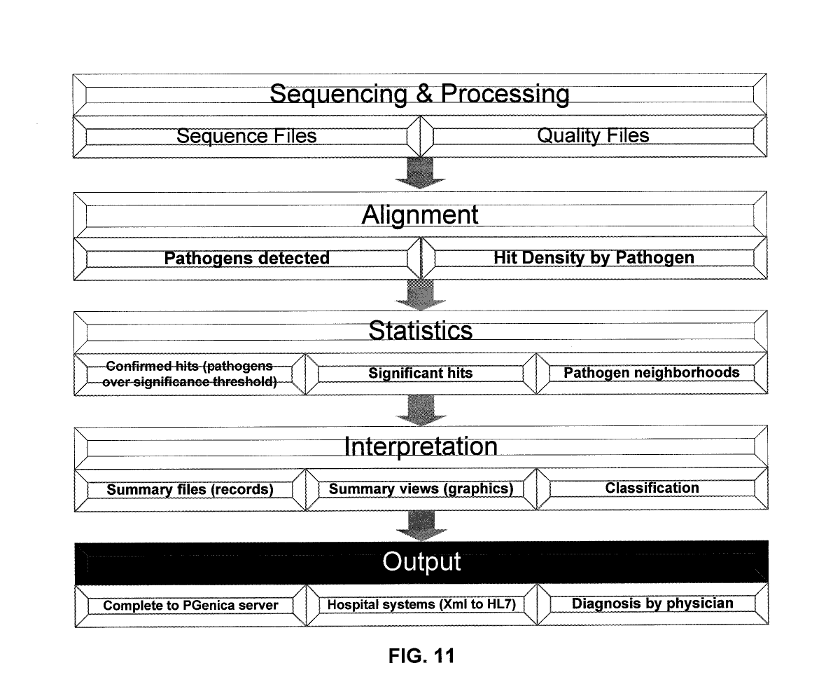

FIG. 11 is a flow chart of an exemplary embodiment of a method for, inter alia, processing, analyzing, and outputting of sequencing results.

### 3.3 Sequence Analysis

Conversion of raw sequence data may occur in three stages, namely (1) the processing of raw instrument data and conversion into aligned sequencing reads, (2) statistical interpretation of read data and (3) providing output and storage in archives.

Processing of raw data from raw instrument readout to sequence information that is associated with a location in a pathogen genome, may involve at least the two following steps:
1. Integratating sequence readout (“reads”) and associated quality score files either before or during alignment. Sequencing platform create quality scores to capture errors and identify decay of sequence with read length.
2. Aligning/mapping the reads to pathogen genomes

In some embodiments, statistical analysis and interpretation then proceed to account for all statistically significant hits against all genomes and optionally sub-classify hits by regions of interest, such as resistance loci or unique identifiers of a pathogen.

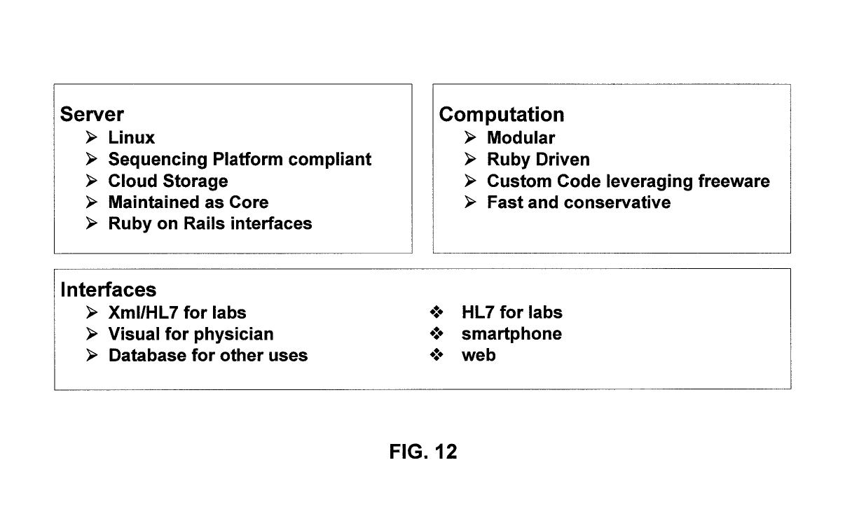

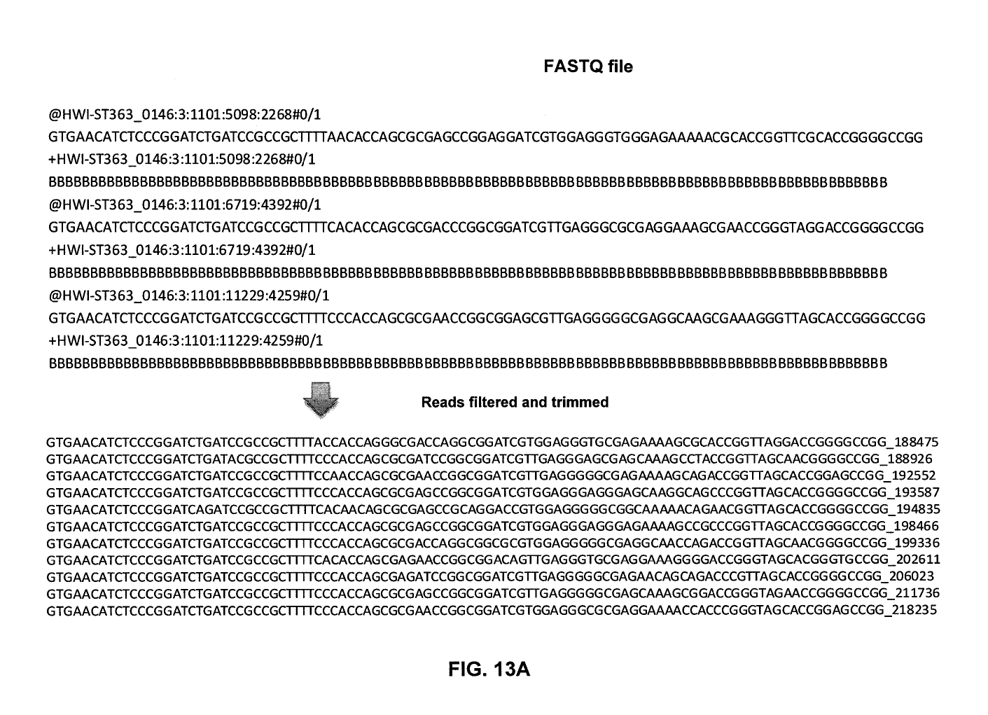

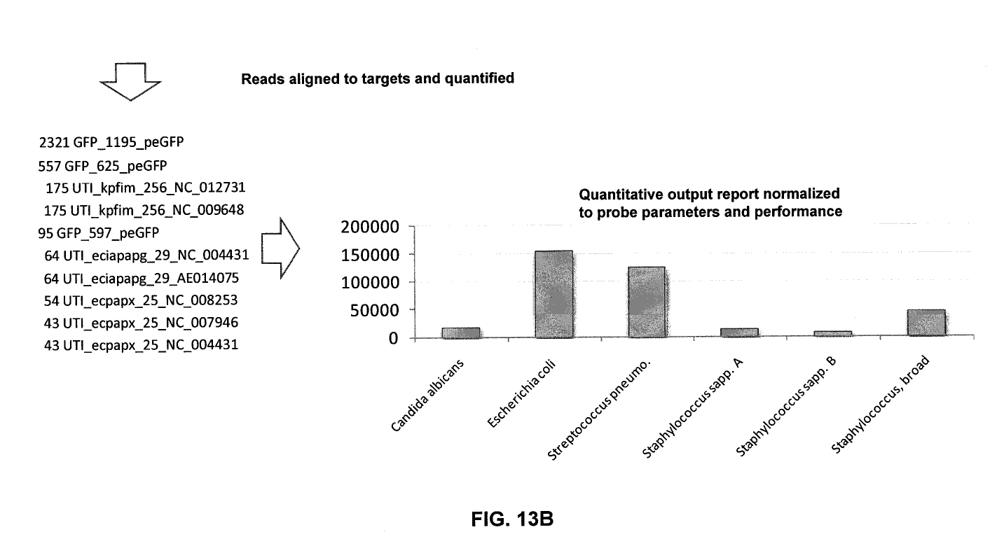

An exemplary workflow depicting processing of raw FASTQ data from a sequencing machine and quantification against reference genomes to produce quantitative analysis of organisms present within the sample is shown in FIG. 12.

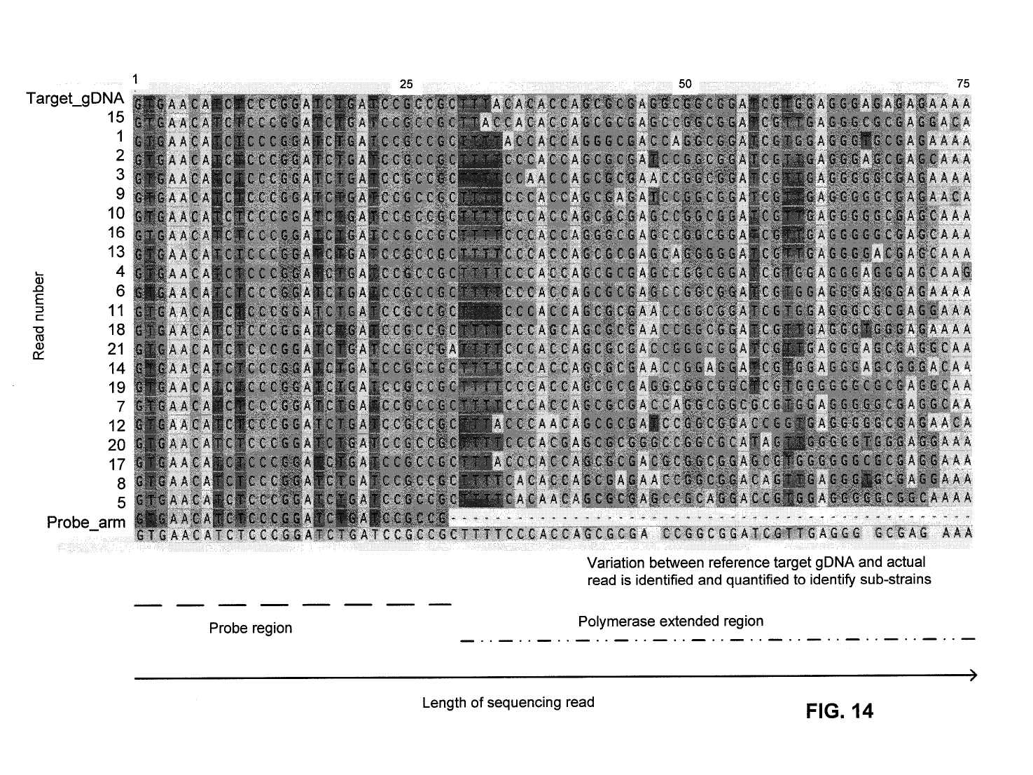

An exemplary alignment of sequences obtained from next generation sequencing reads is shown in FIG. 14. As shown here, sequencing reads may align to target genomic DNA with near-perfect matching through probe arm region. The alignment in the polymerase-extended region may reveal sequence variation through this region, which allows assignment of these amplicon sequences to different strains.

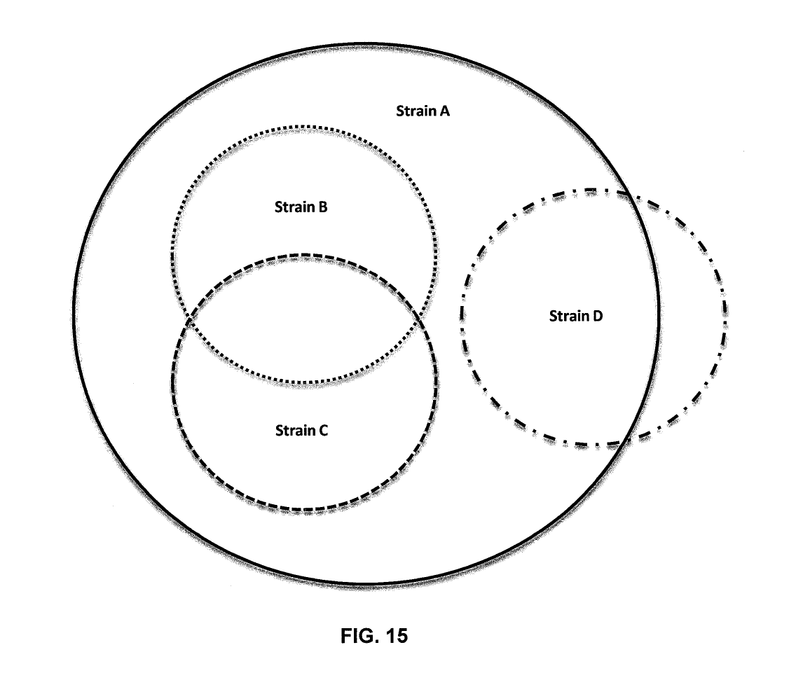

A schematic illustration of the use of sequence read alignment against a database of reference strains to identify strains in a sample is shown in FIG. 15. Some reads may map to regions common between one or more strains. In this schematic illustration, most reads align to strains A, B, C and D and are common. In contrast, other reads may be unique to specific strains (e.g., the subset of reads aligning only to strain D). In some embodiments, quantitative models are used to predict the distribution of common reads and unique reads in order to provide a quantitative estimate of the proportion of each unique pathogen present in the sample.

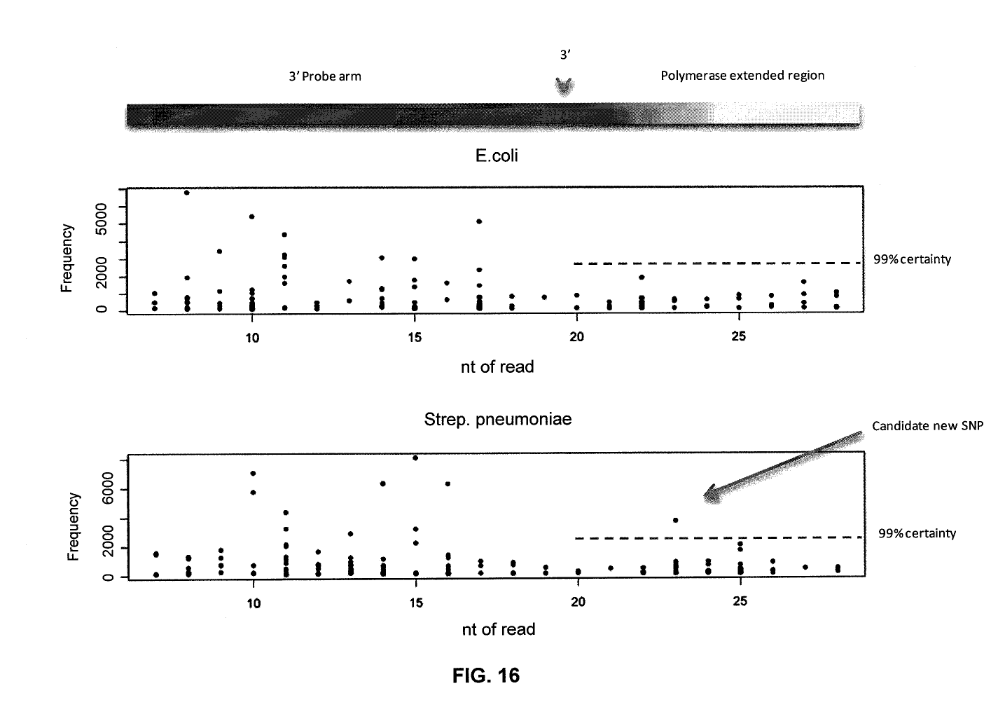

In some embodiments, accurate polymorphism modeling and detection by next generation sequencing is performed as diagramed in FIG. 16. A 3′ probe arm, polymerase extension site (arrow), and part of the polymerase-extended region are indicated at the top. The plots below indicate mismatches observed between the expected target sequence and the sequence read at each nucleotide along the sequence read. Modeling of the frequency of mismatches across the polymerase-extended region may allow accurate identification of polymorphisms that are not a result of background sequencing errors and noise.

Statistical analysis generally includes simple summary statistics, such as hit density for all pathogens, where hit density is the number of hits in a window of sequence divided by the number of high-quality reads. It can be recorded by sequence coordinates in the pathogen sequence or by a combination of a “region of interest” ID and the distance from its center. In addition, classification methodologies may be used to provide accurate assignment of samples to pathogens. The toolbox available involves maximum likelihood and Bayesian approaches, linear discriminant based methodologies and neural network approaches. This approach may employ any one or combinations of such approaches. Known methods with a proven track record in similar or related problems are hidden Markov models (HMM), Parzen Windows, multivariate regression (including LOESS regression), and support vector machines (SVMs). In some embodiments, disclosed methods employ one or more of these approaches evaluated against reference data sets in order to achieve maximum specificity and senstivity. Final analysis may depend on running many samples on a system of the invention and also on a “gold standard” reference. From this one can then examine the properties of these data, the assays and implement fixed analysis algorithms. These algorithms are not truly fixed, but instead adapt themselves to incoming data. This prior analysis is run several times over the life cycle of a system of the invention. Statistical interpretation as implemented above is dependent on prior analysis on powerful computational services. Initial analysis generates algorithmic recipes for analysis and interpretation which can then be deployed into a system of the invention.

Accordingly, in some embodiments, the goal of sequencing and subsequent analysis following a capture reaction using a set of probes is to determine the set of organisms or strains whose DNA is present in a sample. In some embodiments, a further goal is to determine the relative quantities of those organisms or strains in the sample.

Methods of analysis may rely on a model for the probability of errors in sequencing reads and a model for mutations arising between related strains of an organism. The simplest version of these models may treat all errors or changes as having equal probability, where that probability may be derived from data or chosen based on a researcher's best guess. In some embodiments, more advanced models may learn the probabilities of different types of errors from sequencing datasets of known template material using the same machine, sample preparation, and analysis software. Other advanced models may learn the probabilities of mutations based on sets of known strains from public databases of genes or genomes, private databases of genes or genomes, or from unassembled or partially assembled collections of sequencing reads.

Based on a database of known genomes and the set of probes used in the reaction, the set of expected read sequences may computed. Each expected read sequence may be derived from one probe and one genome, thus the number of expected read sequences may be the product of the number of genomes and the number of probes.

Given the set of sequencing reads (or pairs of reads) from a reaction, the reads may be aligned against the set of expected reads. Using the model for sequencing errors, the method may compute the probability that the read (or pair of reads) is derived from each expected product. The method may then compute the set of all organisms or strains that might be present in the sample as the union of the organisms/strains from all expected products to which a read aligns with greater than a selected minimum probability, for example, 0.1, 0.01, or 0.001.

In some embodiments, the methods of analysis further determine the relative proportion or abundance of each organism or strain, such that the proportions or abundances maximize the probability of actual occurance of the observed set of sequencing reads, given:
1) the probabilites of each read aligning to each expected read;
2) a prior probability of observing each organism or strain in the sample (for this type of probability, each organism or strain is equally likely);
3) a prior probability of the number of organisms or strains that will be present. In the simplest form of this type of probability, each number of organisms or strains may be equally likely. In another form, the probability of the number of organisms or strains may follow a Dirichlet distribution.

In some embodiments, the methods of analysis determine the relative proportions or abundances of organisms via a “Mixture Model.” In some embodiments, the hidden variables in the model are the proportions or abundances of the organisms or strains and the assignments of sequencing reads to expected reads (where each observed read is assigned to a single expected read). A variety of methods, including Expectation-Maximization, Gibbs Sampling, and Metropolis-Hastings, may be used to find the values of these hidden variables which maximize the probability of the data given the hidden variables and the priors on the hidden variables.

In further embodiments, the methods also incorporate unknown strains of known organisms into the Mixture Model by using the probabilities of mutations. In such embodiments, the genomes of unknown strains are generated based on observed reads that contain one or more mismatches to all known genomes. The previously unknown genome may be added to the mixture with the same probability as a known genome

Some embodiments also correct for multiple testing. Without limitation as to any one technique, the objective is to eliminate false positives and false negatives. FPR and FDR (false discovery rate) are among the most promising corrections since they are adaptable to any system. In some embodiments, thresholds are updated over time as additional cases are tested.

Exemplary embodiments categorize a sample as (1) a significant hit, (2) an inconclusive hit, (3) lack of hit or missing pathogen, or (4) poor sample quality or data error.

Output of results can occur in parallel (1) to company server, (2) to xml and HL7 formats, e.g., for deposit in hospital system, in an electronic medical record (EMR) system, or in other HL7 or xml capable storage systems, for use in existing health record frameworks, and/or (3) to physician-friendly graphical and text formats, e.g., graphs, tables, summary text and possible annotated, web formats linking to reference information. Output formats are arbitrary, e.g., simple text, spreadsheet data, binary data objects, encrypted and/or compressed files. A complete record may involve all or some of these linked to a diagnostic test via unique identifiers. They may be assembled into a coherent object or may be accessible via a search for the unique identifier.

FIG. 9 is a diagram of an exemplary embodiment of a system architecture for implementing analysis and formatting of sequencing data. This system architecture involves separation of sequencing analysis (Server), computation of statistical measures (Computation) and output or display functions (Interfaces). Many embodiments of such an architecture exist. Without limitation to any particular physical implementation, preferred embodiments include these major components in the analysis workflow and architecture.

### 3.4 Exemplary Protocols

Methods of making and using probes, capture reaction products, and amplification reaction products are known in the art and may be used in the present invention. Exemplary methods are disclosed in, e.g., Deng et al. 2009, and Li et al., Genome Res., 19(9) 1606-15 (2009).

For example, the mixtures of the present invention can be processed essentially as described in these references for capture reactions (to form capture reaction products), amplification reactions (to form amplification reaction products), and sequencing of the capture and/or amplification reaction products. The methods disclosed in these and other references are only exemplary and are in no way limiting of the present invention. For example, Deng et al. extracted Genomic DNA from frozen pellets of fibroblast, iPS or hES cells using Qiagen DNeasy columns, and bisulfite converted them with the Zymo DNA Methylation Gold Kit (Zymo Research). Bisulfate conversion may be used in the methods of the invention to study, for example, DNA methylation, but is not necessary. Deng et al. combined padlock probes (60 nM) and 200 ng of bisulfite-converted genomic DNA and mixed in 10 μl 1× Ampligase Buffer (Epicentre), denatured at 95° C. for 10 min, then hybridized at 55° C. for 18 h, after which 1 μl gap-filling mix (200 μM dNTPs, 2 U AmpliTaq Stoffel Fragment (ABI) and 0.5 units Ampligase (Epicentre) in 1× Ampligase buffer) were added to the reaction. For circularization, the reactions were incubated at 55° C. for 4 h, followed by five cycles of 95° C. for 1 min, and 55° C. for 4 h. To digest linear DNA after circularization, 2 μl exonuclease mix (containing 10 U/μl exonuclease 1 and 100 U/μl exonuclease III; USB) was added to the reaction, and the reactions were incubated at 37° C. for 2 h and then inactivated at 95° C. for 5 min.

To amplify the captured sequences, Deng et al. amplified 10-μl circularization products by PCR in 100 μl reactions with 200 nM AmpF6.2-SoL primer, 200 nM AmpR6.2-SoL primer, 0.4× SybrGreen 1 and 50 μl iProof High-Fidelity Master Mix (Bio-Rad) at 98° C. for 30 s, eight cycles of 98° C. for 10 s, 58° C. for 20 s, 72° C. for 20 s, 14 cycles of 98° C. for 10 s, 72° C. for 20 s and 72° C. for 3 min. The amplicons of the expected size range (344-394 bp) were purified with 6% PAGE (6% TBE gel; Invitrogen).

Next, Deng et al. pooled purified PCR products with the four probe sets on the same template DNA in equal molar ratio, and reamplified them in 4×100 μl reactions with 4-μl template (10-15 ng/μl), 200 μM dNTPs, 20 μM dUTP, 200 nM AmpF6.3 primer, 200 nM AmpR6.3 primer, 0.4× SybrGreen 1 and 200 μl 2× Taq Master Mix (NEB) at 94° C. for 3 min, 8 cycles of 94° C. for 45 s, 55° C. for 45 s, 72° C. for 45 s and 72° C. for 3 min. Deng et al. purified PCR amplicons with Qiaquick columns, and digested them with Mmel: ˜3.6 nmole purified PCR amplicons, 16 units of Mmel (2 U/μl; NEB), 100 μM SAM in 1×NEB Buffer 4 at 37° C. for 1 h. Deng et al. again column purified the digestions and digested with 3 U USER enzyme (1 U/μl) at 37° C. for 2 h, then with 10 units S1 nuclease (10 U/μl; Invitrogen) in 1× S1 nuclease buffer at 37° C. for 10 min. Deng et al. purified the fragmented DNA by column and end repaired the DNA at 25° C. for 45 min in 25-μl reactions containing 2.5 μl 10× buffer, 2.5 μl dNTP mix (2.5 mMeach), 2.5 μl ATP (10 mM), 1 μl end-repair enzyme mix (Epicentre), and 15 μl DNA. Approximately 100-500 ng of the end-repaired DNA was ligated with 60 μM Solexa sequencing adaptors in 30 μl of 1× QuickLigase Buffer (NEB) with 1 μl QuickLigase for 15 min at 25° C. Deng at al. size selected ligation products of 150˜175 bp in size with 6% PAGE, and amplified them by PCR in 100 μl reactions with 15 μl template, 200 nM Solexa PCR primers, 0.8× SybrGreen 1 and 50 μl iProof High-Fidelity Master Mix (Bio-Rad) at 98° C. for 30 s, 12 cycles of 98° C. for 10 s, 65° C. for 20 s, 72° C. for 20 and 72° C. for 3 min. Deng et al. purified the PCR amplicons with Qiaquick PCR purification columns, and sequenced them on an Illumina Genome Analyzer.

Li et al. used the following methods. Li et al. mixed 1× Ampligase buffer (Epicentre), 500 ng (0.25 amol) of genomic DNA (e.g., test sample DNA), and 48 ng (1.32 pmol) of probes (each probe to gDNA molar ratio=100:1; numbers change accordingly for other ratios) in a 15 μl reaction, denatured for 10 min at 95° C., ramped at 0.1° C./sec to 60° C., and then hybridized for 24 h at 60° C. They then added 2 μL of gap filling and sealing mix (5.4 μM dNTPs [100×, numbers change accordingly for 1×, 10×, 1000×, and 10,000×], two units of Taq Stoffel fragment [Applied Biosystems], and 2.5 units of Ampligase [Epicentre] in Ampligase storage buffer [Epicentre]), and incubated the reaction for 15 min, 1 h, 1 d, 2 d, or 5 d at 60° C. Li et al. also tried cycling the reaction: after 1 d at 60° C., we applied 10 cycles of 2 min at 95° C. followed by 2 h at 60° C. To remove the linear DNA, Li et al. lowered the incubation temperature to 37° C., immediately added 2 μL of Exonuclease I (20 units/μL) and 2 μL of Exonuclease III (200 units/μL) (both from USB), and incubated the reaction for 2 h at 37° C. followed by 5 min at 94° C.

Next, Li et al. amplified the circles by two 100-μL PCR reactions with 50 μL of 2× iQ SYBR Green supermix (Bio-Rad), 10 μL of circle template (from above), and 40 pmol each of forward and reverse primers (IDT). The PCR program was 3 min at 96° C.; three cycles of 30 sec at 95° C., 30 sec at 60° C., and 30 sec at 72° C.; and 10 cycles of 30 sec at 95° C., 1 min at 72° C., and 5 min at 72° C. The desired PCR products were gel purified and quantified. For each sample, Li et al. sequenced 10-20 fmol of DNA by both Illumina Genome Analyzer version 1 and updated version 2 with a custom primer.

The foregoing description has been presented for purposes of illustration. It is not exhaustive and does not limit the invention to the precise forms or embodiments disclosed. Modifications and adaptations of the invention will be apparent to those skilled in the art from consideration of the specification and practice of the disclosed embodiments. For example, the described implementations may be implemented in software, hardware, or a combination of hardware and software. Examples of hardware include computing or processing systems, such as personal computers, servers, laptops, mainframes, and micro-processors. In addition, one of ordinary skill in the art will appreciate that the records and fields shown in the figures may have additional or fewer fields, and may arrange fields differently than the figures illustrate. It is intended that the specification and examples be considered as exemplary only, with a true scope and spirit of the invention being indicated by the following claims.

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that it is incorporation herein by reference in its entirety for all purposes as well as for the proposition that is recited. Where any conflict exits between a document incorporated herein by reference and the present application, this application will control. All information associated with reference gene sequences disclosed in this application, such as GenelDs or accession numbers, including, for example, genomic loci, genomic sequences, functional annotations, allelic variants, and reference mRNA (including, e.g., exon boundaries) and protein sequences (such as conserved domain structures) are hereby incorporated herein by reference in their entirety.

### EXAMPLES

### Example 1

### Probe Generation Process

Methods are provided herein for the design of DNA oligonucleotide probes that can be used in multiplexed diagnostic assays capable of simultaneously detecting and identifying a large number of different pathogenic organisms, such as bacteria, viruses, fungi and other organisms. This is achieved by generating a pool of probes that are at once highly specific for given organisms, capable of capturing specific regions of clinical interest, and which will not cross-hybridize either with the nucleic acids of other organism or with other probes in the same pool. Candidate homology regions of DNA (or RNA) are selected, either from an entire genome (or group of genomes) or from a particular region of interest (for instance that reflect particular characteristics, such as mutations conferring drug resistance, drug sensitivity, virulence, pathogenicity, increased human transmissibility, and other features with diagnostic or clinical relevance). These homology regions can be used to identify a specific organism, strain, substrain or serovar.

In contrast to existing methods of primer design, which are limited to preselecting specific short regions of DNA (typically no more than a few thousand bases long), primers were designed according to the present methods by starting with an entire genome or group of genomes. This enables identification and validation of optimal candidate probes, from the widest possible range of nucleic acid sequences, that meet specific criteria for specificity, Tₘ, and other probe characteristics.

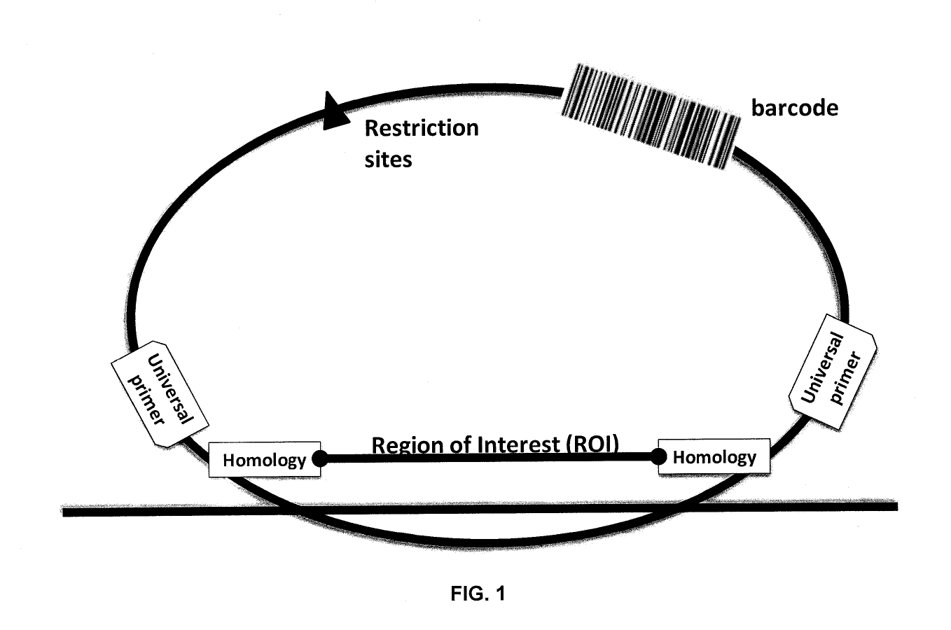

Typically, the probes provided by the present methods include two homologous probe sequences (also referred to herein as “homers”), designed to capture a region of a target organism's genome. When the homologous probe sequences of a probe hybridize to a particular target, the gap is filled and a circular product is generated, which can then be sequenced or hybridized to an array to obtain final results. A probe “backbone” connects the two homologous probe sequences and includes various linkers, DNA barcodes, amplification sites, and/or restriction sites. The assembled structure is the finished probe. A schematic of an exemplary probe provided by the invention is shown in FIG. 1.

This example describes the production of capture probes as described herein which are highly specific for two common pathogens: Streptococcus pneumonia and Salmonella enterica.

For Streptococcus pneumoniae, the target genome (gi 221230948 ref NC_{—}011900.1 Streptococcus pneumoniae ATCC 700669, complete genome) was downloaded from NCBI, along with ten additional S. pneumoniae genomes, shown below in Table 1.

TABLE 1
Additional Streptococcus pneumoniae target genomes
Target genome

gi 194172857 ref NC_003028.3 Streptococcus pneumoniae TIGR4
gi 15902044 ref NC_003098.1 Streptococcus pneumoniae R6
gi 116515308 ref NC_008533.1 Streptococcus pneumoniae D39
gi 169832377 ref NC_010380.1 Streptococcus pneumoniae
Hungary19A-6
gi 182682970 ref NC_010582.1 Streptococcus pneumoniae CGSP14
gi 194396645 ref NC_011072.1 Streptococcus pneumoniae G54
gi 225853611 ref NC_012466.1 Streptococcus pneumoniae JJA
gi 225855735 ref NC_012467.1 Streptococcus pneumoniae P1031
gi 225857809 ref NC_012468.1 Streptococcus pneumoniae 70585
gi 225860012 ref NC_012469.1 Streptococcus pneumoniae
Taiwan19F-14)

For Salmonella enterica, gi 29140543 ref NC_{—}004631.1 Salmonella enterica subsp. enterica serovar Typhi str. Ty2, complete genome, was downloaded as the initial single initial target genome. In addition, the fourteen S. enterica genomes shown in Table 2 were downloaded:

TABLE 2
Additional Salmonella enteric target genomes
Target genome

gi 161501984 ref NC_010067.1 Salmonella enterica
subsp. arizonae serovar
gi 16758993 ref NC_003198.1 Salmonella enterica
subsp. enterica serovar Typhi str. CT18
gi 161612313 ref NC_010102.1 Salmonella enterica
subsp. enterica serovar Paratyphi B str. SPB7
gi 56412276 ref NC_006511.1 Salmonella enterica
subsp. enterica serovar Paratyphi A str. ATCC 9150
gi 62178570 ref NC_006905.1 Salmonella enterica
subsp. enterica serovar Choleraesuis str. SC-B67
gi 194442203 ref NC_011080.1 Salmonella enterica
subsp. enterica serovar Newport str. SL254
gi 194733902 ref NC_011094.1 Salmonella enterica
subsp. enterica serovar Schwarzengrund str. CVM19633
gi 198241740 ref NC_011205.1 Salmonella enterica
subsp. enterica serovar Dublin str. CT_02021853
gi 197247352 ref NC_011149.1 Salmonella enterica
subsp. enterica serovar Agona str. SL483
gi 194447306 ref NC_011083.1 Salmonella enterica
subsp. enterica serovar Heidelberg str. SL476
gi 224581838 ref NC_012125.1 Salmonella enterica
subsp. enterica serovar Paratyphi C strain RKS4594
gi 207855516 ref NC_011294.1 Salmonella enterica
subsp. enterica serovar Enteritidis str. P125109
gi 205351346 ref NC_011274.1 Salmonella enterica
subsp. enterica serovar Gallinarum str. 287/91
gi 197361212 ref NC_011147.1 Salmonella enterica
subsp. enterica serovar Paratyphi A str. AKU_12601)

Next, the initial target genomes were sliced into all possible 25-base strings (25-mers) of DNA. In the example of S. pneumoniae, the initial target genome was approximately 2,253,000 bases long, and a file containing 2,221,290 strings of 25 bases each was created. For the example of S. enterica, this file contained 4,791,936 strings of 25-mers.

A series of filters was then applied to the list of 25-mer strings, which is significantly faster than with FASTA files or other formats. All duplicate sequences and any sequence with too many single repeats (5 or more) were eliminated. For S. enterica 4,295,818 candidate sequences remained after these initial filters were applied.

Next, all sequences were eliminated which are likely to form hairpins (i.e., are likely to self-hybridize) based on in silico string representations of the DNA to allow large scale rapid processing of very large candidate sets to identify probes likely to self-hybridize. The hairpin/dimerization search looks for regions within the oligonucleotide which could be self-complementary. A search criterion was established requiring that a set of N bases in the probe is matched by N complementary bases in the same probe at distance D bases away from the probe. A script created in the Ruby programming language was utilized in these implementations which first constructs a reverse complement of all possible candidate subsequences of length N derived from the probe sequence. The script then searches the probe for exact matches and reports a hairpin when a match is found and the end of the first sequence and the beginning of the second sequence are more than D bases apart. Searching and matching are performed using string manipulation functions on arrays and/or hashes of sequences that can deliver results very quickly in this setting. In this example, N is more than 3 and less than 7 and D is greater than 5.

For the candidate 25-mers from S. pneumonia, 25-mers were identified with a Tₘ of approximately 59° C., based on having a sum of guanidine and cytosine bases of exactly 13. For S. enterica, the selection for a target Tₘ was performed at a later stage, as discussed below. It was later found that performing this screen at this earlier stage substantially increased efficiency.

After applying these filters, 1,175,631 candidate sequences from Salmonella enterica remained. For the subsequent steps, string files were converted into FASTA-formatted files.

Next, NCBI's MegaBLAST Version 2.2.10 (unless otherwise indicated, any reference to BLAST [i.e., blast, blasted, BLASTed, et cetera] in the Examples refers to MegaBLAST) was used to compare all candidate 25-mers to all target genomes of the same organism listed in Tables 1 and 2 for S. pneumoniae and S. enterica, respectively. Any candidate 25-mer that did not have an exact match in all of the genomes for its target organism was discarded. For S. enterica, 42, 907 candidate 25-mers remained after this step. The number of hits for each 25-mer against each target genome was then determined, and in this example, only those that occurred exactly once in the genome were kept.

To avoid hybridization to the human genome, candidate 25-mers were BLASTed against the human genome, which was downloaded from NCBI by individual chromosome. The sequences used in these studies are shown in Table 3. Candidate 25-mers that shared 19 out of 20 consecutive bases with a sequence in the human genome were discarded. In the case of Salmonella enterica, 42,485 candidate 25-mers remained after this step.

TABLE 3
Human genomic sequences for screening of hybridizing probes
Genomic sequence

gi 89161185 ref NC_000001.9 NC_000001 Homo sapiens
chromosome 1
gi 89161199 ref NC_000002.10 NC_000002 Homo sapiens
chromosome 2
gi 89161205 ref NC_000003.10 NC_000003 Homo sapiens
chromosome 3
gi 89161207 ref NC_000004.10 NC_000004 Homo sapiens
chromosome 4
gi 51511721 ref NC_000005.8 NC_000005 Homo sapiens
chromosome 5
gi 89161210 ref NC_000006.10 NC_000006 Homo sapiens
chromosome 6
gi 89161213 ref NC_000007.12 NC_000007 Homo sapiens
chromosome 7
gi 51511724 ref NC_000008.9 NC_000008 Homo sapiens
chromosome 8
gi 89161216 ref NC_000009.10 NC_000009 Homo sapiens
chromosome 9
gi 89161187 ref NC_000010.9 NC_000010 Homo sapiens
chromosome 10
gi 51511727 ref NC_000011.8 NC_000011 Homo sapiens
chromosome 11
gi 89161190 ref NC_000012.10 NC_000012 Homo sapiens
chromosome 12
gi 51511729 ref NC_000013.9 NC_000013 Homo sapiens
chromosome 13
gi 51511730 ref NC_000014.7 NC_000014 Homo sapiens
chromosome 14
gi 51511731 ref NC_000015.8 NC_000015 Homo sapiens
chromosome 15
gi 51511732 ref NC_000016.8 NC_000016 Homo sapiens
chromosome 16
gi 51511734 ref NC_000017.9 NC_000017 Homo sapiens
chromosome 17
gi 51511735 ref NC_000018.8 NC_000018 Homo sapiens
chromosome 18
gi 42406306 ref NC_000019.8 NC_000019 Homo sapiens
chromosome 19
gi 51511747 ref NC_000020.9 NC_000020 Homo sapiens
chromosome 20
gi 51511750 ref NC_000021.7 NC_000021 Homo sapiens
chromosome 21
gi 89161203 ref NC_000022.9 NC_000022 Homo sapiens
chromosome 22
gi 89161218 ref NC_000023.9 NC_000023 Homo sapiens
chromosome X
gi 89161220 ref NC_000024.8 NC_000024 Homo sapiens
chromosome Y

After eliminating 25-mers with similarity to the human genome, the remaining 25-mers were BLASTed against an NCBI database of 25,991 microbial and 3,602 viral genomes. 25-mers that shared at least 19 of 20 consecutive bases to a sequence in any of these genomes were eliminated. After applying this filter, 2,245 candidate 25-mers for S. enterica remained.

For S. enterica, the selection for a Tₘ of approximately 59° C. (by selecting only those sequences that have a sum of guanidine and cytosine bases of exactly 13) was performed at this stage, leaving 1,116 candidate 25-mers.

The remaining candidate 25-mers for each organism were then BLASTed against their original target genome to determine their start and stop positions in the genome (i.e., their genomic coordinates). Using this information, pairs of 25-mers were selected that were separated by a fixed distance. For S. enterica, probe pairs that spanned a target length of exactly 100 bases (from the start of the first 25-mer to the end of the second 25-mer) were selected, resulting in eighteen such candidate probe pairs. In the case of S. pneumoniae, a total of 58 probes were designed for targetting sequences having lengths of 100, 200, 300, 400 and 500 bases. The 25-mers contained in the probes for S. pneumoniae are shown in Table 4, which indicates the probes' genomic location and target length.

Next, the 25-mer pairs were assembled into completed probes, using the generic linker AGATCGGAAGAGCGTCGTGTAGGGAAAGCTGAGCAAATGTTATCGAGGTC. (SEQ ID NO:7). The assembled probes for S. pneumoniae are shown in Table 5. Assembled pairs of homologous probe sequences for S. enterica are shown in Table 6, which includes the genomic location information for each pair of homologous probe sequences.

In further embodiments, before probe assembly, candidate 25-mers are BLASTed against all other candidate 25-mers and/or assembled probes in a mixture to eliminate those that would cross-hybridize with any other sequence in the mixture (e.g., homologous probe sequence, backbone, or assembled probe). In one embodiment, 25-mers that contain 19 of 20 consecutive bases contained in another probe sequence (e.g., backbone or homologous probe sequence) in the mixture are eliminated.

Once filtered, 25-mers are assembled into candidate probes, comprising two 25-mers and a backbone, which may include a variety of linkers, DNA barcodes, universal amplification primers, and other sequences as needed. Next, assembled probes may be BLASTed against all other assembled probes in the pool as an alternate or additional screen for possible cross-hybridization. Final analyses for hairpins and/or self hybridization are performed. Validated, assembled probes are then added to a database of useful probes. A flowchart of exemplary implementations in the generation process for a probe or probe mixture (e.g., a probe panel) is shown in FIG. 7.

TABLE 4
25-mer sequences for S. pneumonia-targetted probes

| | | | Target | Target | Target | | |
|---|---|---|---|---|---|---|---|
| Probe ID | H1 pos. | H2 pos. | Start | End | Length | H1 (extension arm) | H2 (ligation arm) |
| >strep.pneumo- | 645- | 720- | 645 | 744 | 100 | TATGGAGGACCAGGCCTTGGTAAGA | GCGCGTGTTAAATATATCCCTGCCG |
| 01 | 669 | 744 | | | | (SEQ ID NO: 8) | (SEQ ID NO: 9) |
| >strep.pneumo- | 673097- | 673172- | 673097 | 673196 | 100 | GGTGTTGCGCAACCTGTTTCTGTTC | GCGGCTCGTCAAATCTTTGACCTTC |
| 02 | 673121 | 673196 | | | | (SEQ ID NO: 10) | (SEQ ID NO: 11) |
| >strep.pneumo- | 707096- | 707171- | 707096 | 707195 | 100 | CAGCCTGGTTACCCAGTTCTTACTG | GGTGAGAACGAAGACAAGAACCGTC |
| 03 | 707120 | 707195 | | | | (SEQ ID NO: 12) | (SEQ ID NO: 13) |
| >strep.pneumo- | 720981- | 721056- | 720981 | 721080 | 100 | AATTCATCGGGTGACCCTGTGGAAG | ATTGTGGATCGTGTTCCAGCCTTGG |
| 04 | 721005 | 721080 | | | | (SEQ ID NO: 14) | (SEQ ID NO: 15) |
| >strep.pneumo- | 767921- | 767996- | 767921 | 768020 | 100 | AGGTGTCAATGCCATGCGTGGTGAA | CACACCTGATGTGGTACACGTGATG |
| 05 | 767945 | 768020 | | | | (SEQ ID NO: 16) | (SEQ ID NO: 17) |
| >strep.pneumo- | 777532- | 777607- | 777532 | 777631 | 100 | CGACGGGATTTATCGGTGGCTTTAC | TTGTCCAGGTGGCAGAAGATACTCG |
| 06 | 777556 | 777631 | | | | (SEQ ID NO: 18) | (SEQ ID NO: 19) |
| >strep.pneumo- | 865658- | 865733- | 865658 | 865757 | 100 | CTTCAGCGTTGTCTGTCGCCAGTAA | CAACACGACGAATCAGTTCACTGGC |
| 07 | 865682 | 865757 | | | | (SEQ ID NO: 20) | (SEQ ID NO: 21) |
| >strep.pneumo- | 963949- | 964024- | 963949 | 964048 | 100 | CCTAGTGAGATTGTCCGTGACTTGC | GAATTAGCCAAGTTTGAGCGTCCGG |
| 08 | 963973 | 964048 | | | | (SEQ ID NO: 22) | (SEQ ID NO: 23) |
| >strep.pneumo- | 1313943- | 1314018- | 1313943 | 1314042 | 100 | GCCCACCTTACCCATAGAAATGGTC | CAAGTCTAAGACATCCTGCTCCGTG |
| 09 | 1313967 | 1314042 | | | | (SEQ ID NO: 24) | (SEQ ID NO: 25) |
| >strep.pneumo- | 1348377- | 1348452- | 1348377 | 1348476 | 100 | GGCCCACATACTCATCAAGGTTGAC | ATTCAAGTGGGCTACTTCCTGTCGC |
| 10 | 1348401 | 1348476 | | | | (SEQ ID NO: 26) | (SEQ ID NO: 27) |
| >strep.pneumo- | 1421943- | 1422018- | 1421943 | 1422042 | 100 | CATCCTCGCTAGCAATTGCAGCTAG | TGGCCTGAGGATAGAAACCAATCCC |
| 11 | 1421967 | 1422042 | | | | (SEQ ID NO: 28) | (SEQ ID NO: 29) |
| >strep.pneumo- | 1471291- | 1471366- | 1471291 | 1471390 | 100 | GATTCTTCTGTCGCAGAAGCCAAGC | TTACTCTCATCCGCATTAGCCGACG |
| 12 | 1471315 | 1471390 | | | | (SEQ ID NO: 30) | (SEQ ID NO: 31) |
| >strep.pneumo- | 1528931- | 1529006- | 1528931 | 1529030 | 100 | AATGCCACACTACGGTGTTGTCCAC | CTTGGCAGAATCGGCTCAATCAAGG |
| 13 | 1528955 | 1529030 | | | | (SEQ ID NO: 32) | (SEQ ID NO: 33) |
| >strep.pneumo- | 1553284- | 1553359- | 1553284 | 1553383 | 100 | GCCGCAAAGAAGACACCAGCATCTA | ACCACAGAAAGGGCGGTTAATAGGG |
| 14 | 1553308 | 1553383 | | | | (SEQ ID NO: 34) | (SEQ ID NO: 35) |
| >strep.pneumo- | 1665069- | 1665144- | 1665069 | 1665168 | 100 | CGTGCCCTGTTGGAAAGGCAATTGA | CGATACCTTGTCCCATAGCTCCACT |
| 15 | 1665093 | 1665168 | | | | (SEQ ID NO: 36) | (SEQ ID NO: 37) |
| >strep.pneumo- | 1780734- | 1780809- | 1780734 | 1780833 | 100 | TTGACCTCAGCGATTACCTGCAAGC | GGCTGGATTTGCTCCAGCTTCATCT |
| 16 | 1780758 | 1780833 | | | | (SEQ ID NO: 38) | (SEQ ID NO: 39) |
| >strep.pneumo- | 1822203- | 1822278- | 1822203 | 1822302 | 100 | AGAGCTTCTTTCATGAGTGGAGCCC | TAACGCTCCAATTCCGCATCAGTCG |
| 17 | 1822227 | 1822302 | | | | (SEQ ID NO: 40) | (SEQ ID NO: 41) |
| >strep.pneumo- | 1832185- | 1832260- | 1832185 | 1832284 | 100 | GCCGCCCTTGAGCCTGATTTGATTA | CCAACCGTTCTCTTCCAAGCAAGCA |
| 18 | 1832209 | 1832284 | | | | (SEQ ID NO: 42) | (SEQ ID NO: 43) |
| >strep.pneumo- | 1836264- | 1836339- | 1836264 | 1836363 | 100 | CTTGGCTCAAGTCATGCTCCATCTG | CTGTCACAACGGGAACACGGGTATA |
| 19 | 1836288 | 1836363 | | | | (SEQ ID NO: 44) | (SEQ ID NO: 45) |
| >strep.pneumo- | 1888158- | 1888233- | 1888158 | 1888257 | 100 | CCGCTTCGAGCAATTGCTCAAAGAC | GGTAAGAAACAGAACCTGAAGCGCC |
| 20 | 1888182 | 1888257 | | | | (SEQ ID NO: 46) | (SEQ ID NO: 47) |
| >strep.pneumo- | 1939796- | 1939871- | 1939796 | 1939895 | 100 | ATAGCTGGACGCATGAGGTTGACTG | ACTCTTGTGACTAGAGCACCGTGAG |
| 21 | 1939820 | 1939895 | | | | (SEQ ID NO: 48) | (SEQ ID NO: 49) |
| >strep.pneumo- | 1960075- | 1960150- | 1960075 | 1960174 | 100 | GGACGGGTAAAGCGTGAGATTTGTG | TCAGCCAAACCGTTCAAGACTCCTG |
| 22 | 1960099 | 1960174 | | | | (SEQ ID NO: 50) | (SEQ ID NO: 51) |
| >strep.pneumo- | 1991584- | 1991659- | 1991584 | 1991683 | 100 | CGTGGACGAGTCAGATAGACACGAT | ACGTTCTAACCAAGCTTGACAGCCC |
| 23 | 1991608 | 1991683 | | | | (SEQ ID NO: 52) | (SEQ ID NO: 53) |
| >strep.pneumo- | 1993533- | 1993608- | 1993533 | 1993632 | 100 | CTACTTCTGCAGCCAGTTCTGGATG | CGCCACGGTCTGCAACATGTTCTTT |
| 24 | 1993557 | 1993632 | | | | (SEQ ID NO: 54) | (SEQ ID NO: 55) |
| >strep.pneumo- | 2014591- | 2014666- | 2014591 | 2014690 | 100 | CACCCGGGTCTCTCATATAAGTTGG | TCCCACGAATCTTAGCACCTGTTGC |
| 25 | 2014615 | 2014690 | | | | (SEQ ID NO: 56) | (SEQ ID NO: 57) |
| >strep.pneumo- | 2040994- | 2041069- | 2040994 | 2041093 | 100 | GCTGCGCGCTCCATTTCAAATAGAG | AGAATGGCACGTTGGAGAACGATGG |
| 26 | 2041018 | 2041093 | | | | (SEQ ID NO: 58) | (SEQ ID NO: 59) |
| >strep.pneumo- | 2051649- | 2051724- | 2051649 | 2051748 | 100 | CCTGAAGAAGGTAAGAGTCTCACCC | AAGGCAAGCCAAGTCAGTATGGCTG |
| 27 | 2051673 | 2051748 | | | | (SEQ ID NO: 60) | (SEQ ID NO: 61) |
| >strep.pneumo- | 2064289- | 2064364- | 2064289 | 2064388 | 100 | AGTCAACTGACTGGCATCTACACCG | ATTTCGGCCAAAGGGAGCCACATTG |
| 28 | 2064313 | 2064388 | | | | (SEQ ID NO: 62)_ | (SEQ ID NO: 63) |
| >strep.pneumo- | 2161108- | 2161183- | 2161108 | 2161207 | 100 | GTGCGGTTCGGAGATACGCAAGTAA | GACACTATTGAACGACGTGCTGACG |
| 29 | 2161132 | 2161207 | | | | (SEQ ID NO: 64) | (SEQ ID NO: 65) |
| >strep.pneumo- | 70613- | 70788- | 70613 | 70812 | 200 | CATCGTTGGCGTATTCGTCAGTACC | TTCCATGGCAACCAGCATAGCATCC |
| 30 | 70637 | 70812 | | | | (SEQ ID NO: 66) | (SEQ ID NO: 67) |
| >strep.pneumo- | 459298- | 459473- | 459298 | 459497 | 200 | CTGGTGCTGAGGACAAGTACAAGGA | TTTCTCAAGTTTCTTCGGCGGAGGC |
| 31 | 459322 | 459497 | | | | (SEQ ID NO: 68) | (SEQ ID NO: 69) |
| >strep.pneumo- | 891891- | 892066- | 891891 | 892090 | 200 | GATTGGTCCAATAGTGCCCGATACG | TTCCTCTTCTGCCAGTCTATGCTGG |
| 32 | 891915 | 892090 | | | | (SEQ ID NO: 70) | (SEQ ID NO: 71) |
| >strep.pneumo- | 952083- | 952258- | 952083 | 952282 | 200 | CCTTGCAGTTGGTTCGAAACCAAGG | GGCATACGGTTGGATTTCGGTTGCA |
| 33 | 952107 | 952282 | | | | (SEQ ID NO: 72) | (SEQ ID NO: 73) |
| >strep.pneumo- | 1077528- | 1077703- | 1077528 | 1077727 | 200 | GAGGTCCAAACGATTCTCAACCTGC | GCTGAACGAACATTGGCCAGACTTG |
| 34 | 1077552 | 1077727 | | | | (SEQ ID NO: 74) | (SEQ ID NO: 75) |
| >strep.pneumo- | 1079629- | 1079804- | 1079629 | 1079828 | 200 | CTTGGCCTGCTCTCTCGTTTCAAAC | AAAGGCAATGGACTCTTCCAAGCCC |
| 35 | 1079653 | 1079828 | | | | (SEQ ID NO: 76) | (SEQ ID NO: 77) |
| >strep.pneumo- | 1320102- | 1320277- | 1320102 | 1320301 | 200 | TATCGGTTGGGTACGTTCAGGTGCT | CAATTCCCTGTCTCAGCTAGATCCG |
| 36 | 1320126 | 1320301 | | | | (SEQ ID NO: 78) | (SEQ ID NO: 79) |
| >strep.pneumo- | 1377167- | 1377342- | 1377167 | 1377366 | 200 | CTCCTGAATAGCAGACAGATAGGCG | AAGACCAGAGCCGAAATTCCGTGTG |
| 37 | 1377191 | 1377366 | | | | (SEQ ID NO: 80) | (SEQ ID NO: 81) |
| >strep.pneumo- | 1543996- | 1544171- | 1543996 | 1544195 | 200 | CATCCATGAGACGAGTCATGGTGTC | AGTTTGACGGTTCTCAGGTACACGG |
| 38 | 1544020 | 1544195 | | | | (SEQ ID NO: 82) | (SEQ ID NO: 83) |
| >strep.pneumo- | 1567063- | 1567238- | 1567063 | 1567262 | 200 | TGAAGGGCTTGATTAGCCGTGAACG | TCCACTCTGGTGGTTTATCCGCATC |
| 39 | 1567087 | 1567262 | | | | (SEQ ID NO: 84) | (SEQ ID NO: 85) |
| >strep.pneumo- | 1594512- | 1594687- | 1594512 | 1594711 | 200 | CTGCCATGCCACTAGTAGCACCAAA | GCCATCTCCACGATCATTGAGGCTA |
| 40 | 1594536 | 1594711 | | | | (SEQ ID NO: 86) | (SEQ ID NO: 87) |
| >strep.pneumo- | 1837870- | 1838045- | 1837870 | 1838069 | 200 | AGTCGCTCAAACTGTTAACGCCACC | AAACGGTGATGGAGTGGTCCAGCAT |
| 41 | 1837894 | 1838069 | | | | (SEQ ID NO: 88) | (SEQ ID NO: 89) |
| >strep.pneumo- | 1904806- | 1904981- | 1904806 | 1905005 | 200 | GTGCCCACTCTATCGCTTCTTCTAG | GTCCGAACTAGCTTGCTTGTTGAGG |
| 42 | 1904830 | 1905005 | | | | (SEQ ID NO: 90) | (SEQ ID NO: 91) |
| >strep.pneumo- | 1943489- | 1943664- | 1943489 | 1943688 | 200 | TCGTACTGGGCAGGTGTCATGATGT | CAAAGGAAGCCTGTAAGCGTGTCTG |
| 43 | 1943513 | 1943688 | | | | (SEQ ID NO: 92) | (SEQ ID NO: 93) |
| >strep.pneumo- | 2061201- | 2061376- | 2061201 | 2061400 | 200 | ACCAAACCTTCAAGAAGCGGAGCCA | TAGCAGTCATAGGTGCCTCCTGGTT |
| 44 | 2061225 | 2061400 | | | | (SEQ ID NO: 94) | (SEQ ID NO: 95) |
| >strep.pneumo- | 2179622- | 2179797- | 2179622 | 2179821 | 200 | TTCCAGCGAGCTGCGTCAAATTGAC | TGATGGCTTGGATGACTTTGCGAGC |
| 45 | 2179646 | 2179821 | | | | (SEQ ID NO: 96) | (SEQ ID NO: 97) |
| >strep.pneumo- | 626697- | 626972- | 626697 | 626996 | 300 | CCACCAGATAATTGACGGGCAAAGC | GTTGAGGCAACGAAGGAGGGTACTT |
| 46 | 626721 | 626996 | | | | (SEQ ID NO: 98) | (SEQ ID NO: 99) |
| >strep.pneumo- | 1120572- | 1120847- | 1120572 | 1120871 | 300 | CAACCTGACGTCCACCTGCATAAGA | CCGTGAGTACGAATTCCTCCATCAG |
| 47 | 1120596 | 1120871 | | | | (SEQ ID NO: 100) | (SEQ ID NO: 101) |
| >strep.pneumo- | 1153293- | 1153568- | 1153293 | 1153592 | 300 | GTATCCTCTATCGTTTGGCGGAGGA | GTTCACTTGCGACTGGTCAAACACC |
| 48 | 1153317 | 1153592 | | | | (SEQ ID NO: 102) | (SEQ ID NO: 103) |
| >strep.pneumo- | 1309537- | 1309812- | 1309537 | 1309836 | 300 | TAGACCGCGACTGAGTTCGTTTGCA | CTATCCACACCACCACGCTTATGGA |
| 49 | 1309561 | 1309836 | | | | (SEQ ID NO: 104) | (SEQ ID NO: 105) |
| >strep.pneumo- | 1434430- | 1434705- | 1434430 | 1434729 | 300 | GTTCTTGCGGTTCATCTGTTCCACC | AAGTAACCACCTGCTGAGAGCAAGG |
| 50 | 1434454 | 1434729 | | | | (SEQ ID NO: 106) | (SEQ ID NO: 107) |
| >strep.pneumo- | 1437830- | 1438105- | 1437830 | 1438129 | 300 | GGAGCAGGTGCTGACACTTCTTCAT | CACCTCCGCATAGCTCTTTCCTTCT |
| 51 | 1437854 | 1438129 | | | | (SEQ ID NO: 108) | (SEQ ID NO: 109) |
| >strep.pneumo- | 1006724- | 1007099- | 1006724 | 1007123 | 400 | CGTCCCTCTTAAAGAAGCAAGCCGT | GATTTCACCACCAAACTTCCTCGGG |
| 52 | 1006748 | 1007123 | | | | (SEQ ID NO: 110) | (SEQ ID NO: 111) |
| >strep.pneumo- | 2102469- | 2102844- | 2102469 | 2102868 | 400 | TCAGCTGCATTTGGATCTGCTCCAC | TCATTCACACCTTCATCTGGCCGAG |
| 53 | 2102493 | 2102868 | | | | (SEQ ID NO: 112) | (SEQ ID NO: 113) |
| >strep.pneumo- | 347420- | 347795- | 347420 | 347819 | 400 | CTGTATCGAGTCACATGGTCCAGCA | AAGGACGAGCATATCCTCTATGCCC |
| 54 | 347444 | 347819 | | | | (SEQ ID NO: 114) | (SEQ ID NO: 115) |
| >strep.pneumo- | 162037- | 162512- | 162037 | 162536 | 500 | CCATTAGGATTCCAGGTCCCATTGC | CGCAAACTCGATAATGAGCTGGAGG |
| 55 | 162061 | 162536 | | | | (SEQ ID NO: 116) | (SEQ ID NO: 117) |
| >strep.pneumo- | 879373- | 879848- | 879373 | 879872 | 500 | GAGTACACTCCAGATGTAACGGCTC | TCGGTGGTGGAGATTCAAGCTCAAG |
| 56 | 879397 | 879872 | | | | (SEQ ID NO: 118) | (SEQ ID NO: 119) |
| >strep.pneumo- | 993493- | 993968- | 993493 | 993992 | 500 | ACCTGCAGGTTGATGAACGAGATCG | CAATCTCTTGGTCTTGGACGAGCCA |
| 57 | 993517 | 993992 | | | | (SEQ ID NO: 120) | (SEQ ID NO: 121) |
| >strep.pneumo- | 1119326- | 1119801- | 1119326 | 1119825 | 500 | CACGGAGACTCTTGACACTAGACTC | AGGGCACCAAGAAAGGCTTCAAAGG |
| 58 | 1119350 | 1119825 | | | | (SEQ ID NO: 122) | (SEQ ID NO: 123) |

TABLE 5
Assembled probe sequences for Streptococcus pneumoniae

| Probe ID | Assembled Probe |
|---|---|
| >strep.pneumo- | GCGCGTGTTAAATATATCCCTGCCGAGATCGGAAGAGCGTCGTGTAGGGAAAGCTGAGCAAATGTTATCGAGGTCTA |
| 01 | TGGAGGACCAGGCCTTGGTAAGA |
| | (SEQ ID NO: 124) |
| >strep.pneumo- | GCGGCTCGTCAAATCTTTGACCTTCAGATCGGAAGAGCGTCGTGTAGGGAAAGCTGAGCAAATGTTATCGAGGTCGG |
| 02 | TGTTGCGCAACCTGTTTCTGTTC |
| | (SEQ ID NO: 125) |
| >strep.pneumo- | GGTGAGAACGAAGACAAGAACCGTCAGATCGGAAGAGCGTCGTGTAGGGAAAGCTGAGCAAATGTTATCGAGGTCCA |
| 03 | GCCTGGTTACCCAGTTCTTACTG |
| | (SEQ ID NO: 126) |
| >strep.pneumo- | ATTGTGGATCGTGTTCCAGCCTTGGAGATCGGAAGAGCGTCGTGTAGGGAAAGCTGAGCAAATGTTATCGAGGTCAA |
| 04 | TTCATCGGGTGACCCTGTGGAAG |
| | (SEQ ID NO: 127) |
| >strep.pneumo- | CACACCTGATGTGGTACACGTGATGAGATCGGAAGAGCGTCGTGTAGGGAAAGCTGAGCAAATGTTATCGAGGTCAG |
| 05 | GTGTCAATGCCATGCGTGGTGAA |
| | (SEQ ID NO: 128) |
| >strep.pneumo- | TTGTCCAGGTGGCAGAAGATACTCGAGATCGGAAGAGCGTCGTGTAGGGAAAGCTGAGCAAATGTTATCGAGGTCCG |
| 06 | ACGGGATTTATCGGTGGCTTTAC |
| | (SEQ ID NO: 129) |
| >strep.pneumo- | CAACACGACGAATCAGTTCACTGGCAGATCGGAAGAGCGTCGTGTAGGGAAAGCTGAGCAAATGTTATCGAGGTCCT |
| 07 | TCAGCGTTGTCTGTCGCCAGTAA |
| | (SEQ ID NO: 130) |
| >strep.pneumo- | GAATTAGCCAAGTTTGAGCGTCCGGAGATCGGAAGAGCGTCGTGTAGGGAAAGCTGAGCAAATGTTATCGAGGTCCC |
| 08 | TAGTGAGATTGTCCGTGACTTGC |
| | (SEQ ID NO: 131) |
| >strep.pneumo- | CAAGTCTAAGACATCCTGCTCCGTGAGATCGGAAGAGCGTCGTGTAGGGAAAGCTGAGCAAATGTTATCGAGGTCGC |
| 09 | CCACCTTACCCATAGAAATGGTC |
| | (SEQ ID NO: 132) |
| >strep.pneumo- | ATTCAAGTGGGCTACTTCCTGTCGCAGATCGGAAGAGCGTCGTGTAGGGAAAGCTGAGCAAATGTTATCGAGGTCGG |
| 10 | CCCACATACTCATCAAGGTTGAC |
| | (SEQ ID NO: 133) |
| >strep.pneumo- | TGGCCTGAGGATAGAAACCAATCCCAGATCGGAAGAGCGTCGTGTAGGGAAAGCTGAGCAAATGTTATCGAGGTCCA |
| 11 | TCCTCGCTAGCAATTGCAGCTAG |
| | (SEQ ID NO: 134) |
| >strep.pneumo- | TTACTCTCATCCGCATTAGCCGACGAGATCGGAAGAGCGTCGTGTAGGGAAAGCTGAGCAAATGTTATCGAGGTCGA |
| 12 | TTCTTCTGTCGCAGAAGCCAAGC |
| | (SEQ ID NO: 135) |
| >strep.pneumo- | CTTGGCAGAATCGGCTCAATCAAGGAGATCGGAAGAGCGTCGTGTAGGGAAAGCTGAGCAAATGTTATCGAGGTCAA |
| 13 | TGCCACACTACGGTGTTGTCCAC |
| | (SEQ ID NO: 136) |
| >strep.pneumo- | ACCACAGAAAGGGCGGTTAATAGGGAGATCGGAAGAGCGTCGTGTAGGGAAAGCTGAGCAAATGTTATCGAGGTCGC |
| 14 | CGCAAAGAAGACACCAGCATCTA |
| | (SEQ ID NO: 137) |
| >strep.pneumo- | CGATACCTTGTCCCATAGCTCCACTAGATCGGAAGAGCGTCGTGTAGGGAAAGCTGAGCAAATGTTATCGAGGTCCG |
| 15 | TGCCCTGTTGGAAAGGCAATTGA |
| | (SEQ ID NO: 138) |
| >strep.pneumo- | GGCTGGATTTGCTCCAGCTTCATCTAGATCGGAAGAGCGTCGTGTAGGGAAAGCTGAGCAAATGTTATCGAGGTCTT |
| 16 | GACCTCAGCGATTACCTGCAAGC |
| | (SEQ ID N0: 139) |
| >strep.pneumo- | TAACGCTCCAATTCCGCATCAGTCGAGATCGGAAGAGCGTCGTGTAGGGAAAGCTGAGCAAATGTTATCGAGGTCAG |
| 17 | AGCTTCTTTCATGAGTGGAGCCC |
| | (SEQ ID NO: 140) |
| >strep.pneumo- | CCAACCGTTCTCTTCCAAGCAAGCAAGATCGGAAGAGCGTCGTGTAGGGAAAGCTGAGCAAATGTTATCGAGGTCGC |
| 18 | CGCCCTTGAGCCTGATTTGATTA |
| | (SEQ ID NO: 141) |
| >strep.pneumo- | CTGTCACAACGGGAACACGGGTATAAGATCGGAAGAGCGTCGTGTAGGGAAAGCTGAGCAAATGTTATCGAGGTCCT |
| 19 | TGGCTCAAGTCATGCTCCATCTG |
| | (SEQ ID NO: 142) |
| >strep.pneumo- | GGTAAGAAACAGAACCTGAAGCGCCAGATCGGAAGAGCGTCGTGTAGGGAAAGCTGAGCAAATGTTATCGAGGTCCC |
| 20 | GCTTCGAGCAATTGCTCAAAGAC |
| | (SEQ ID NO: 143) |
| >strep.pneumo- | ACTCTTGTGACTAGAGCACCGTGAGAGATCGGAAGAGCGTCGTGTAGGGAAAGCTGAGCAAATGTTATCGAGGTCAT |
| 21 | AGCTGGACGCATGAGGTTGACTG |
| | (SEQ ID NO: 144) |
| >strep.pneumo- | TCAGCCAAACCGTTCAAGACTCCTGAGATCGGAAGAGCGTCGTGTAGGGAAAGCTGAGCAAATGTTATCGAGGTCGG |
| 22 | ACGGGTAAAGCGTGAGATTTGTG |
| | (SEQ ID NO: 145) |
| >strep.pneumo- | ACGTTCTAACCAAGCTTGACAGCCCAGATCGGAAGAGCGTCGTGTAGGGAAAGCTGAGCAAATGTTATCGAGGTCCG |
| 23 | TGGACGAGTCAGATAGACACGAT |
| | (SEQ ID NO: 146) |
| >strep.pneumo- | CGCCACGGTCTGCAACATGTTCTITAGATCGGAAGAGCGTCGTGTAGGGAAAGCTGAGCAAATGTTATCGAGGTCCT |
| 24 | ACTTCTGCAGCCAGTTCTGGATG |
| | (SEQ ID NO: 147) |
| >strep.pneumo- | TCCCACGAATCTTAGCACCTGTTGCAGATCGGAAGAGCGTCGTGTAGGGAAAGCTGAGCAAATGTTATCGAGGTCCA |
| 25 | CCCGGGTCTCTCATATAAGTTGG |
| | (SEQ ID NO: 148) |
| >strep.pneumo- | AGAATGGCACGTTGGAGAACGATGGAGATCGGAAGAGCGTCGTGTAGGGAAAGCTGAGCAAATGTTATCGAGGTCGC |
| 26 | TGCGCGCTCCATTTCAAATAGAG |
| | (SEQ ID NO: 149) |
| >strep.pneumo- | AAGGCAAGCCAAGTCAGTATGGCTGAGATCGGAAGAGCGTCGTGTAGGGAAAGCTGAGCAAATGTTATCGAGGTCCC |
| 27 | TGAAGAAGGTAAGAGTCTCACCC |
| | (SEQ ID NO: 150) |
| >strep.pneumo- | ATTTCGGCCAAAGGGAGCCACATTGAGATCGGAAGAGCGTCGTGTAGGGAAAGCTGAGCAAATGTTATCGAGGTCAG |
| 28 | TCAACTGACTGGCATCTACACCG |
| | (SEQ ID NO: 151) |
| >strep.pneumo- | GACACTATTGAACGACGTGCTGACGAGATCGGAAGAGCGTCGTGTAGGGAAAGCTGAGCAAATGTTATCGAGGTCGT |
| 29 | GCGGTTCGGAGATACGCAAGTAA |
| | (SEQ ID NO: 152) |
| >strep.pneumo- | TTCCATGGCAACCAGCATAGCATCCAGATCGGAAGAGCGTCGTGTAGGGAAAGCTGAGCAAATGTTATCGAGGTCCA |
| 30 | TCGTTGGCGTATTCGTCAGTACC |
| | (SEQ ID NO: 153) |
| >strep.pneumo- | TTTCTCAAGTTTCTTCGGCGGAGGCAGATCGGAAGAGCGTCGTGTAGGGAAAGCTGAGCAAATGTTATCGAGGTCCT |
| 31 | GGTGCTGAGGACAAGTACAAGGA |
| | (SEQ ID NO: 154) |
| >strep.pneumo- | TTCCTCTTCTGCCAGTCTATGCTGGAGATCGGAAGAGCGTCGTGTAGGGAAAGCTGAGCAAATGTTATCGAGGTCGA |
| 32 | TTGGTCCAATAGTGCCCGATACG |
| | (SEQ ID NO: 155) |
| >strep.pneumo- | GGCATACGGITGGATITCGGTTGCAAGATCGGAAGAGCGTCGTGTAGGGAAAGCTGAGCAAATGTTATCGAGGTCCC |
| 33 | TTGCAGTTGGTTCGAAACCAAGG |
| | (SEQ ID NO: 156) |
| >strep.pneumo- | GCTGAACGAACATTGGCCAGACTTGAGATCGGAAGAGCGTCGTGTAGGGAAAGCTGAGCAAATGTTATCGAGGTCGA |
| 34 | GGTCCAAACGATTCTCAACCTGC |
| | (SEQ ID NO: 157) |
| >strep.pneumo- | AAAGGCAATGGACTCTTCCAAGCCCAGATCGGAAGAGCGTCGTGTAGGGAAAGCTGAGCAAATGTTATCGAGGTCCT |
| 35 | TGGCCTGCTCTCTCGTTTCAAAC |
| | (SEQ ID NO: 158) |
| >strep.pneumo- | CAATTCCCTGTCTCAGCTAGATCCGAGATCGGAAGAGCGTCGTGTAGGGAAAGCTGAGCAAATGTTATCGAGGTCTA |
| 36 | TCGGTIGGGTACGTTCAGGTGCT |
| | (SEQ ID NO: 159) |
| >strep.pneumo- | AAGACCAGAGCCGAAATTCCGTGTGAGATCGGAAGAGCGTCGTGTAGGGAAAGCTGAGCAAATGTTATCGAGGTCCT |
| 37 | CCTGAATAGCAGACAGATAGGCG |
| | (SEQ ID NO: 160) |
| >strep.pneumo- | AGTTTGACGGTTCTCAGGTACACGGAGATCGGAAGAGCGTCGTGTAGGGAAAGCTGAGCAAATGTTATCGAGGTCCA |
| 38 | TCCATGAGACGAGTCATGGTGTC |
| | (SEQ ID NO: 161) |
| >strep.pneumo- | TCCACTCTGGTGGTTTATCCGCATCAGATCGGAAGAGCGTCGTGTAGGGAAAGCTGAGCAAATGTTATCGAGGTCTG |
| 39 | AAGGGCTTGATTAGCCGTGAACG |
| | (SEQ ID NO: 162) |
| >strep.pneumo- | GCCATCTCCACGATCATTGAGGCTAAGATCGGAAGAGCGTCGTGTAGGGAAAGCTGAGCAAATGTTATCGAGGTCCT |
| 40 | GCCATGCCACTAGTAGCACCAAA |
| | (SEQ ID NO: 163) |
| >strep.pneumo- | AAACGGTGATGGAGTGGTCCAGCATAGATCGGAAGAGCGTCGTGTAGGGAAAGCTGAGCAAATaTTATCGAGGTCAG |
| 41 | TCGCTCAAACTGTTAACGCCACC |
| | (SEQ ID NO: 164) |
| >strep.pneumo- | GTCCGAACTAGCTTGCTTGTTGAGGAGATCGGAAGAGCGTCGTGTAGGGAAAGCTGAGCAAATGTTATCGAGGTCGT |
| 42 | GCCCACTCTATCGCTTCTTCTAG |
| | (SEQ ID NO: 165) |
| >strep.pneumo- | CAAAGGAAGCCTGTAAGCGTGTCTGAGATCGGAAGAGCGTCGTGTAGGGAAAGCTGAGCAAATGTTATCGAGGTCTC |
| 43 | GTACTGGGCAGGTGTCATGATGT |
| | (SEQ ID NO: 166) |
| >strep.pneumo- | TAGCAGTCATAGGTGCCTCCTGGTTAGATCGGAAGAGCGTCGTGTAGGGAAAGCTGAGCAAATGTTATCGAGGTCAC |
| 44 | CAAACCTTCAAGAAGCGGAGCCA |
| | (SEQ ID NO: 167) |
| >strep.pneumo- | TGATGGCTTGGATGACTTTGCGAGCAGATCGGAAGAGCGTCGTGTAGGGAAAGCTGAGCAAATGTTATCGAGGTCTT |
| 45 | CCAGCGAGCTGCGTCAAATTGAC |
| | (SEQ ID NO: 168) |
| >strep.pneumo- | GTTGAGGCAACGAAGGAGGGTACTTAGATCGGAAGAGCGTCGTGTAGGGAAAGCTGAGCAAATGTTATCGAGGTCCC |
| 46 | ACCAGATAATTGACGGGCAAAGC |
| | (SEQ ID NO: 169) |
| >strep.pneumo- | CCGTGAGTACGAATTCCTCCATCAGAGATCGGAAGAGCGTCGTGTAGGGAAAGCTGAGCAAATGTTATCGAGGTCCA |
| 47 | ACCTGACGTCCACCTGCATAAGA |
| | (SEQ ID NO: 170) |
| >strep.pneumo- | GTTCACTTGCGACTGGTCAAACACCAGATCGGAAGAGCGTCGTGTAGGGAAAGCTGAGCAAATGTTATCGAGGTCGT |
| 48 | ATCCTCTATCGTTTGGCGGAGGA |
| | (SEQ ID NO: 171) |
| >strep.pneumo- | CTATCCACACCACCACGCTTATGGAAGATCGGAAGAGCGTCGTGTAGGGAAAGCTGAGCAAATGTTATCGAGGTCTA |
| 49 | GACCGCGACTGAGTTCGTTTGCA |
| | (SEQ ID NO: 172) |
| >strep.pneumo- | AAGTAACCACCTGCTGAGAGCAAGGAGATCGGAAGAGCGTCGTGTAGGGAAAGCTGAGCAAATGTTATCGAGGTCGT |
| 50 | TCTTGCGGTTCATCTGTTCCACC |
| | (SEQ ID NO: 173) |
| >strep.pneumo- | CACCTCCGCATAGCTCTTTCCTTCTAGATCGGAAGAGCGTCGTGTAGGGAAAGCTGAGCAAATGTTATCGAGGTCGG |
| 51 | AGCAGGTGCTGACACTTCTTCAT |
| | (SEQ ID NO: 174) |
| >strep.pneumo- | GATTTCACCACCAAACTTCCTCGGGAGATCGGAAGAGCGTCGTGTAGGGAAAGCTGAGCAAATGTTATCGAGGTCCG |
| 52 | TCCCTCTTAAAGAAGCAAGCCGT |
| | (SEQ ID NO: 175) |
| >strep.pneumo- | TCATTCACACCTTCATCTGGCCGAGAGATCGGAAGAGCGTCGTGTAGGGAAAGCTGAGCAAATGTTATCGAGGTCTC |
| 53 | AGCTGCATTTGGATCTGCTCCAC |
| | (SEQ ID NO: 176) |
| >strep.pneumo- | AAGGACGAGCATATCCTCTATGCCCAGATCGGAAGAGCGTCGTGTAGGGAAAGCTGAGCAAATGTTATCGAGGTCCT |
| 54 | GTATCGAGTCACATGGTCCAGCA |
| | (SEQ ID NO: 177) |
| >strep.pneumo- | CGCAAACTCGATAATGAGCTGGAGGAGATCGGAAGAGCGTCGTGTAGGGAAAGCTGAGCAAATGTTATCGAGGTCCC |
| 55 | ATTAGGATTCCAGGTCCCATTGC |
| | (SEQ ID NO: 178) |
| >strep.pneumo- | TCGGTGGTGGAGATTCAAGCTCAAGAGATCGGAAGAGCGTCGTGTAGGGAAAGCTGAGCAAATGTTATCGAGGTCGA |
| 56 | GTACACTCCAGATGTAACGGCTC |
| | (SEQ ID NO: 179) |
| >strep.pneumo- | CAATCTCTTGGTCTTGGACGAGCCAAGATCGGAAGAGCGTCGTGTAGGGAAAGCTGAGCAAATGTTATCGAGGTCAC |
| 57 | CTGCAGGTTGATGAACGAGATCG |
| | (SEQ ID NO: 180) |
| >strep.pneumo- | AGGGCACCAAGAAAGGCTTCAAAGGAGATCGGAAGAGCGTCGTGTAGGGAAAGCTGAGCAAATGTTATCGAGGTCCA |
| 58 | CGGAGACTCTTGACACTAGACTC |
| | (SEQ ID NO: 181) |

TABLE 6
Assembled pairs of homologous probe
sequences for Salmonella enterica

>sal-918813 sal-729167 163786-163885 100
GCGGTAATAGGGTGAACGTTATGGG
(SEQ ID NO: 182)
TACCCAACCGTTCACAGGTGGAAAG
(SEQ ID NO: 183)
>sal-91537 sal-495107 163787-163886 100
CGGTAATAGGGTGAACGTTATGGGC
(SEQ ID NO: 184)
ACCCAACCGTTCACAGGTGGAAAGT
(SEQ ID NO: 185)
>sal-1023952 sal-888277 163814-163913 100
GTTCCAGCGTTTGCGTTGATGCTTC
(SEQ ID NO: 186)
TGAAATTTCCGCTTCGCGGGACCAA
(SEQ ID NO: 187)
>sal-591159 sal-1123128 163815-163914 100
TTCCAGCGTTTGCGTTGATGCTTCG
(SEQ ID NO: 188)
GAAATTTCCGCTTCGCGGGACCAAA
(SEQ ID NO: 189)
>sal-244766 sal-1039899 164829-164928 100
TGATGCCGGTATTCGCTTTGGCGAT
(SEQ ID NO: 190)
ATGCGCGATTATCCCGATATTCGGC
(SEQ ID NO: 191)
>sal-379412 sal-999649 164841-164940 100
TCGCTTTGGCGATGCGGTACAACTT
(SEQ ID NO: 192)
CCCGATATTCGGCTGGATATCGATG
(SEQ ID NO: 193)
>sal-643175 sal-704852 164981-165080 100
GCCTTTGCCATCGTTTTACGCGTGA
(SEQ ID NO: 194)
GATCGTAGCATCCCCTGCATACCTT
(SEQ ID NO: 195)
>sal-231120 sal-422707 164982-165081 100
CCTTTGCCATCGTTTTACGCGTGAG
(SEQ ID NO: 196)
ATCGTAGCATCCCCTGCATACCTTG
(SEQ ID NO: 197)
>sal-1053463 sal-69659 165054-165153 100
AATTACGCCGGAAGCCGCGTTAATG
(SEQ ID NO: 198)
TGCCTTTGCCATCGTTTTACGCGTG
(SEQ ID NO: 199)
>sal-492477 sal-239882 165083-165182 100
ATGATAAAGCGTTGCGTCTCTCCGC
(SEQ ID NO: 200)
CGGGCTATATCGGTGGGAGTTTGTT
(SEQ ID NO: 201
>sal-239882 sal-596706 165157-165256 100
GAAGACTTACAGGATGGGCGGTTGT
(SEQ ID NO: 202)
ATGATAAAGCGTTGCGTCTCTCCGC
(SEQ ID NO: 203)
>sal-120080 sal-428037 2922400-2922499 100
CGAATGGCAGGACTCGCTTACTGAA
(SEQ ID NO: 204)
ACGGGCAATGCACAAATCAAAGCGG
(SEQ ID NO: 205)
>sal-662112 sal-1072150 2922404-2922503 100
TGGCAGGACTCGCTTACTGAAGATG
(SEQ ID NO: 206)
GCAATGCACAAATCAAAGCGGCGGT
(SEQ ID NO: 207)
>sal-1071952 sal-10611 2939265-2939364 100
AAACTTCGGTGCAGGGGTTAGGCAT
(SEQ ID NO: 208)
TGGCAGAGCGAGTGACTATCTGAAG
(SEQ ID NO: 209)
>sal-241367 sal-804215 4263827-4263926 100
GGCTTTGGCAACGTAGGCTTCTTCA
(SEQ ID NO: 210)
ATGCACATCACACCGTCCTGACCAA
(SEQ ID NO: 211)
>sal-8740 sal-671757 4265448-4265547 100
GCAGGCGATCTTTAATCATCTGCGG
(SEQ ID NO: 212)
AAACGCTTTCGCGTTGGCGAGGTTA
(SEQ ID NO: 213)
>sal-33849 sal-322827 4265449-4265548 100
CAGGCGATCTTTAATCATCTGCGGG
(SEQ ID NO: 214)
AACGCTTTCGCGTTGGCGAGGTTAA
(SEQ ID NO: 215)
>sal-848714 sal-549807 4265674-4265773 100
CTGCAGATCGTCCCAGTCGGATTTA
(SEQ ID NO: 216)
GTATGCAGATCGTCAGGATGGCCAA
(SEQ ID NO: 217)

(headings above the sequences in Table 6 show the identifiers of the homologous probe sequences, in respective order, followed by the genomic target coordinates, and the length of target sequence from the start of the first 25-mer to the end of the second 25-mer).

### Example 2

### Generation of M. Tuberculosis-Specific Probes

Probes specific for were made essentially as set forth in Example 1 for S. pneumoniae. Briefly, the target genome (gi 57116681 NC_{—}000962.2 Mycobacterium tuberculosis H37Rv, complete genome) was sliced into 25-mers that were filtered to have a CG content of 40% (and therefore a fixed Tₘ), and to eliminate duplicate sequences, sequences with secondary structure, and sequences with more than 4 consecutive repeats of the same nucleotide, as described in Example 1. The 25-mers were screened to also select sequences that specifically hybridize to the M. tuberculosis genomes in Table 7.

TABLE 7
M. tuberculosis additional target genomes
Target genome

gi 50953765 NC_002755.2 Mycobacterium tuberculosis CDC1551
gi 148659757 NC_009525.1 Mycobacterium tuberculosis H37Ra
gi 148821191 NC_009565.1 Mycobacterium tuberculosis F11
gi 253796915 NC_012943.1 Mycobacterium tuberculosis KZN 1435

25-mers were screened against a human genome as in Example 1 to eliminate any which would be likely specifically hybridize with human DNA. Probe sequences were screened to not specifically hybridize to the same NCBI database of microbial and viral genomes as Example 1. 25-mers were assembled in pairs into probes to capture target regions 100 nucleotides in length. The M. tuberculosis probe sequence pairs and their genomic location are listed in Table 8.

TABLE 8
Assembled pairs of homologous
probe sequences for M. tuberculosis

### mtb-gc10-5778 mtb-gc10-13476 1697202-1697301 100
>mtb-gc10-5778
ATCAGCGTCTCACGTATCTTTTGAT
(SEQ ID NO: 218)
>mtb-gc10-13476
GCTCGTTTTGATCCGATTTCTGTTT
(SEQ ID NO: 219)
### mtb-gc10-10249 mtb-gc10-21740 1697207-1697306 100
>mtb-gc10-10249
CGTCTCACGTATCTTTTGATGGAAA
(SEQ ID NO: 220)
>mtb-gc10-21740
TTTTGATCCGATTTCTGTTTCGCCA
(SEQ ID NO: 221)
### mtb-gc10-14718 mtb-gc10-21512 1697208-1697307 100
>mtb-gc10-14718
GTCTCACGTATCTTTTGATGGAAAC
(SEQ ID NO: 222)
>mtb-gc10-21512
TTTGATCCGATTTCTGTTTCGCCAA
(SEQ ID NO: 223)
### mtb-gc10-18048 mtb-gc10-20799 1697209-1697308 100
>mtb-gc10-18048
TCTCACGTATCTTTTGATGGAAACG
(SEQ ID NO: 224)
>mtb-gc10-20799
TTGATCCGATTTCTGTTTCGCCAAT
(SEQ ID NO: 225)
### mtb-gc10-13476 mtb-gc10-9738 169276-1697375 100
>mtb-gc10-13476
GCTCGTTTTGATCCGATTTCTGTTT
(SEQ ID NO: 226)
>mtb-gc10-9738
CGACGAATGCAATCAGGTCAAAATA
(SEQ ID NO: 227)
### mtb-gc10-5979 mtb-gc10-3490 1697348-1697447 100
>mtb-gc10-5979
ATCGACGAATGCAATCAGGTCAAAA
(SEQ ID NO: 228)
>mtb-gc10-3490
ACGCGGTGTCTCCAATTTAGAATAA
(SEQ ID NO: 229)
### mtb-gc10-9738 mtb-gc10-13364 1697350-1697449 100
>mtb-gc10-9738
CGACGAATGCAATCAGGTCAAAATA
(SEQ ID NO: 230)
>mtb-gc10-13364
GCGGTGTCTCCAATTTAGAATAACA
(SEQ ID NO: 231)
### mtb-gc10-1167 mtb-gc10-18133 1697421-1697520 100
>mtb-gc10-1167
AACGCGGTGTCTCCAATTTAGAATA
(SEQ ID NO: 232)
>mtb-gc10-18133
TCTGCGACATATTCAATATGGTGCT
(SEQ ID NO: 233)
### mtb-gc10-2966 mtb-gc10-6093 1697501-1697600 100
>mtb-gc10-2966
ACATATTCAATATGGTGCTCGGGAA
(SEQ ID NO: 234)
>mtb-gc10-6093
ATCGTCTCCTGTGAGATAATTGCAT
(SEQ ID NO: 235)
### mtb-gc10-10988 mtb-gc10-9385 1697583-1697682 100
>mtb-gc10-10988
CTGTGAGATAATTGCATCCGATCAT
(SEQ ID NO: 236)
>mtb-gc10-9385
CCGTTTCTGGTTTTGTCTTGATGAT
(SEQ ID NO: 237)
### mtb-gc10-15828 mtb-gc10-14219 1697591-1697690 100
>mtb-gc10-15828
TAATTGCATCCGATCATATAGGGCT
(SEQ ID NO: 238)
>mtb-gc10-14219
GGTTTTGTCTTGATGATCAAATCCG
(SEQ ID NO: 239)
### mtb-gc10-7551 mtb-gc10-12444 263241-2632440 100
>mtb-gc10-7551
CAAAACTTGATATGACCGATCTCAC
(SEQ ID NO: 240)
>mtb-gc10-12444
GATATCGCGCTATCGGTAAACTAAT
(SEQ ID NO: 241)
### mtb-gc10-8929 mtb-gc10-2100 3487428-3487527 100
>mtb-gc10-8929
CATTTACCTCTATCACTTCGGCTAA
(SEQ ID NO: 242)
>mtb-gc10-2100
AATCCGAACGAACACATAGCATTTG
(SEQ ID NO: 243)
### mtb-gc10-17338 mtb-gc10-13891 4056910-4057009 100
>mtb-gc10-17338
TCATGTTTGATAAGGCGACGAAAAC
(SEQ ID NO: 244)
>mtb-gc10-13891
GGCCTTATCTAAACCACTGAAGTTT
(SEQ ID NO: 245)
### mtb-gc10-8689 mtb-gc10-13874 4062276-4062375 100
>mtb-gc10-8689
CATCCTTATAGGAACATCACAGACT
(SEQ ID NO: 246)
>mtb-gc10-13874
GGCATTTCCGTAGCTTTTGAAATTC
(SEQ ID NO: 247)
### mtb-gc10-17547 mtb-gc10-8941 4062278-4062377 100
>mtb-gc10-17547
TCCTTATAGGAACATCACAGACTTC
(SEQ ID NO: 248)
>mtb-gc10-8941
CATTTCCGTAGCTTTTGAAATTCCC
(SEQ ID NO: 249)
### mtb-gc10-9500 mtb-gc10-7386 4062279-4062378 100
>mtb-gc10-9500
CCTTATAGGAACATCACAGACTTCA
(SEQ ID NO: 250)
>mtb-gc10-7386
ATTTCCGTAGCTTTTGAAATTCCCC
(SEQ ID NO: 251)
### mtb-gc10-11046 mtb-gc10-21368 4062280-4062379 100
>mtb-gc10-11046
CTTATAGGAACATCACAGACTTCAC
(SEQ ID NO: 252)
>mtb-gc10-21368
TTTCCGTAGCTTTTGAAATTCCCCT
(SEQ ID NO: 253)

(headings above the sequences in Table 8 show the identifiers of the homologous probe sequences, in respective order, followed by the genomic target coordinates, and the length of target sequence from the start of the first 25-mer to the end of the second 25-mer).

In addition, probe sequences were generated for specific regions of the M. tuberculosis genome, focusing on the genes where mutations have been shown to occur which confer resistance to rifampicin and isoniazid, two of the principal first-line treatments for M. tuberculosis infection.

These probes were screened for specificity as described in Example 1, but in this case were not limited to a specific Tₘ. In particular, they were designed to capture a specific 81-base region of the M. tuberculosis rpoB gene where rifampicin resistance mutations are concentrated. Two pairs of probe sequences designed to capture this region are as follows:

>mtb-H37Rv-rpoB-pr-01-H1:
(SEQ ID NO: 254)
GGTCGCCGCGATCAAGGAGTTCTTC
>mtb-H37Rv-rpoB-pr-01-H2:
(SEQ ID NO: 255)
CATCGAAACGCCGTACCGCAAGGTG
>mtb-H37Rv-rpoB-pr-02-H1:
(SEQ ID NO: 256)
GTTCATCGAAACGCCGTACCGCAAG
>mtb-H37Rv-rpoB-pr-02-H2:
(SEQ ID NO: 257)
ACCCAGGACGTGGAGGCGATCACAC

Probes specific for the M. tuberculosis inhA gene, where isoniazid resistance mutations occur, were similarly identified. A pair of probe sequences designed to capture this region are as follows:

>mtb-37rv-inha-pr-01-H1:
(SEQ D NO: 258)
TCGAACTCGACGTGCAAAACGAGGA
>mtb-37rv-inha-pr-01-H2:
(SEQ ID NO: 259)
GGCGTATTCGTATGCTTCGATGGCC

### Example 3

### Generation of Probes Directed to C. Difficile Toxin a Gene

Probes specific for the Toxin A gene of Clostridium difficile were made essentially as set forth in Example 1 for S. pneumoniae. Briefly, the target region (gi 115249003:795843-803975 Clostridium difficile 630-tcdA gene) of the target pathogen (Clostridium difficile 630) was sliced into 25-mers and filtered as set forth in example 1, to eliminate duplicate sequences, sequences with secondary structure, or sequences with more than 4 consecutive repeats of the same nucleotide. In this case, they were not screened for a fixed CG content or fixed Tₘ. Probe sequences were screened to also specifically hybridize to the following C. difficile Toxin A gene sequences:gi 260681769:718474-726606 Clostridium difficile CD196, complete genome; gi 260685375:715995-724127 Clostridium difficile R20291, tcdA gene; and gi 144925 gb M30307.1 CLOTOXACD C.difficile toxin A gene, complete cds. The 25-mers were screened against a human genome as in Example 1 to eliminate any which would be likely to cross-hybridize with human DNA. The probe sequences were screened to not specifically hybridize to the same NCBI database of microbial and viral genomes as Example 1. Probe sequence pairs were assembled to capture target regions of 100 to 200 nucleotides in length. The pairs for Clostridium difficile Toxin A probes are listed below in Table 11, which includes the genomic location information for each pair of probe sequences:

TABLE 9
Assembled probe sequences for C. difficile

>cdif-toxA-1.L50 pos1467-1566
CTCGCTCCACAATAAGTTTAAGTGG
(SEQ ID NO: 260)
ATTCAGCTACCGCAGAAAACTCTAT
(SEQ ID NO: 261)
>cdif-toxA-1.L120 pos1467-1566
CTCGCTCCACAATAAGTTTAAGTGG
(SEQ ID NO: 262)
ATTCAGCTACCGCAGAAAACTCTAT
(SEQ ID NO: 263)
>cdif-toxA-2.L50 pos8185-8284
TGATGGAGTAAAAGCCCCTGGGATA
(SEQ ID NO: 264)
CTTTATGCCTGATACTGCTATGGCT
(SEQ ID NO: 265)
>cdif-toxA-2.L120 pos8185-8284
TGATGGAGTAAAAGCCCCTGGGATA
(SEQ ID NO: 266)
CTTTATGCCTGATACTGCTATGGCT
(SEQ ID NO: 267)
>cd if-toxA-3.L100 pos3114-3263
ATAACAGAGGGGATACCTATTGTAT
(SEQ ID NO: 268)
CCTCAGTTAAGGTTCAACTTTATGC
(SEQ ID NO: 269)
>cdif-toxA-3.L170 pos3114-3263
ATAACAGAGGGGATACCTATTGTAT
(SEQ ID NO: 270)
CCTCAGTTAAGGTTCAACTTTATGC
(SEQ ID NO: 271)
>cdif-toxA-4.L150 pos1528-1727
ATAAATAGTCTATGGAGCTTTGATC
(SEQ ID NO: 272)
TTTTATGCCAGAAGCTCGCTCCACA
(SEQ ID NO: 273)
>cd if-toxA-4.L250 pos1528-1727
ATAAATAGTCTATGGAGCTTTGATC
(SEQ ID NO: 274)
TTTTATGCCAGAAGCTCGCTCCACA
(SEQ ID NO: 275)

### Example 4

### Generation of Probes for Detection of Drug-Resistance Mutations in HIV

This example provides a method of selecting probes that will detect the presence of HIV-1 and that will detect drug resistance mutations. A list of 65 drug resistance loci in the HIV RT, protease, fusion, and integrase genes was first generated. These loci were taken from the HIV Drug Restistance Database at Stanford University and the tables at the following websites:

http://hivdb.stanford.edu/cgi-bin/NRTIResiNote.cgi
http://hivdb.stanford.edu/cgi-bin/NNRTIResiNote.cgi
http://hivdb.stanford.edu/cgi-bin/PIResiNote.cgi
http://hivdb.stanford.edu/cgi-bin/FIResiNote.cgi
http://hivdb.stanford.edu/cgi-bin/INIResiNote.cgi

A set of 1522 HIV genomic sequences was also downloaded from NCBI. Using the BioPerl module Bio::Tools::dpAlign, the position of each resistance mutation in each of the 1522 genomic sequences was determined. For each genome, each gene was aligned against all three frames and both orientations to determine the best alignment. The resistance mutation positions were then mapped from the consensus sequence to the genomic sequence.

As input to the probe design pipeline, 100 of the 1522 HIV genome sequences were chosen at random. To generate the set of candidate probe sequences (probe arms), the list of all n-mers which have a length of from 20 to 30 and which occurred within 50 bases of any resistance mutation in any of the 100 input sequences was generated. These n-mers were chosen as they were the candidate probe sequences that would generate a sequencing read that will reveal at least one of the resistance mutations. Duplicates were removed from the list of n-mers, as were n-mers containing homopolymer runs having a length of greater than three and certain other underdesirable sequences (e.g., restriction sites associated with enzymes that might be used during microarray synthesis of probes). The candidate probe sequences were further filtered to retain only those present in 20 or more of the 100 input HIV strains.

The probe design software then generated two scores for each n-mer describing its desirability as a ligation-side probe arm and as an extension-side probe arm. The scores were generated as described herein, and the distribution of desirable probe arm melting temperatures was selected to be two degrees higher than usual. Once each candidate probe arm had been scored, the best candidate is selected from the set sharing a common prefix of length 20, where the best candidate was identified by the highest sum of the score as a ligation-side probe arm and the score as an initiation-side probe arm. Candidate probe arms that scored poorly (i.e., those that had an expected probability of working of less than 0.25) were discarded from further consideration. This process accomplished the goal of examining candidate probe arms with varying lengths (from 20 to 30 nucleotides) to find the one with the best melting temperature and other characteristics.

Each remaining probe arm was then aligned against two exclusion databases—human genome sequences (February 2009 human reference sequence [GRCh37/hg19] produced by the Genome Reference Consortium; available at http://genome.ucsc.edu/cgi-bin/hgGateway) and sequences present in U.S. Pat. No. 6,252,059-using the short read aligning program Bowtie (available at http://bowtie-bio.sourceforge.net/index.shtml). Any candidate probe arm that matched either database with one or zero mismatches was discarded. Remaining candidate probe arms were then aligned with the 100 HIV target genomes using Bowtie.

The target list of resistance mutation sites to be covered by probe capture regions was then prepared. The list contains one entry for every known resistance mutation as mapped to each strain (i.e., 65*100=6500 entries). The probe arm selection process was then designed to choose probe arms such that the sequencing reads of at least two probe arms include each entry on the list (i.e., each mutation site in each strain).

For each candidate probe arm, the number of resistance mutation sites in the list of 6500 that would be covered by the probe arm's sequence read if the probe arm is used as a ligation-side probe arm and as an initiation-side probe arm was determined. This was done by examining the Bowtie alignment of the candidate probe arm against each genome and counting the number of restistance mutation sites within a fixed distance (50 bases) of the probe arm's location. This step takes into account the number of HIV strains to which the candidate probe arm is a good match.

The 100 HIV target strains were processed in an arbitrary order to generate candidate completed probes (i.e., pairs of probe arm sequences for assembly into a completed probe) for each strain based on candidate probe arm sequences that occur within 85 to 250 bases of each other in that strain. Each candidate probe was retained only if the expected probability that the probe works is greater than 0.5. Then, the list of resistance mutations (out of the 6500) that will be covered by sequencing reads from this probe was completed; this represents the coverage list. This computation combines the lists from the two candidate probe arms that were joined to form the probe, retaining entries for a genome only if the candidate probe arms were within 300 bases and in the correct orientation in that genome.

The candidate probes were sorted based on the sum of the coverage list for each probe and the probe with the highest sum, i.e., the probe that covers the greatest number of resistance mutations, was chosen.

The coverage lists for the remaining candidate probes was updated to reflect resistance mutations that have already been covered by two probes. Probes were removed from consideration that do not cover any uncovered resistance mutations.

In the practice of this probe selection process, if no probes remain or if all resistance mutations have been covered by two probes, the process may cease. If probes remain, the candidate list may again be sorted based on the sum of the coverage list for each probe and the probe with the highest sum, i.e., the probe from the list that covers the greatest number of resitance mutations may be chosen.

In some cases, mutations were introduced into the probe arms of all selected probes. The mutations were generated by trying variations on each position in the probe arm, starting from the backbone side and working towards the capture side, until the probe arm had no match of more than 19 base pairs with any of the 1522 HIV genomes. The melting temperatures of all such variations on the probe arm were computed and the variation that caused a decrease in melting temperature (based on the imperfect duplex of the original and mutated probe arms as computed by Melting 5.0.3 (available at http://www.ebi.ac.uk/compneur-srv/melting/melting5-doc/melting.html) closest to 1.5 degrees was retained as the new probe arm. Thus, by increasing the desired melting temperature in the initial parameters and attempting to achieve a lower melting temperature with the mismatch, the final probe arms may behave similarly to unmutated probes under experimental conditions.

The mutated probe arms were then aligned with Bowtie against all 1522 HIV genomes to determine how many of the 1522 would be captured by at least one probe and how many of the 65 resistance mutations across the 1522 strains were captured (though there are 1522*65, or 98930, total loci in theory, 86,905 loci were identifiable, as not all resistance mutations could be mapped to all strains). Based on this analysis, the set of target strains was augmented, and the process was repeated on 323 strains. The original 100 strains, plus 223 new strains that were captured by few or no probes in the initial round, were used. The only change to the initial parameters was that the candidate probe arms that are found in seven or more strains, rather than the original 20, were retained.

The final step of the probe design process was to filter the 467 preliminary probe sequences to remove probes that might cross-hybridize or cross-prime with other probes in the pool. This filtering was based on alignments of the probes to each other and to themselves, followed by melting temperature computations on the aligned regions to determine the likelihood of the duplex forming under experimental conditions. This filtering removed 34 probes as likely to form hairpins and 56 probes as likely to cross-prime with other probes, leaving 376 probes. These 376 probes contain at least one probe for 1384 of the 1522 strains. Some probes capture over two hundred strains while many capture just one or several; this generally reflects the order in which the probes were selected, as probes that captured resistance mutations in many strains were chosen first, and probes specific to one or several strains were chosen last.

### Example 5

### Generation of Probes Differentiating Strains of HPV

This example provides a method selecting probes that will detect and distinguish publicly available genomes of 288 sequenced strains of human papilloma virus (consisting of 137 distinct types, wherein some types have multiple isolates or strains). The goal of the probe selection process was to pick probes such that the sequence reads from the region of interest captured by these probes would reveal at least seven SNPs or small indels between any pair of strains.

The probe design pipeline began by generating a list of all n-mers of length 18 to 26 from all 288 strains. N-mers were then discarded which contained a homopolymer stretch having a of length of greater than three or which contained certain restriction enzyme sites (certain enzymes are used to process probes that have been synthesized on a microarray, so such sites may not be allowed in probe sequences in some embodiments to ensure that all probes are compatible with all possible synthesis options). Each of the remaining 9,825,946 n-mers was then scored, as described for the HIV-specific n-mers in Example 4, according to its desirability as a ligation-side probe arm and as an initiation-side probe arm. As in Example 4, the highest-scoring probe with a given 18-base prefix was retained. The methods further filtered the probes to remove those with a perfect or 1-base pair mismatch to the human genome, leaving 715,533 for use in probe selection.

A square matrix was constructed with each of the 288 HPV strains along each axis (though only the upper half of the matrix is used to indicate each pairwise result only once in the square matrix). Each entry in the matrix indicated the number of SNPs or small indels that the methods attempts to cover with the expected reads from the probes it selects. Thus, this matrix is the matrix of desired SNPs, i.e., the matrix showd how many differences the finished probe set is selected to reveal between any pair of strains. In this case, all entries were set (or “initialized”) to seven. Other probe design tasks might initialize the matrix differently. For example, if two strains were considered clinically identical, the matrix might have a zero entry for those strains, indicating that there is no need to distinguish them. If certain strains need higher coverage, entries corresponding to those strains may contain higher values.

To determine the utility of each n-mer as a probe arm, the probe selection methods were used to determine how many SNPs between pairs of strains are revealed by the n-mer. Thus, the n-mers were aligned against the set of 288 strains using Bowtie, and allows one mismatch in alignment of each n-mer. For each n-mer and each pair of strains to which the n-mer aligns (in an order-independent fashion), an alignment of the two regions downstream of the n-mer was performed to determine the number of SNPs and small indels that would be observed from a sequencing read through each region if this n-mer were used as the ligation-side probe arm. The length of the flanking region used in the alignment depends on the expected sequencing read length; in this case, a flanking region of 50 bases was used. An alignment of the 50 bases upstream of the n-mer was also performed to determine the number of SNPs and small indels that would be detected if the n-mer were used as an initiation-side probe arm. Thus, for each n-mer, two matrices of observed differences between pairs of strains were computed: one matrix for the n-mer as a ligation-side probe arm and the other as an initiation-side probe arm. An example of the alignment for one n-mer is shown below, where an asterisk indicates 100% identity at that position, and where the strain is indicated at left:

| (SEQ ID NO: 276) | |
|---|---|
| FM955841 | AGTTGTTGCAACAGCATTGCGACTATATCTGGGTTA |
| (SEQ ID NO: 277) | |
| M32305 | AGCTGTTGCAACAGCATTGTGACTATATATGGGTCC |
| (SEQ ID NO: 278) | |
| FM955838 | AGTTATTGCAACAGCATTGTGACTATATTTGGATTA |
| (SEQ ID NO: 279) | |
| D90252 | AGCTGTTGCAACAGCATTGTGACTATATCTGGGTCC |
| (SEQ ID NO: 280) | |
| M22961 | AGCTATTGCAACAGCATTGTGACTATATCTGGGTCC |
| (SEQ ID NO: 281) | |
| NC_001531 | AGCTATTGCAACAGCATTGTGACTATATCTGGGTCC |
| | ** * *********************** *** * |

This n-mer reveals three SNPs between strains FM955841 and M32305, none between M22961 and NC_{—}001531, and six between FM955838 and D90252.

To construct probes containing a pair of n-mers, all 288 HPV strains were processed in an arbitrary order and probes were generated for each strain by combining n-mers that fell within 300 bases of each other. Each candidate probe was scored based on the following values (1) and (2):
(1) The probability that the probe will work, and
(2) the expected number of SNPs or small indels that the probe will reveal between strains. The expected number of SNPs or small indels that the probe will reveal between strains was obtained by summing the observed SNP/indel matrices for the two probe arms. Values corresponding to strains in which the probe will not work (e.g., the probe arms are too far apart or in the wrong strand orientation) were set to zero. Furthermore, the maximum value in the matrix was set to the lesser of 3 or the value of the corresponding entry in the target matrix. The final number for the probe was the sum over all entries in this matrix.
   The final score for a probe was the product of values (1) and (2).

The probe with the highest score was then selected and then subtracted the probe's observed SNP/indel matrix value from the desired target matrix (negative values in the result were set to zero). The score for the remaining probes was then updated; scores may only decrease during this process as the remaining probes may detect differences between strains that have already been covered by a selected probe. Probe selection continued in this manner, i.e., selecting probes and rescoring the remaining candidate probes, until the target matrix contained all zeros (meaning that the selected probes will reveal at least seven SNPs or indels between each pair of strains) or until no remaining candidate probe has a non-zero score (meaning that no remaining candidate probe will reveal differences between strains that have not already been detected).

This iterative probe selection process selected 548 probes. Filtering the probes for hairpins, cross-priming, and cross-hybridization as in Example 4 left 346 probes.

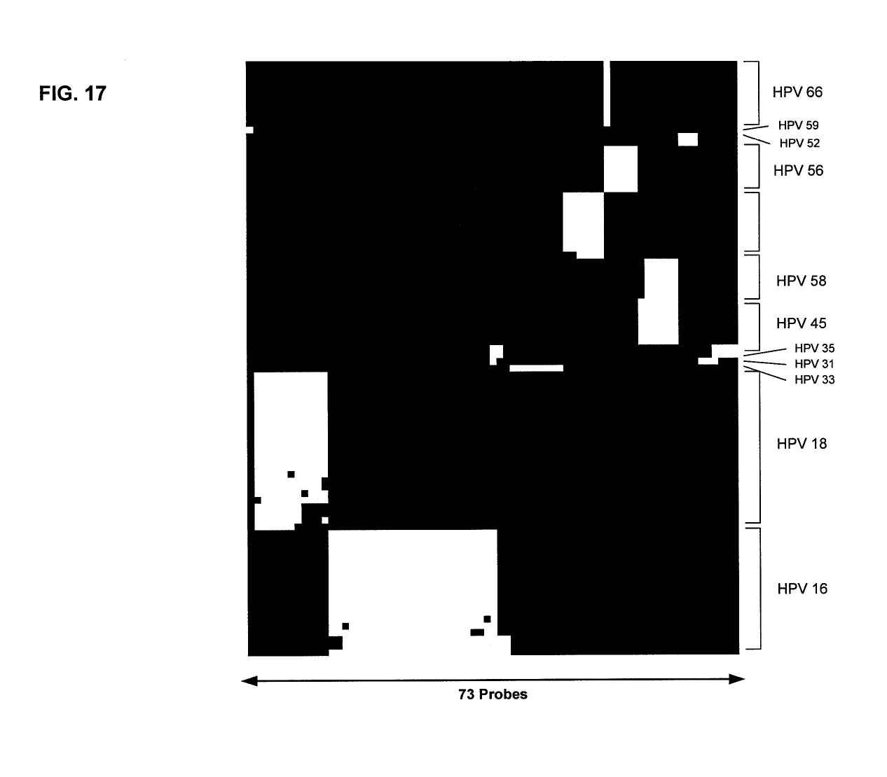

When a simulation of HPV strain detection is performed using these 346 probes and a set of high-risk HPV strains (HPV 16, 18, 31, 33, 35, 39, 45, 51, 52, 56, 58, 59), 73 probes were expected to produce a product. FIG. 17 shows the matrix of which probes (x-axis) worked against which strains (y-axis) in the simulation, with a white block indicating an expected product and a black block indicating that the probe did not produce a product from that strain.

### Example 6

### Detection of HPV Strains in Clinical Samples

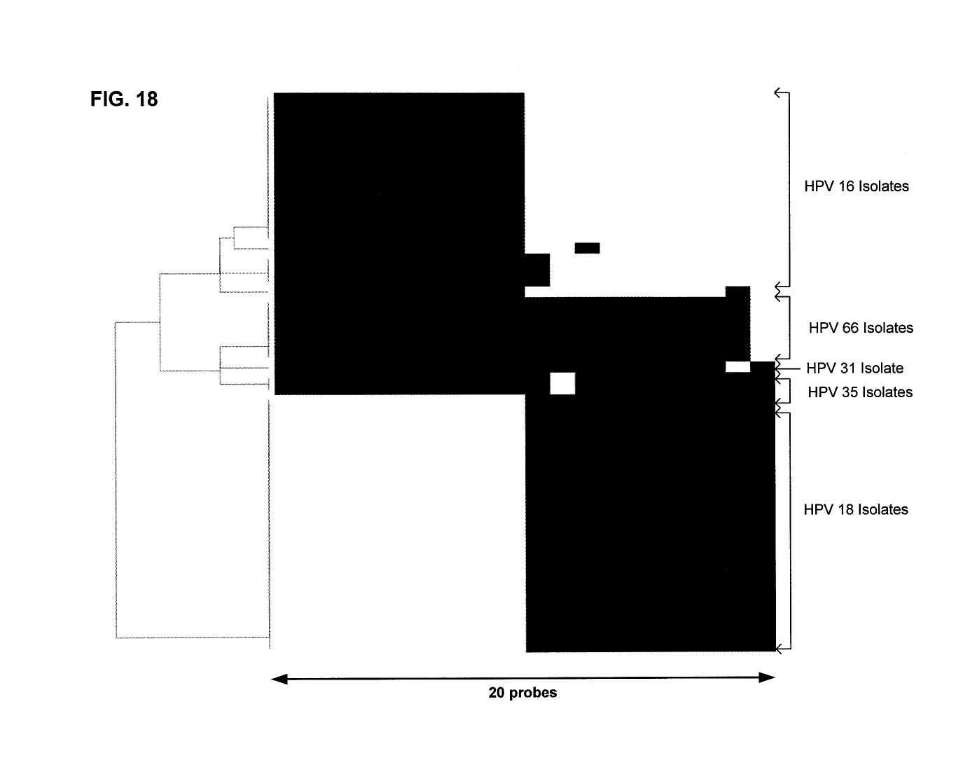

FIG. 18 depicts a target matrix for a group of 20 specific HPV probes versus target HPV strain genomes. Probes are represented across the x-axis of the plot, and strains are represented along the y-axis. White areas indicate probes predicted to bind to the genome of the corresponding strains indicated, while black areas indicate probes that are not predicted to bind to the corresponding strains.

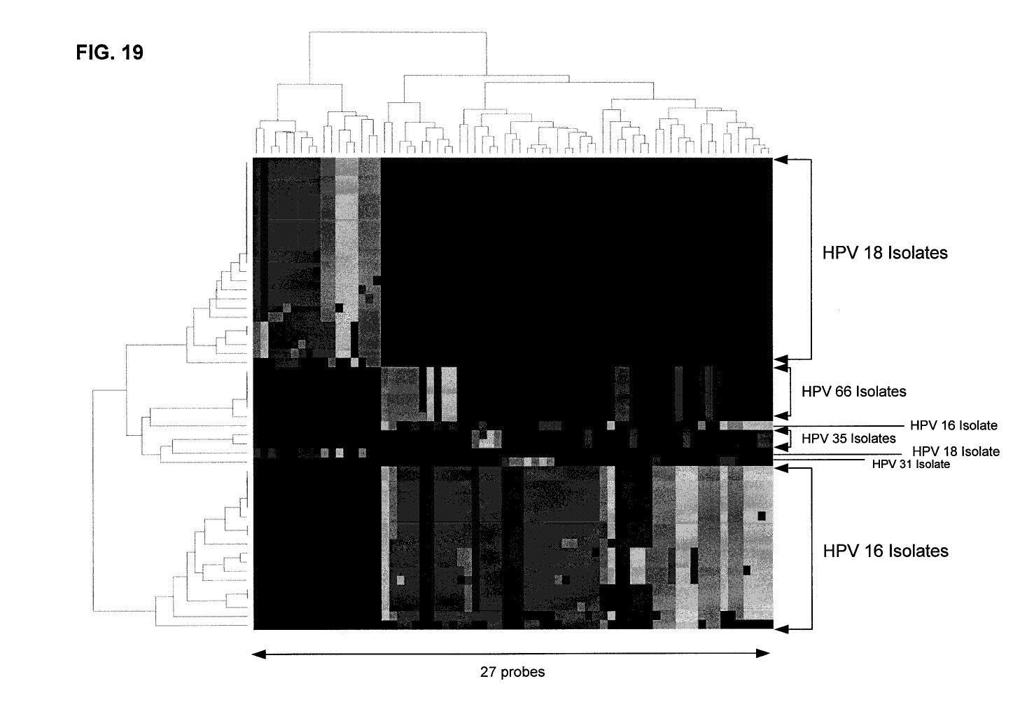

FIG. 19 depicts a target matrix expanded to indicate the number and type of SNPs identified by each of 27 specific HPV probes. Different grayscale shading indicates any particular base changes to each of T, C, G, or A, or the presence of an indel Gray=Indel, and black indicated no read from that strain at that location. Individual probes are indicated along the x-axis, and each probe is broken up into one column, or multiple columns if it captures more than one SNP.

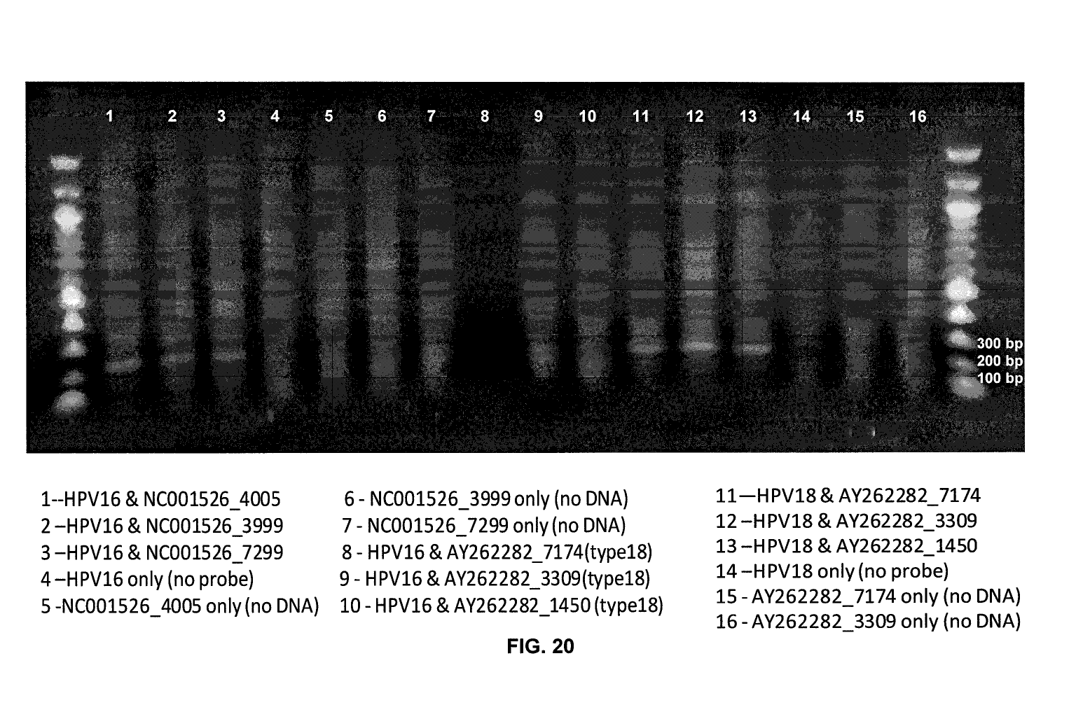

Using methods as described herein, HPV 16-directed probes (NC001526_{—}4005, NC001526_{—}3999, or NC001526_{—}7299) or HPV 18-directed probes (AY262282_{—}7174, AY262282_{—}3309, or AY262282_{—}1450) were combined with DNA from clinical samples (ThinPrep) containing either HPV 16 and 18, as indicated by the lane number for specific samples in the gel shown in FIG. 20. After hybridization and subsequent gap-filling polymerase extension and ligation (circularizing capture), PCR was performed to detect circularized probes. PCR amplicons were detected at the expected size (250 nt) in several samples (indicated by lanes 1-3 and 11-13). The HPV 16-directed probes detected HPV 16, and the HPV 18-directed probes detected HPV 18 but not HPV 16.

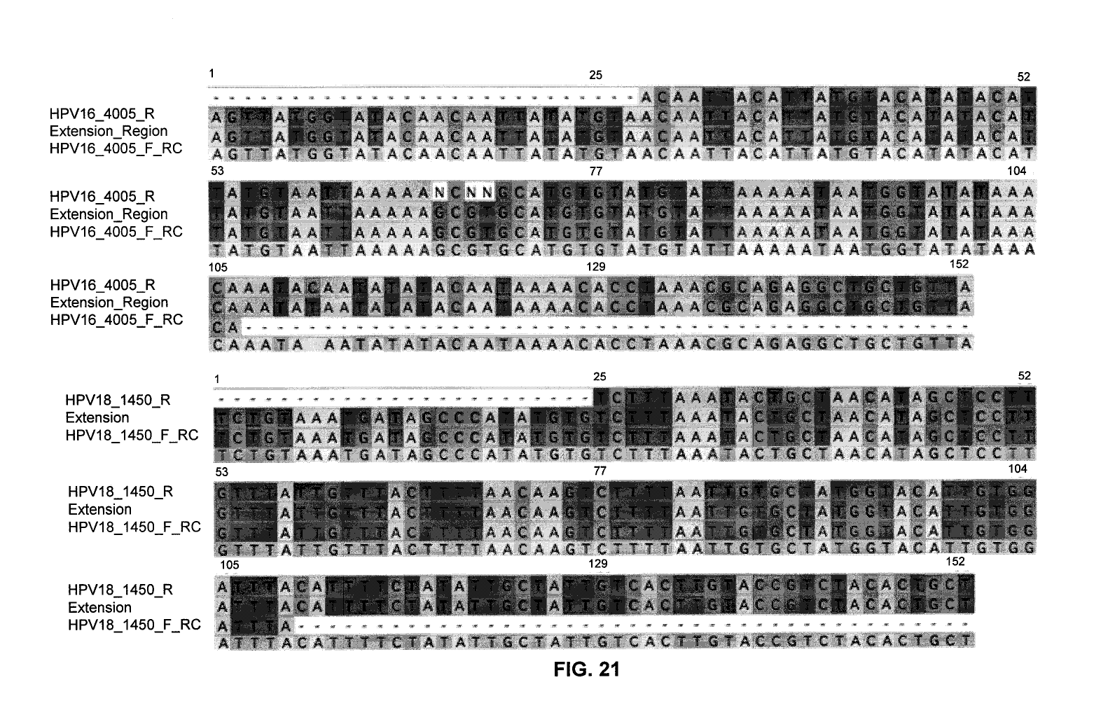

FIG. 21 shows an example alignment of Sanger sequencing of amplicons generated in the samples corresponding to FIG. 20 above. Sequences aligned to HPV 16 and HPV18 reference genomes, and indicated sequence capture through the polymerase extension region.

### Example 7

### Detection of Bacterial DNA in Clinical Samples

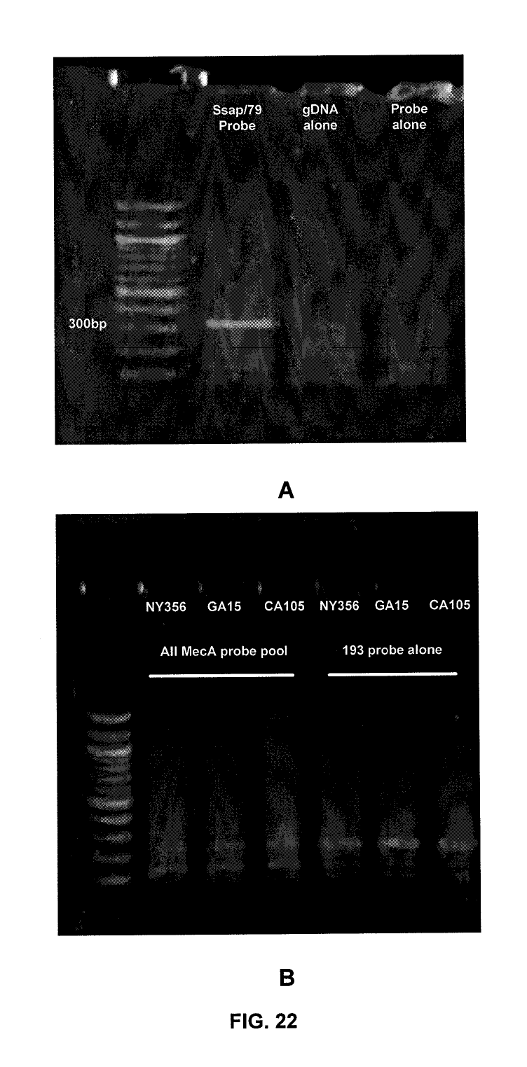

Staphylococcus saprophyticus genomic DNA was detected in clinical samples from patients with urinary tract infection (UTI) using a single S. saprophyticus-directed probe in a circularizing capture as described herein (FIG. 22A). S. saprophyticus DNA was also detected in bacterial clinical isolates using either a single probe (“193” probe) or a pooled mixture of probes comprising probes directed to the MecA gene region (“All MecA probe pool”) (FIG. 22B) (bands of the expected size are visible in all samples; clinical isolates are denoted as NY356, GA15, and CA105).

Sanger sequencing in forward and reverse directions indicated polymerase extension and capture of target gDNA using the Staphylococcus saprophyticus-directed probe of FIG. 22A, as observed in an alignment of observed sequencing reads of the PCR-amplified circularized probe with genomic DNA from a reference Staphylococcus saprophyticus strain.

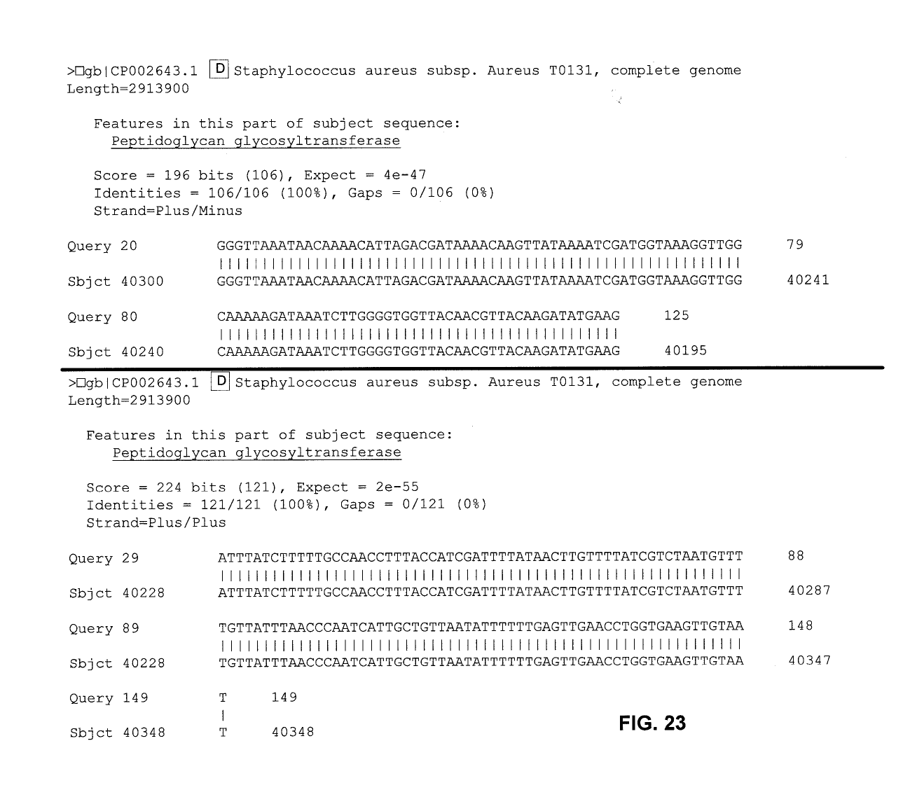

Sanger sequencing also indicated polymerase extension and capture of Staphylococcus aureus target gDNA when combined with Staphylococcus aureus-directed probes, as shown in the alignment of observed sequencing reads of the PCR-amplified circularized probe with genomic Staphylococcus aureus sequences (FIG. 23).

### Example 8

### Detection of Viral DNA in Clinical Samples

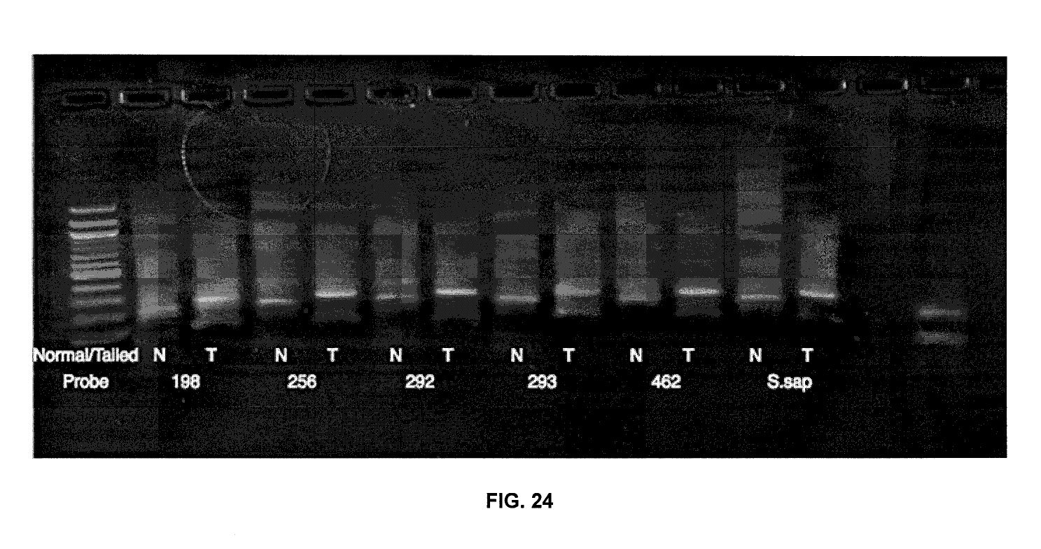

cDNA reverse transcribed from RNA isolated from cultured influenza virus was also detected using five individual molecular inversion probes and amplification for normal Sanger (N) or Next generation sequencing (T, tailed primer) is shown in FIG. 24 (probes denoted as 198, 256, 292, 293, and 462; S.sap denotes Staphylococcus saprophyticus genomic DNA control).

### Example 9

### Multiplex Detection of Bacterial DNA in Clinical UTI Samples

A pool of 60 completed probes directed to organisms with potential roles in urinary tract infections was prepared at a concentration of 3 nM total nucleic acid, containing equal molar proportions of each probe.

The probe pool was hybridized to approximately 4 μl of 33 individual clinical urinary tract infection (UTI) samples and four control samples for 24 hours. Each clinical sample was quantified by picogreen to contain variable amounts of dsDNA between 0.1 pg and 100 ng per microliter.

Polymerase gap filling, ligation, and digestion reactions were performed, and any circularized product was amplified by universal primers containing a 3′ portion that hybridizes to the universal backbone of the probe, and a 5′ tail containing adaptor sequences required for hybridization to an IIlumina flow cell (Illumunia Inc., San Diego, Calif.). Individual 3′ primers containing non-hybridizing six-nucleotide barcode inserts were used to label amplicons from each individual clinical sample with a unique DNA sequence tag to allow subsequent identification of sequence reads from this sample.

Amplicons of the expected size were excised after being resolved on a 2% agarose gel. Amplicons were purified from excess agarose and salts in preparation for sequencing. All samples were multiplexed together into a single sequencing run on an IIlumina GAII instrument by barcoding each of the 37 samples with a six-nucleotide barcode. These samples were further multiplexed with additional samples (and different barcodes) that were not included in this analysis. The sequencing run produced roughly thirty-three million reads.

The probe arms for the 60 UTI probes were aligned to a large collection of genomes and partial genomes. For each match to each probe, an “expected read” was assembled that consisted of the left probe arm, the extension region, the right probe arm, and the 21-nucleotides of backbone sequence between the six-nucleotide barcode and the right probe arm. A Bowtie database was built of these 10,886 expected reads.

To align the reads, the FASTQ file produced by the Illumina base-calling software was first split into separate files, one for each barcode. Each barcode (the first six nucleotides of the read) was compared to all known barcodes. A read was assigned to a barcode if the barcode portion of the read had a single match to a barcode that was better than the match to any other barcode. The quality of the match to a barcode is the sum of base qualities at positions where the sequencing read and expected barcode mismatch; thus, a high quality match has a low sum (ideally zero) and the matching from reads to barcodes accounts for the quality of the sequencing read.

Each of the 37 barcodes used in the experiment yielded at least one read, with a range from 11,245 to 4,874,885 reads per barcode. The reads for each barcode were aligned separately against the probe database using Bowtie version 0.12.7 with command line options “-p 8-q—trim5 6-solexa1.3-quals-e 200-best—strata-m 20-k 20”. Thus, the Bowtie aligner only returned hits of the sequencing reads against the expected reads that were of the best match quality (i.e., if several expected reads matched the sequencing read with the same number of mismatches, both reads were included in the output. However, another expected read that has one more mismatch would not have been included, as its match would not have been as good as those of the best quality. See Bowtie's documentation of “—best—strata” for more details). Each bowtie alignment was fed into an analysis script. For each read, the script determined the set of strains from which the read plausibly came (that is, the set of strains corresponding to the expected reads that the read matched at the best quality). This set of strains could be written as a set of Genbank accession numbers, e.g., “ACLE01000080, GG668578, NC_{—}010554” or could be written as the set of strains corresponding to these accession numbers. For example, “ACLE01000080, GG668578, NC_{—}010554” were three Proteus mirabilis strains. A different read may map equally well to expected reads from “ABVP01000025, ACLE01000080, GG661996, GG668578, NC_{—}010554” which includes both Proteus mirabilis and Proteus penneri. For example, the analysis script might report::

236—Proteus mirabilis (ACLE01000080, GG668578, NC_{—}010554)
1—Proteus penneri, Proteus mirabilis (ABVP01000025, ACLE01000080, GG661996, GG668578, NC_{—}010554),
   indicating that 236 reads map to expected products from P. mirabilis and one read maps to expected products from P. mirabilis or P. penneri. Thus, these results were interpreted to indicate the presence of P. mirabilis, as it is more likely that the single read from the second line was actually from P. mirabilis rather than being a co-infection by P. penneri.

The results from the 37 different samples indicates infections by a variety of different organisms. For example, the analyis script reported the following for sample #7:
2—Aggregatibacter aphrophilus, Proteus penneri, Proteus mirabilis (ABVP01000025, ACLE01000080, GG661996, GG668578, NC_{—}010554, NC_{—}012913)
324—Candida albicans (AJ251858)
6—Klebsiella pneumoniae (ACZD01000012, EU682505, GG703525, NC_{—}009648, NC_{—}011283, NC_{—}012731)
30109—Klebsiella pneumoniae (ACZD01000012, EU682505, GG703525, NC_{—}009648, NC_{—}012731
5—Klebsiella pneumoniae (ACZD01000013, EU682505, GG703525, NC_{—}009648, NC_{—}012731)
7—Klebsiella pneumoniae, Escherichia coli (ACZD01000012, EU682505, GG703525, NC_{—}009648, NC_{—}010378, NC_{—}012731, NC_{—}013503)
2—Klebsiella pneumoniae, Escherichia coli, Klebsiella variicola (ACZD01000012, EU682505, GG703525, NC_{—}009648, NC_{—}010378, NC_{—}011283, NC_{—}012731, NC_{—}013503, NC_{—}013850)
30—Klebsiella pneumoniae, Escherichia coli, Klebsiella variicola, Citrobacter koseri (ACZD01000012, EU682505, GG703525, NC_{—}009648, NC_{—}009792, NC_{—}010378, NC_{—}011283, NC_{—}012731, NC_{—}013503, NC_{—}013850)
4—Klebsiella pneumoniae, Klebsiella variicola (ACZD01000012, EU682505, GG703525, NC_{—}009648, NC_{—}011283, NC_{—}012731, NC_{—}013850)
656—Klebsiella pneumoniae, Klebsiella variicola (ACZD01000013, EU682505, GG703525, NC_{—}009648, NC_{—}011283, NC_{—}012731, NC_{—}013850)
2—Lactobacillus helveticus, Lactobacillus delbrueckii (ACLM01000017, AEAT01000083, CP000156, CP002429, GG700753, NC_{—}008054, NC_{—}008529, NC_{—}010080, NC_{—}014727)
549—Proteus mirabilis (ACLE01000080, GG668578, NC_{—}010554)
27—Proteus penneri, Proteus mirabilis (ABVP01000025, ACLE01000080, GG661996, GG668578, NC_{—}010554)
7—Providencia rettgeri, Providencia alcalifaciens, Proteus penneri, Proteus mirabilis, Providencia rustigianii (ABVP01000025, ABXV02000043, ABXW01000004, ACCI02000067, ACLE01000080, GG661996, GG668578, GG703820, GG705265, NC_{—}010554)
76—Staphylococcus saprophyticus (AF144088, AP008934, NC_{—}007350)
310—Ureaplasma parvum (CP000942, NC_{—}002162, NC_{—}010503)
25—Ureaplasma urealyticum (CP001184, NC_{—}011374)
5—Ureaplasma urealyticum, Ureaplasma parvum (CP000942, CP001184, NC_{—}002162, NC_{—}010503, NC_{—}011374)

The vast majority of the reads in this analysis report came from Klebsiella pneumoniae, a know common cause of urinary tract infections. The data also indicate the low-level presence of other known urinary tract infectants, including Candida albicans and Ureaplasma parvum.

The results for the sample of Candida albicans genomic DNA showed 293,384 reads from C. albicans as well as a few hundred reads from Klebsiella and Proteus, presumably either due to low contamination of the cell culture used to produce the DNA (less than 0.1%, based on the read counts) or sequencing errors that caused reads from other samples to appear to contain the barcode for this sample.

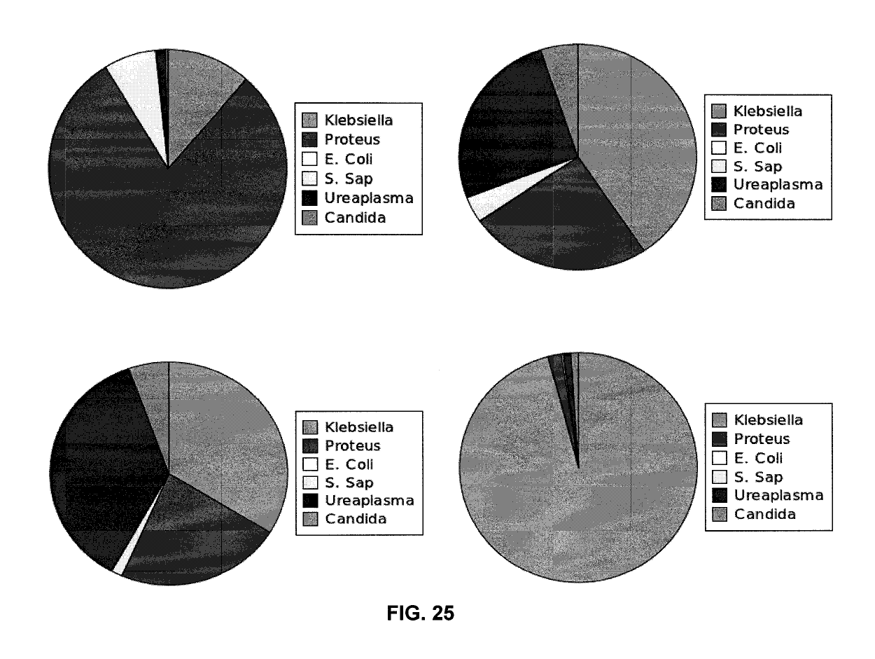

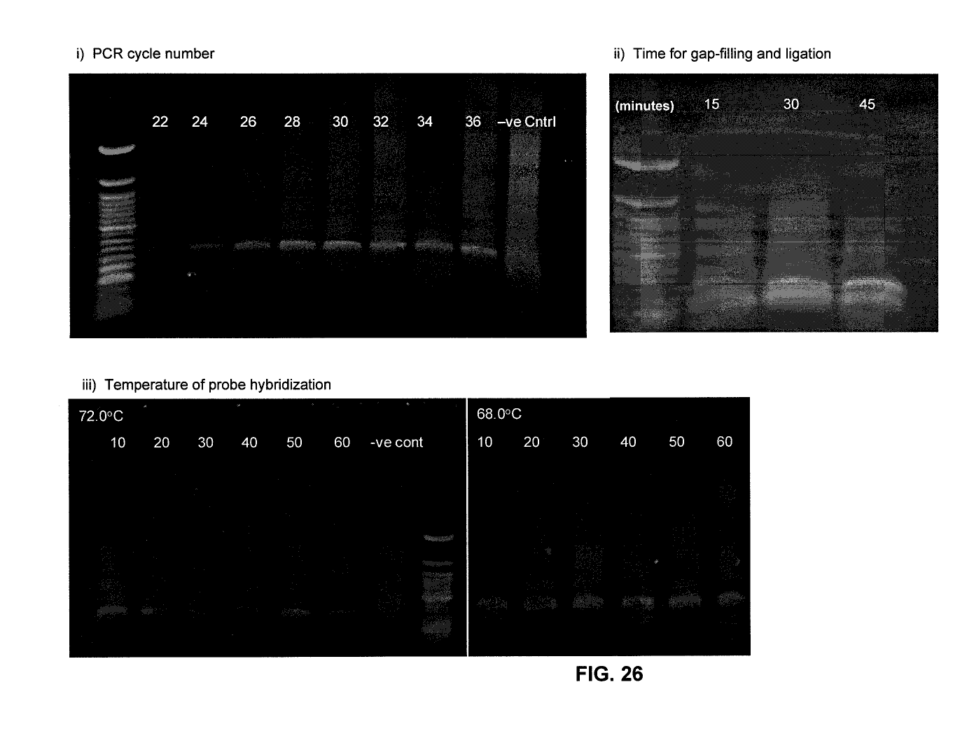

The proportions of different infectious species in detected in four of the urinary tract infection samples from this sequencing run are shown in FIG. 25. The different primary infections were identified as Proteus, Klebsiella, and Ureaplasma infections.

### Example 10

### Circularizing Capture Reaction Methods

The circularizing capture protocol may be performed using a varying number of PCR cycles to determine an optimum number of PCR cycles (FIG. 25(i)) for particular probes and target DNA samples.

The protocol may also be performed using varying lengths of time for gap filling and ligation. In some cases, gap filling is complete after only 15 minutes of incubation (FIG. 25(ii)).

Probe hybridization may be performed at slightly varying temperatures to determine the optimum hybridization temperature for specific probes. At either 72° C. or 68° C., for example, substantial circularized product is generated after hybridization for time periods as short as 10 minutes (FIG. 25(iii)); incubation time in minutes is indicated for each lane).

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patent applications, and patents cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. The recitation of series of numbers with differing amounts of significant digits in the specification is not to be construed as implying that numbers with fewer significant digits given have the same precision as numbers with more significant digits given.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

Unless otherwise indicated, the term “at least” preceding a series of elements is to be understood to refer to every element in the series. Those skilled in the art will recognize, or be able to ascertain using no more than routine experimentation, many equivalents to the specific embodiments described herein. Such equivalents are intended to be encompassed by the following claims.

Unless defined otherwise, all technical and scientific terms used herein have the same meaning as commonly understood by one of ordinary skill in the art to which the invention belongs. Any methods and materials similar or equivalent to those described herein can be used in the practice or testing of the invention.

The publications discussed herein are provided solely for their disclosure prior to the filing date of the present application. Nothing herein is to be construed as an admission that the invention is not entitled to antedate such publication by virtue of prior invention. Further, the dates of publication provided may be different from the actual publication dates which may need to be independently confirmed.

Other embodiments of the invention will be apparent to those skilled in the art from consideration of the specification and practice of the embodiments disclosed herein. It is intended that the specification and examples be considered as exemplary only, with a true scope and spirit of the invention being indicated by the following claims.

What is claimed is:

1. A mixture comprising a plurality of probes and/or oligonucleotide primer pairs for detecting at least one target organism in a subject, wherein each probe or oligonucleotide primer pair comprises:
a. a first homologous probe sequence that specifically hybridizes to a first target sequence present in the genome of the at least one target organism; and
b. a second homologous probe sequence that specifically hybridizes to a second target sequence present in the genome of the at least one target organism; and
c. wherein each probe further comprises, a backbone sequence in between the first and second homologous probe sequences comprising a detectable moiety and a primer,
wherein the first target sequence and the second target sequence are separated by a region of interest comprising at least two nucleotides, and wherein each of the first and second homologous probe sequences in each probe:
i. specifically hybridizes to the target organism;
ii. has a Tₘ in the range of 50-72° C.;
iii. does not specifically hybridize to (a) any other homologous probe sequence in the mixture; (b) any backbone sequence (c) any nucleotide sequences present in the genome of the subject; or (d) any nucleotide sequences present in the genome of a predetermined set of sequenced organisms other than the target organism;
iv. occurs in the at least one target genome below a repeat threshold, wherein the repeat threshold is 20; and
v. does not contain more than 4 consecutive identical nucleotides and is substantially free of secondary structure.

2. The mixture of claim 1, wherein each of the first and second homologous probe sequences specifically hybridize to the genome of sequenced variants of the organism of interest adjacent to the region of interest and the region of interest is polymorphic amongst sequenced variants of the organism of interest, and optionally wherein the region of interest is associated with toxin production or antibiotic resistance.

3-15. (canceled)

16. The mixture of claim 1, wherein the mixture comprises at least one probe and/or oligonucleotide primer pair for at least 4, 10, 15, 20, 30, 40, 60, 80, 100, 150, 200, 250, 300, 400, 500, 1000, 2000, 4000, 8000, 10000, 15000, or 20000 different target organisms.

17. The mixture of claim 1, wherein the mixture comprises at least 10, 20, 30, 40, 60, 80, 100, 200, 250, 500, 1000, 2000, 4000, 8000, 10000, 20000, 30000, 40000, 50000, 60000, 70000, 80000, 90000, or 100000 probes and/or oligonucleotide primer pairs.

18. The mixture of claim 1, wherein the mixture further comprises at least one subject-specific probe and/or oligonucleotide primer pair, wherein the subject is a human.

19-30. (canceled)

31. The mixture of claim 1, wherein the mixture further comprises extracted nucleic acids from a biological sample, wherein said sample is from a human patient.

32-33. (canceled)

34. The mixture of claim 1, further comprising at least one sample internal calibration standard nucleic acid at least one probe and/or oligonucleotide primer pair that specifically hybridizes with the sample internal calibration standard nucleic acid.

35-36. (canceled)

37. The mixture of claim 1, wherein the mixture comprises at least one homologous probe sequence, or the reverse complement thereof, from any one of Tables 4, 5, 6, 8, or 9.

38. The mixture of claim 1, wherein the region of interest is at least 2, 4, 8, 10, 20, 40, 60, 80, 100, 125, 150, 200, 250, 300, 350, 400, 500, 600, 700, 800, 900, 1000, 1200, 1400, 1600, 1800, or 2000 nucleotides.

39-40. (canceled)

41. A method of detecting the presence of one or more target organisms comprising:
a) contacting a test sample suspected of containing a target organism with the mixture of claim 1;
b) capturing a region of interest by at least one probe and/or oligonucleotide primer pair hybridized to a first and second target sequence; and
c) detecting the captured region of interest, thereby detecting the presence of the one or more target organisms.

42-51. (canceled)

52. The method of claim 41, further comprising the step of sequencing the region of interest, and analyzing the sequence of the captured region of interest with respect to the sequence of known genomes and a model of sequencing errors to estimate the proportions or abundances of the various organisms present in the sample.

53-54. (canceled)

55. The method of claim 41, wherein the test sample is obtained from a human subject.

56-57. (canceled)

58. The method of claim 41, further comprising the steps of adding a sample internal calibration standard nucleic acid to the test sample and detecting the sample internal calibration standard nucleic acid.

59. (canceled)

60. The method of claim 41, further comprising providing a therapeutic recommendation based on the at least one target organism detected.

61-63. (canceled)

64. A method of treating a subject infected with a pathogen, comprising the method of claim 41 and further comprising the steps of detecting the presence of at least one pathogen and administering a suitable prophylaxis to the subject based on the at least one pathogen detected.

65. A method of making the mixture of claim 1, comprising:
a) providing at least one reference genome for an organism of interest, at least one non-hybridizing genome, and optionally at least one hybridizing genome that is not identical to the reference genome;
b) slicing the reference genome into n-mers, wherein n is in the range of 18-50;
c) identifying a set of screened n-mers from the sliced reference genome, wherein the set of screened n-mers:
i) is non-repetitive;
ii) consists of n-mers that are substantially free of secondary structure;
iii) is free of n-mers containing more than 4 consecutive identical nucleotides;
iv) consists of n-mers with a Tm in the range of 50-72° C.; and
d) identifying a set of homologous probe sequences, wherein the homologous probe sequences consist of screened n-mers, wherein:
i) the n-mers do not specifically hybridize to any non-hybridizing genome;
ii) the n-mers occur 1-20 times in the reference genome and optional at least one hybridizing genome; and
e) assembling a plurality of probes and/or oligonucleotide primer pairs, wherein each probe or oligonucleotide primer pair comprises a first homologous probe sequence and a second homologous probe sequence, wherein:
i) the first and second homologous probe sequences specifically hybridize to a first and second target sequence in the genome of the organism of interest, respectively, and wherein the first and second target sequences are separated by a region of interest comprising at least two nucleotides;
ii) the plurality of probes do not specifically hybridize to each other; and
iii) the plurality of probes are substantially free of secondary structure.

66. The method of claim 65, wherein two or more reference genomes are provided, and wherein, at least one probe and/or oligonucleotide primer pair hybridizes to at least one of the reference genomes.

67. (canceled)

68. The method of claim 65, wherein the probes and/or oligonucleotide primer pairs in the mixture are scored and selected based upon a threshold number of polymorphisms that are present between known sequences within a set of genomic sequences of a region of interest.

69-70. (canceled)

71. The method of claim 65, wherein each probe or oligonucleotide primer pair is altered such that no homologous probe sequence contains a perfect match of more than a specified length to a set of exclusion genomes, and wherein the altered sequence will still hybridize to one or more target genomes.

72. (canceled)

73. The method of claim 65, further comprising repeating steps (a)-(e) for each number m of additional organisms of interest, wherein m is greater than 4, 10, 15, 20, 30, 40, 60, 80, 100, 150, 200, 250, 300, 400, 500, 1000, 2000, 4000, 8000, 10000, 15000, or 20000.

74-75. (canceled)

76. The method of claim 65, wherein the at least one non-hybridizing genomes comprises a predetermined set of sequenced organisms other than the target organism, optionally wherein the at least one non-hybridizing genome comprises the human genome.

77. (canceled)

78. The method of claim 65, wherein the slicing of the genome into n-mers is with an offset between 1 and n.

79-81. (canceled)

82. The method of claim 65, wherein the method takes under 16, 14, 12, 10, 8, 6, or 4 days; or 72, 48, 36, 24, 12, 10, 8, 6, or 4 hours using a single core Pentium Xeon 2.5 ghz processor on a target genome of at least 10, 9, 8, 7, 6, 5, 4, 3, or 2 megabases.

83-84. (canceled)
